SEQUENCE LISTING (30 sequences)

SEQ ID NO: 1 - protein, 912 aa, Homo sapiens
MAGPSNPPATMKIAVIGQSLFGQEVYCHLRKEGHEVVGVFTVPDKDGKADPLGLEAEKDGVPVFKYSRWRAKGQALPDVVAKYQALGAELNVLPFCSQFIPMEIISAPRHGSIIYHPSLLPRHRGASAINWTLIHGDKKGGFSIFWADDGLDTGDLLLQKECEVLPDDTVSTLYNRFLFPEGIKGMVQAVRLIAEGKAPRLPQPEEGATYEGIQKKETAKINWDQPAEAIHNWIRGNDKVPGAWTEACEQKLTFFNSTLNTSGLVPEGDALPIPGAHRPGVVTKAGLILFGNDDKMLLVKNIQLEDGKMILASNFFKGAASSVLELTEAELVTAEAVRSVWQRILPKVLEVEDSTDFFKSGAASVDVVRLVEEVKELCDGLELENEDVYMASTFGDFIQLLVRKLRGDDEEGECSIDYVEMAVNKRTVRMPHQLFIGGEFVDAEGAKTSETINPTDGSVICQVSLAQVTDVDKAVAAAKDAFENGRWGKISARDRGRLMYRLADLMEQHQEELATIEALDAGAVYTLALKTHVGMSIQTFRYFAGWCDKIQGSTIPINQARPNRNLTLTRKEPVGVCGIIIPWNYPLMMLSWKTAACLAAGNTVVIKPAQVTPLTALKFAELTLKAGIPKGVVNVLPGSGSLVGQRLSDHPDVRKIGFTGSTEVGKHIMKSCAISNVKKVSLELGGKSPLIIFADCDLNKAVQMGMSSVFFNKGENCIAAGRLFVEDSIHDEFVRRVVEEVRKMKVGNPLDRDTDHGPQNHHAHLVKLMEYCQHGVKEGATLVCGGNQVPRPGFFFEPTVFTDVEDHMFIAKEESFGPVMIISRFADGDLDAVLSRANATEFGLASGVFTRDINKALYVSDKLQAGTVFVNTYNKTDVAAPFGGFKQSGFGKDLGEAALNEYLRVKTVTFEY

SEQ ID NO: 2 - DNA, 3204 bp, Homo sapiens
tctgcggcaccaggactgagtagaagggagagagtggagaaggggaattgcagagagaaaaccaggggctgtttttctctcggagaggcgggtaggcactgggcgggcagaagcgccgctatccacccggatgcgcagctgctaaggggccgcctctgcaagcggctgcaaattcccggagggcagcgtctcctttcgctctgctgtgtccgtagcacatggcaggtccttccaaccctcctgctaccatgaagattgcagtgattggacagagcctgtttggccaggaagtttactgccacctgaggaaggagggccacgaagtggtgggtgtgttcactgttccagacaaggatggaaaggccgaccccctgggtctggaagctgagaaggatggagtgccggtattcaagtactcccggtggcgtgcaaaaggacaggctttgcctgatgtggtggcaaaataccaggctttgggggccgagctcaacgtcctgcccttctgcagccaattcatccccatggagataatcagtgccccccggcatggctccatcatctatcacccgtcactgctccctaggcaccgaggggcctcggccatcaactggaccctcattcacggagataagaaaggggggttttccatcttctgggcggatgatggtctggacaccggagacctgctgctgcagaaggagtgtgaggtgctcccggacgacaccgtgagcacgctgtacaaccgcttcctcttccctgaaggcatcaaagggatggtgcaggccgtgaggctgatcgctgagggcaaagcccccagactccctcagcctgaggaaggagccacctatgaggggattcagaagaaggagacagccaagatcaactgggaccagccggcagaggccattcacaactggatccgcgggaacgacaaggtgccgggagcctggacagaggcctgtgaacagaaactgacatttttcaactcaacgctgaacacttcaggcctggtgcccgagggagacgctttgcccatcccaggagcccatcggccaggggtggtcaccaaagcaggactcatcctctttgggaatgatgacaaaatgctgctggtgaagaatattcagctggaggatggcaaaatgatcctggcctcgaacttctttaagggggcagccagcagtgtccttgagctgacagaggcagagctggttactgcggaggctgtgcggagtgtttggcagcggatcctccccaaagtcctggaggttgaagactccactgatttcttcaagtcaggggccgcgtctgtggacgttgtgaggctggtggaggaagtgaaggagctgtgtgatggcctggagttagaaaatgaagatgtgtacatggcatccacctttggggacttcatccagctgttagtgaggaagctgcgaggggacgatgaggagggcgagtgcagcattgactacgtggaaatggcagtgaacaagcgcactgtccgcatgccccaccagctcttcattgggggggagttcgtggatgccgagggcgccaagacctctgagaccatcaatcccaccgatggaagtgtcatctgccaggtatccctggcccaagtcaccgacgtcgacaaggcagtggccgcagccaaggatgcctttgagaatggacggtgggggaagatcagtgcgcgggaccggggccggctgatgtacaggttggcagatctcatggagcagcaccaggaggagctggccaccattgaggccctggatgcgggtgccgtctacacgctggccctgaagacccacgtgggcatgtccatccagaccttccgctactttgctggctggtgtgacaagatccagggctccaccatccccatcaaccaggccagacccaaccgcaacctgaccttgaccaggaaggagcctgttggggtttgtggcatcatcatcccctggaactatcccctgatgatgctgtcctggaagacagctgcctgcctggctgccgggaacacagtggtgatcaagcctgctcaggtgaccccactcacagccttgaagtttgcagagctgacattaaaggccggcattcccaaaggtgtggttaacgtcctcccaggatctggctccctggtcggccagagactctcagaccatcctgatgtgaggaaaatcgggttcacaggctccacagaggtgggcaagcacatcatgaaaagctgtgccataagtaacgtgaagaaggtgtccctggaactgggcgggaagtcacccctcatcatctttgctgactgtgacctcaacaaggctgtgcagatggggatgagttctgttttcttcaacaaaggagagaattgcattgcagcaggccgactctttgtggaggactccattcatgatgagttcgtgcggagagtggtagaagaggtgcggaagatgaaggtgggcaacccgctggacagggacaccgaccacgggccgcagaatcaccatgcccaccttgtgaagctgatggagtactgccagcatggcgtgaaggaaggggccacactggtctgcggcgggaatcaggtccctcggccagggttcttctttgagccaactgttttcacagacgtggaagaccacatgttcatagccaaggaggagtccttcgggcctgtcatgatcatctctcggtttgctgatggggacttggatgccgtgctgtctcgggccaatgccacggaatttggcctggcttctggtgtcttcaccagggacatcaacaaggccctgtatgtcagtgacaagctccaggcaggcactgtgtttgtcaacacgtacaacaagaccgacgtggccgctcccttcggaggattcaaacagtctggatttggcaaagatctaggagaggcggctctgaacgagtacctgcgggtcaagacagtgaccttcgaatactgaagaaaggtctttgtgagaagaaagtccctgcccctccctcgtggctggggccccctccctcttgagcctgggtgcacagcacctcccacctggggggctagtggaagccctcctgcctgcacaccatgtctgcatcttggacgccctctgtccagtcagaagcagcccttggctgggtgaggtgtgcccctcccagggagaataaagcttctgaagagagaccgtccacaaaaaaaaaaaaaaaaaa

SEQ ID NO: 3 - protein, 923 aa, Homo sapiens
MLRRGSQALRRFSTGRVYFKNKLKLALIGQSLFGQEVYSHLRKEGHRVVGVFTVPDKDGKADPLALAAEKDGTPVFKLPKWRVKGKTIKEVAEAYRSVGAELNVLPFCTQFIPMDIIDSPKHGSIIYHPSILPRHRGASAINWTLIMGDKKAGFSVFWADDGLDTGPILLQRSCDVEPNDTVDALYNRFLFPEGIKAMVEAVQLIADGKAPRIPQPEEGATYEGIQKKENAEISWDQSAEVLHNWIRGHDKVPGAWTEINGQMVTFYGSTLLNSSVPPGEPLEIKGAKKPGLVTKNGLVLFGNDGKALTVRNLQFEDGKMIPASQYFSTGETSVVELTAEEVKVAETIKVIWAGILSNVPIIEDSTDFFKSGASSMDVARLVEEIRQKCGGLQLQNEDVYMATKFEGFIQKVVRKLRGEDQEVELVVDYISKEVNEIMVKMPYQCFINGQFTDADDGKTYDTINPTDGSTICKVSYASLADVDKAVAAAKDAFENGEWGRMNARERGRLMYRLADLLEENQEELATIEALDSGAVYTLALKTHIGMSVQTFRYFAGWCDKIQGSTIPINQARPNRNLTFTKKEPLGVCAIIIPWNYPLMMLAWKSAACLAAGNTLVLKPAQVTPLTALKFAELSVKAGFPKGVINIIPGSGGIAGQRLSEHPDIRKLGFTGSTPIGKQIMKSCAVSNLKKVSLELGGKSPLIIFNDCELDKAVRMGMGAVFFNKGENCIAAGRLFVEESIHDEFVTRVVEEIKKMKIGDPLDRSTDHGPQNHKAHLEKLLQYCETGVKEGATLVYGGRQVQRPGFFMEPTVFTDVEDYMYLAKEESFGPIMVISKFQNGDIDGVLQRANSTEYGLASGVFTRDINKAMYVSEKLEAGTVFINTYNKTDVAAPFGGVKQSGFGKDLGEEALNEYLKTKTVTLEY

SEQ ID NO: 4 - DNA, 7555 bp, Homo sapiens
gcggcgagccgcgagccaggcagtccggggcatccagactgcaggccgcgcccaggccgcgcccaggctgcgccgcccgcctgcctcccgcgctgccgcgtcgccagtgctagcgctcctctccagcatgctgcggcggggcagccaggcgctccggcgcttctccactggccgggtttatttcaaaaacaagctgaagttggcactaattggccagagcctctttggacaagaagtctatagccacctccgcaaagagggccaccgagtagtaggggtgttcacagttccagacaaggatggaaaagctgaccctctggctttggctgcagagaaagatgggacccctgtgttcaagcttcctaaatggagggtcaagggcaagaccatcaaagaagtggcagaagcctacagatccgtgggtgcagagctaaatgtgctccctttctgcactcagttcattcccatggatataattgatagtccaaagcacggctctatcatttatcacccatccatcctgcccaggcacagaggagcctctgctatcaattggactctaattatgggagataagaaagctgggttttctgttttctgggctgatgatggcttggatacaggacccatccttcttcagagatcatgtgatgttgaacccaatgatacagtggatgcactttataatcggtttctttttcctgaaggaatcaaggccatggtagaagctgtccaactcatagctgatggaaaagctcctcgtataccccagccagaagaaggggcaacatatgaaggtatccagaaaaaggaaaatgctgagatttcttgggaccagtctgccgaagttttacataactggattcgaggtcatgataaagtccctggagcttggacagagataaatggacagatggtcactttctatggctcgacattactgaatagctctgtgcctcctggagaaccactggaaattaaaggtgccaagaagcctggtctcgttaccaaaaatggacttgttctttttggtaacgatggaaaagcactgacggtgagaaatctgcagtttgaagatggaaaaatgatccctgcctctcagtacttttcaacgggtgagacgtcagtggtagaactgacagctgaagaggtgaaagtggcagagaccatcaaggtcatctgggctggaattttaagcaatgtccccattattgaagactcaacagacttctttaaatctggagcaagctcaatggatgttgccaggctggttgaagagatcagacagaaatgtggtgggcttcagttgcagaatgaagatgtctatatggccaccaagtttgaaggctttatccaaaaggtcgtgaggaaactgagaggagaagatcaagaggtggagctggttgtagattatatttcaaaggaggtcaatgaaatcatggtaaaaatgccataccagtgtttcataaatggacagttcacagatgcagacgatggaaagacttacgacactatcaacccaacagatggatctacaatatgcaaagtatcctacgcttctttggcggatgttgataaagcagtagcagcagcaaaagatgcttttgaaaacggtgaatggggaagaatgaatgcaagagaaagaggaagattgatgtatagacttgcagacctactggaagagaaccaagaagagctggcaactattgaagcccttgattcaggggctgtctataccttggccctgaagacacacattggaatgtctgtgcaaacattcagatattttgctggctggtgcgacaaaattcagggttctactattccaatcaaccaggcccgtccaaatcgcaatctgaccttcaccaagaaagagccactcggtgtctgtgccattattattccctggaactacccgctgatgatgctggcatggaagagtgctgcgtgtttggcagcaggcaataccttagtgctcaagccagcacaggtcacgcccttgactgctttgaagtttgcagaactgtctgtgaaagcaggctttccaaagggggtcatcaacatcattccaggctcaggtggcatagcaggacaacgtctgtctgaacatcctgacatccgcaaacttggtttcactggatccactcctattggcaaacagatcatgaagagctgtgctgttagcaacttgaagaaagtttcccttgagcttggtggcaagtctccacttataatatttaatgactgtgaacttgacaaggctgtgcgaatgggcatgggagcagtatttttcaacaaaggagagaactgtattgctgctgggcggttgttcgtggaagaatccatccacgacgaatttgtgacaagagtggtagaagaaattaaaaagatgaaaattggtgatccacttgacagatccactgatcatgggccccaaaatcataaggctcatctggaaaagctgctgcaatactgtgaaactggagtgaaagaaggggccactttggtgtacgggggaagacaagtccaaaggccaggctttttcatggagccgaccgtgttcacagatgtggaagactacatgtacctcgccaaagaggaatcctttgggcctattatggtcatttctaaattccaaaatggggacatcgatggagtgttgcagcgagcaaatagtacagagtatggtttggcctcaggggtttttacaagagacataaacaaagctatgtatgtgagtgaaaaactggaagcaggaactgtttttattaacacatacaacaagacagatgtggcggccccatttggcggagttaaacaatctggctttggaaaagacttaggtgaggaagctctaaatgaatatctcaaaaccaagacggtgacactggaatattagagcaacaccatcatcaggaaagccttgacagacagccctttacaactctggacacacttaagaagattgggtgtgttgaggcaggaggtgtcagccacaaaccaaaaaatacacagatggaccatgaagagggccaggccatgttaaagcatttacacatgtgcctgagtattttctaatacaccttccagtgatttggagttgttgcattttgactatgttgtatatcatacgtatttctaaaataccaagctgtttctcccctacctagacaaatctattcatggttcccatcttgaagatgtcagtaccatgcagttataatacacaaggtgcatttattggaaactttgtataatatgtacaggtttttaacctctgaactatacatagggggttattaaaaagattttctataagtcttctaaggaacagtataacctgtaaggaatgtgaaggtagttcttttttagtatttggaaataagatacatctttgtgcctttgatattccattttttaacccactgtgatgggtgatcaacctagaaacattatcttgagtacctactaggtaccaggtactatattatgttctgaggagtatagagaatttaatgatatgatggctggcccccacatagtttaaattttagtaaatagcttttgaagcaaattttacatatgatatagtagaaggctgatcctggtcgtatcataccatcttcctatctatgtaactttgggaaactctcgcaactcctctgagcctctgcttccctatgtgtaaaacagggatagtaaatgccttcctcaggacccttaataggagaattcattgcagtaatgtaagtaaagcacctcacattaatgctttgctcatggtaagtactcaaatttaactctgatttcctccgtcaccattcttaaaagatattgagatagtttaattaactagatgaattcatttcccacaacccttttcaatcatcaattcctagatatttttctcatccattgttctgacacaatgcctgatacagcagcactgaaaaatgccacacaatgaaaaatggcaatagtacaaggaaaaggggtgcttttctttgggcagctcgctcgtccttcatgggacatcttactttccatttttctacctattggttctgctgttcactggctgtgtgatcttgggcaagatagtaatctaatatctcagagcctaggttgagtatctataaaatgaaaatcaaatctctatctcagtaggtgttgcaaggattcagtgagataatatacataatgcacttaacaaggcgtttggaccatagcattgaagaaatggaaactattaacagcccatttcccattggcagacagaagtagtcaggtgagtaaattttcaccatctatgtgtgactagaaggcggcaaatttctgaatcacatgagtctccaaaagatagccagaaagttaaattctattaatcctcctttaaaaataaaatttcagtaaacattcctttttctttggctttgaagaagccttagggaatatttgtcattttggagacttggcagaataacatgaggggattgtagggaatcaataaaaactaaacaacaaaatcagagtcagagaacattttcaaaaggaagaataggaggtttgatcccagcatgataaacagagcgaatttggcctggaagcacttttgattatactatagctcatttaccatcccagagtttggcacagctgaaattttaagttggaatgaatattcactgggcccaaaatgacagttcatatttgaataaaagtgacaaaagcctttttataagtaatcacttttaagtgaaatgttttaactgatttcatgtgatttagaatatgatttaatcaaattattttaatgatagatggaatggcagacaaaaacatgcctgtccttctagactgattttactttaccctctaatattcatctcagtagcagtgttttaaatattctctgggctgcaaaactctttgggaatctgataaaagctatgaacactccctgtgtcccgcttctacccccaaaattcatgtgcacacacacaattctgcaagtatcttcaaagggttcacagacctcccaaaggccatgcttgggccccagattaagaactcctttctccatagcaagttttaaacatttcttaccagcttacatttttagatctggctgatcagaatcaaaggctctgtgtaatacataaagttaccaagtgaactggaattggaacatcaccctccccagcctgctaggtgatttacttaacacatagagtaataaaatcatcgctgttgctttagatcacggattattttgctaataatgctaaggatgaagctgtgatcttattatcacctgaatcgggaggtgtggacactttaagcagttccactttccttctaattccccatccccatgcctttgctaaagctgtcccttttgctctaacacccttcctggaccttcctaccctagctgggctaagtgtttctcctcagcgttcccacttgtttcaaacatagcacttaccacttgtactaaaattacttgccttcttaattagatatgaacaaccctccccaactccagtatgggccttctgtcaataataatacgatatgacagctaccatttattaagggcctcctgtatgaaagaccttaggctaagcatgttttaaatgttatttaatcttcacaatctctgaaaaaaatgaagaaatcaacgtgcttttcttactacctctacccctaagccattattacttttttttttttttgagacagagttttgctcttgttgcccaggctgcagtgcagtggtgcaatcttggctcactgcaacctctgcctcttgggttcaagcgattgtcatgccttagccttccaagtagctgggattacaggtgtgtgccactacacctggctaagtagagatggggtttcgccatgttggccaggctggtcttgaactcctgacctcaagtgatccacctgcctccgcctcccaaagtgctgggattacaggcatgaaccactgcacctggcctgttacctctttcctacaattttgctcaagtctcccaactggtcttctggattcctctcttctgcggtcctgttcaaagcttaagtcagacagtgtcacttcactcgtctgtttaaaacctttcaatggcccccatttcacgtagaccaaagtccaacgtatttacctggcctactgatcttgctcctagctacctctgacctcatctcctgtcaatttccctctcattctgttccaccatcctgactgccttgacttcctcaacagaacaagcctgctcctgcctcagggcctctgtccttattcttcctcttcccaggggtgtgctggtaaaatatttaacaaatagttctccgggacgggggagaaaaccctcatttgtagcatttgcaggtatctatgtgtaaatactctcatcaaggctatttttgagccactaatttgccttcactgaatacagagtttgggaagagatgcatgccatcagaacaaatgcaagccagcaccagcacaccactgcctcttcctgcaactcttgtccatacacaacctcatggctggctggctcacttcctgcaggtctctcctcaaatatcatctgatgagagacacattccctgactatgctttctaaaataggccatatgcccccacattcataccccatctgctgtcattctttattctttttataagtgcattattttcatagcacttatcactacctgttgtatattaatcaatgatcttttcccattagaatgtaagtttcatgaacaggtacttgttttaatactgtatctccagtcctaatgtgtaacaggagcccaataaatgtttgctttcaaatggagaggttaagtaacctgctcaaatcacacagctattaagtggcagaacaggttttcaagcaatgcatctggtggttttaactaagtcgagatagtttttattcctaatgcctaaatcagggcctaggtagtgagctgtgggcacatattaagtattggttaaactaaaaataataagcaaaatggacattatctataaaagcttttgtggaaatggctagagctagggtaaggaaacaaatttggttccccatacctgccctccaagaaaataaagctgtcaaggaaaatctgggctaagagtaggatatgagggatgatggataaggcatgagacatgagaaataagggggattaaattattattactattatacaaatgatgcctgagtagatttttaaaatgattaaatacccaatgatgtaaaaaacatttataaaataggaaagtaagactgactcaaccataatttgttgagtcaacccaaaaatctatttggttattttcaaacagaaatagcctacagatgatatctgagattgttccaaactttttctatgaatatgtatactttttttacataattaacataatactgtatattaatttgttacctgctttttcaattaacaatatatcataagcatctatgccaataaacacaattctgcatatttcaaaaaaaaaaaaaaaaaa

SEQ ID NO: 5 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 6 - DNA, 1213 bp, Homo sapiens
tggaggcctggctggtgctcacatacaataattaactgctgagtggccttcgcccaatcccaggctccactcctgggctccattcccactccctgcctgtctcctaggccactaaaccacagctgtcccctggaataaggcaagggggagtgtagagcagagcagaagcctgagccagacggagagccacctcctctcccaggaactgaacccaaaggatcacctggtattccctgagagtacagatttctccggcgtggccctcaagggacagacatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctgacctccttttaccttctgatacctggaaatccctgccctgttcagccccacagctcccaactatttggttcctgctccatggtcgggcctctgacagccactttgaataaaccagacaccgcacatgtgtcttgagaattatttggaaaaaaaaaaaaaaaaaa

SEQ ID NO: 7 - protein, 587 aa, Homo sapiens
MSRARSHLRAALFLAAASARGITTQVAARRGLSAWPVPQEPSMEYQDAVRMLNTLQTNAGYLEQVKRQRGDPQTQLEAMELYLARSGLQVEDLDRLNIIHVTGTKGKGSTCAFTECILRSYGLKTGFFSSPHLVQVRERIRINGQPISPELFTKYFWRLYHRLEETKDGSCVSMPPYFRFLTLMAFHVFLQEKVDLAVVEVGIGGAYDCTNIIRKPVVCGVSSLGIDHTSLLGDTVEKIAWQKGGIFKQGVPAFTVLQPEGPLAVLRDRAQQISCPLYLCPMLEALEEGGPPLTLGLEGEHQRSNAALALQLAHCWLQRQDRHGAGEPKASRPGLLWQLPLAPVFQPTSHMRLGLRNTEWPGRTQVLRRGPLTWYLDGAHTASSAQACVRWFRQALQGRERPSGGPEVRVLLFNATGDRDPAALLKLLQPCQFDYAVFCPNLTEVSSTGNADQQNFTVTLDQVLLRCLEHQQHWNHLDEEQASPDLWSAPSPEPGGSASLLLAPHPPHTCSASSLVFSCISHALQWISQGRDPIFQPPSPPKGLLTHPVAHSGASILREAAAIHVLVTGSLHLVGGVLKLLEPALSQ

SEQ ID NO: 8 - DNA, 2487 bp, Homo sapiens
gcggggcgtctcccgcccgggcctagagcgctgccgggggcgccgggactatgtcgcgggcgcggagccacctgcgcgccgctctattcctggcagcggcgtctgcgcgcggcataacgacccaggtcgcggcgcggcggggcttgagcgcgtggccggtgccgcaggagccgagcatggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgctggaggccctcgaggaaggggggccgccgctgaccctgggcctggagggggagcaccagcggtccaacgccgccttggccttgcagctggcccactgctggctgcagcggcaggaccgccatggtgctggggagccaaaggcatccaggccagggctcctgtggcagctgcccctggcacctgtgttccagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccgggacccggcggccctgctgaagctgctgcagccctgccagtttgactatgccgtcttctgccctaacctgacagaggtgtcatccacaggcaacgcagaccaacagaacttcacagtgacactggaccaggtcctgctccgctgcctggaacaccagcagcactggaaccacctggacgaagagcaggccagcccggacctctggagtgcccccagcccagagcccggtgggtccgcatccctgcttctggcgccccacccaccccacacctgcagtgccagctccctcgtcttcagctgcatttcacatgccttgcaatggatcagccaaggccgagaccccatcttccagccacctagtcccccaaagggcctcctcacccaccctgtggctcacagtggggccagcatactccgtgaggctgctgccatccatgtgctagtcactggcagcctgcacctggtgggtggtgtcctgaagctgctggagcccgcactgtcccagtagccaaggcccggggttggaggtgggagcttcccacacctgcctgcgttctccccatgaacttacatactaggtgccttttgtttttggctttcctggttctgtctagactggcctaggggccagggctttgggatgggaggccgggagaggatgtcttttttaaggctctgtgccttggtctctccttcctcttggctgagatagcagaggggctccccgggtctctcactgttgcagtggcctggccgttcagcctgtctcccccaacaccccgcctgcctcctggctcaggcccagcttattgtgtgcgctgcctggccaggccctgggtcttgccatgtgctgggtggtagatttcctcctcccagtgccttctgggaagggagagggcctctgcctgggacactgcgggacagagggtggctggagtgaattaaagcctttgttttttaaagaaatggcaaagccttcgactgacccttgaccccctgctccctcagcagagacggagggaggggctgctggtggtcagggacctgcactgtgtagagggagcctggctgtgtggcctggaacaagtccctccctccctgtgcgcctcaggtggcctgtctgtgagatgagaagaagaccagactgaagcctgttcaccatatgccaggcagtgctttct

SEQ ID NO: 9 - protein, 173 aa, Homo sapiens
MALRVVRSVRALLCTLRAVPSPAAPCPPRPWQLGVGAVRTLRTGPALLSVRKFTEKHEWVTTENGIGTVGISNFAQEALGDVVYCSLPEVGTKLNKQDEFGALESVKAASELYSPLSGEVTEINEALAENPGLVNKSCYEDGWLIKMTLSNPSELDELMSEEAYEKYIKSIEE

SEQ ID NO: 10 - DNA, 1538 bp, Homo sapiens
cagccggctccctccggccgcgaactgcccctccccgccccgcctcccggcgcgggtggccgaggcgtagcgctgcgacccccgcacccctgcgaacatggcgctgcgagtggtgcggagcgtgcgggccctgctctgcaccctgcgcgcggtcccgtcacccgccgcgccctgcccgccgaggccctggcagctgggggtgggcgccgtccgtacgctgcgcactggacccgctctgctctcggtgcgtaaattcacagagaaacacgaatgggtaacaacagaaaatggcattggaacagtgggaatcagcaattttgcacaggaagcgttgggagatgttgtttattgtagtctccctgaagttgggacaaaattgaacaaacaagatgagtttggtgctttggaaagtgtgaaagctgctagtgaactctattctcctttatcaggagaagtaactgaaattaatgaagctcttgcagaaaatccaggacttgtaaacaaatcttgttatgaagatggttggctgatcaagatgacactgagtaacccttcagaactagatgaacttatgagtgaagaagcatatgagaaatacataaaatctattgaggagtgaaaatggaactcctaaataaactagtatgaaataacgcaagccagcagagttgtcttaaattagtggtggatagaagacttagaatagaaacttttagtattaccgatggggaaaaaaaaactactgttaacactgctaatgaaagaaaatgccctttaactttctaatgattatagataaatataatatgcgtctttttcacaatatcctatgatttttagactaggctctagtgttcagaattcatgaaattatccatggtaaaaactagttataaaaattacataattcaaagataacattgttattcttaagccttatataatattgtaacttgcatgtatccatacctggatttgggatgaaatacttaatgatctttccattggaaataactggaagtgaagaggttttgttgcttgtacagtgtcagatgaggaacaccactatcttaattttgcgatacactgcatttgctggtgctatttttatacagtgaagcaacagctttgcagcaaaataataaaatacttcttcgttaatcatgtttgttttgatgttaatatttcatttagtaactctgctagtatttgtgaaagtgctaactttaacttacggaaagttactttttaaaaggaaatttaagccagaacaatgcaaagctccaagaaaatgttttctttagtcacaaatctggtttttcttaagccaagatctgtcacctttaacataataaaaaataaatcaccaactttgattttctatcatgcgaggtctgaagaaagaagaggaaagacagaggaaggtggaagttttgatcagtatagcacatggtgtttttaagttgttaaaccacgttcaggtttccacttaagtcatgggaataaaagtggacaaggactgaagctttatgagctca

SEQ ID NO: 11 - protein, 1020 aa, Homo sapiens
MQSCARAWGLRLGRGVGGGRRLAGGSGPCWAPRSRDSSSGGGDSAAAGASRLLERLLPRHDDFARRHIGPGDKDQREMLQTLGLASIDELIEKTVPANIRLKRPLKMEDPVCENEILATLHAISSKNQIWRSYIGMGYYNCSVPQTILRNLLENSGWITQYTPYQPEVSQGRLESLLNYQTMVCDITGLDMANASLLDEGTAAAEALQLCYRHNKRRKFLVDPRCHPQTIAVVQTRAKYTGVLTELKLPCEMDFSGKDVSGVLFQYPDTEGKVEDFTELVERAHQSGSLACCATDLLALCILRPPGEFGVDIALGSSQRFGVPLGYGGPHAAFFAVRESLVRMMPGRMVGVTRDATGKEVYRLALQTREQHIRRDKATSNICTAQALLANMAAMFAIYHGSHGLEHIARRVHNATLILSEGLKRAGHQLQHDLFFDTLKIQCGCSVKEVLGRAAQRQINFRLFEDGTLGISLDETVNEKDLDDLLWIFGCESSAELVAESMGEECRGIPGSVFKRTSPFLTHQVFNSYHSETNIVRYMKKLENKDISLVHSMIPLGSCTMKLNSSSELAPITWKEFANIHPFVPLDQAQGYQQLFRELEKDLCELTGYDQVCFQPNSGAQGEYAGLATIRAYLNQKGEGHRTVCLIPKSAHGTNPASAHMAGMKIQPVEVDKYGNIDAVHLKAMVDKHKENLAAIMITYPSTNGVFEENISDVCDLIHQHGGQVYLDGANMNAQVGICRPGDFGSDVSHLNLHKTFCIPHGGGGPGMGPIGVKKHLAPFLPNHPVISLKRNEDACPVGTVSAAPWGSSSILPISWAYIKMMGGKGLKQATETAILNANYMAKRLETHYRILFRGARGYVGHEFILDTRPFKKSANIEAVDVAKRLQDYGFHAPTMSWPVAGTLMVEPTESEDKAELDRFCDAMISIRQEIADIEEGRIDPRVNPLKMSPHSLTCVTSSHWDRPYSREVAAFPLPFVKPENKFWPTIARIDDIYGDQHLVCTCPPMEVYESPFSEQKRASS

SEQ ID NO: 12 - DNA, 3820 bp, Homo sapiens
ctttgcgcgagtgtcttggttgagcgcagcgcccattcattgcccgcgagcgtccatccatctgtccggccgactgtccagcgaaaggggctccaggccgggcgcagccgccacccgggggaccgaggccaggagaggggccaagagcgcggctgacccttgcgggccggggcaggggacggtggccgcggccatgcagtcctgtgccagggcgtgggggctgcgcctgggccgcggggtcgggggcggccgccgcctggctgggggatcggggccgtgctgggcgccgcggagccgggacagcagcagtggcggcggggacagcgccgcggctggggcctcgcgcctcctggagcgccttctgcccagacacgacgacttcgctcggaggcacatcggccctggggacaaagaccagagagagatgctgcagaccttggggctggcgagcattgatgaattgatcgagaagacggtccctgccaacatccgtttgaaaagacccttgaaaatggaagaccctgtttgtgaaaatgaaatccttgcaactctgcatgccatttcaagcaaaaaccagatctggagatcgtatattggcatgggctattataactgctcagtgccacagacgattttgcggaacttactggagaactcaggatggatcacccagtatactccataccagcctgaggtgtctcaggggaggctggagagtttactcaactaccagaccatggtgtgtgacatcacaggcctggacatggccaatgcatccctgctggatgaggggactgcagccgcagaggcactgcagctgtgctacagacacaacaagaggaggaaatttctcgttgatccccgttgccacccacagacaatagctgttgtccagactcgagccaaatatactggagtcctcactgagctgaagttaccctgtgaaatggacttcagtggaaaagatgtcagtggagtgttgttccagtacccagacacggaggggaaggtggaagactttacggaactcgtggagagagctcatcagagtgggagcctggcctgctgtgctactgaccttttagctttgtgcatcttgaggccacctggagaatttggggtagacatcgccctgggcagctcccagagatttggagtgccactgggctatgggggaccccatgcagcattttttgctgtccgagaaagcttggtgagaatgatgcctggaagaatggtgggggtaacaagagatgccactgggaaagaagtgtatcgtcttgctcttcaaaccagggagcaacacattcggagagacaaggctaccagcaacatctgtacagctcaggccctcttggcgaatatggctgccatgtttgcaatctaccatggttcccatgggctggagcatattgctaggagggtacataatgccactttgattttgtcagaaggtctcaagcgagcagggcatcaactccagcatgacctgttctttgataccttgaagattcagtgtggctgctcagtgaaggaggtcttgggcagggccgctcagcggcagatcaattttcggctttttgaggatggcacacttggtatttctcttgatgaaacagtcaatgaaaaagatctggacgatttgttgtggatctttggttgtgagtcatctgcagaactggttgctgaaagcatgggagaggagtgcagaggtattccagggtctgtgttcaagaggaccagcccgttcctcacccatcaagtgttcaacagctaccactctgaaacaaacattgtccggtacatgaagaaactggaaaataaagacatttcccttgttcacagcatgattccactgggatcctgcaccatgaaactgaacagttcgtctgaactcgcacctatcacatggaaagaatttgcaaacatccacccctttgtgcctctggatcaagctcaaggatatcagcagcttttccgagagcttgagaaggatttgtgtgaactcacaggttatgaccaggtctgtttccagccaaacagcggagcccagggagaatatgctggactggccactatccgagcctacttaaaccagaaaggagaggggcacagaacggtttgcctcattccgaaatcagcacatgggaccaacccagcaagtgcccacatggcaggcatgaagattcagcctgtggaggtggataaatatgggaatatcgatgcagttcacctcaaggccatggtggataagcacaaggagaacctagcagctatcatgattacatacccatccaccaatggggtgtttgaagagaacatcagtgacgtgtgtgacctcatccatcaacatggaggacaggtctacctagacggggcaaatatgaatgctcaggtgggaatctgtcgccctggagacttcgggtctgatgtctcgcacctaaatcttcacaagaccttctgcattccccacggaggaggtggtcctggcatggggcccatcggagtgaagaaacatctcgccccgtttttgcccaatcatcccgtcatttcactaaagcggaatgaggatgcctgtcctgtgggaaccgtcagtgcggccccatggggctccagttccatcttgcccatttcctgggcttatatcaagatgatgggaggcaagggtcttaaacaagccacggaaactgcgatattaaatgccaactacatggccaagcgattagaaacacactacagaattcttttcaggggtgcaagaggttatgtgggtcatgaatttattttggacacgagacccttcaaaaagtctgcaaatattgaggctgtggatgtggccaagagactccaggattatggatttcacgcccctaccatgtcctggcctgtggcagggaccctcatggtggagcccactgagtcggaggacaaggcagagctggacagattctgtgatgccatgatcagcattcggcaggaaattgctgacattgaggagggccgcatcgaccccagggtcaatccgctgaagatgtctccacactccctgacctgcgttacatcttcccactgggaccggccttattccagagaggtggcagcattcccactccccttcgtgaaaccagagaacaaattctggccaacgattgcccggattgatgacatatatggagatcagcacctggtttgtacctgcccacccatggaagtttatgagtctccattttctgaacaaaagagggcgtcttcttagtcctctgtccctaagtttaaaggactgatttgatgcctctccccagagcatttgataagcaagaaagatttcatctcccaccccagcctcaagtaggagttttatatactgtgtatatctctgtaatctctgtcaaggtaaatgtaaatacagtagctggagggagtcgaagctgatggttggaagacggatttgctttggtattctgcttccacatgtgccagttgcctggattgggagccattttgtgttttgcgtagaaagttttaggaactttaacttttaatgtggcaagtttgcagatgtcatagaggctatcctggagacttaatagacatttttttgttccaaaagagtccatgtggactgtgccatctgtgggaaatcccagggcaaatgtttacattttgtataccctgaagaactctttttcctctaatatgcctaatctgtaatcacatttctgagtgttctcctctttttctgtgtgaggtttttttttttttaatctgcatttattagtattctaataaaagcatcttgatcggaagaaaaaaaaaaaaa

SEQ ID NO: 13 - protein, 935 aa, Homo sapiens [sequence omitted]

SEQ ID NO: 14 - DNA, 3466 bp, Homo sapiens
aattacggccggattccggagtcctttccagctccctcttcggccgggtttcccgccgaatacaaaggcgcactgtgaactggctctttctttccgccaatcatttccgccagccattcatcaccgattttcttcatcttcccctccctcttccgtcccgcagtccccgacctgttagctctcggttagttaagggactcgggtccttccgaactgcgcatgcgccaccgcgtctgcagggggagaagcgggcaggggcgcaggcgcagtagtgtgatcccctggccagtccctaagcacgtgggttgggttgtcctgcttggctgcggagggagtggaacctcgatattggtggtgtccatcgtgggcagcggactaataaaggccatggcgccagcagaaatcctgaacgggaaggagatctccgcgcaaataagggcgagactgaaaaatcaagtcactcagttgaaggagcaagtacctggtttcacaccacgcctggcaatattacaggttggcaacagagatgattccaatctttatataaatgtgaagctgaaggctgctgaagagattgggatcaaagccactcacattaagttaccaagaacaaccacagaatctgaggtgatgaagtacattacatctttgaatgaagactctactgtacatgggttcttagtgcagctacctttagattcagagaattccattaacactgaagaagtgatcaatgctattgcacccgagaaggatgtggatggattgactagcatcaatgctgggaaacttgctagaggtgacctcaatgactgtttcattccttgtacgcctaagggatgcttggaactcatcaaagagacaggggtgccgattgccggaaggcatgctgtggtggttgggcgcagtaaaatagttggggccccgatgcatgacttgcttctgtggaacaatgccacagtgaccacctgccactccaagactgcccatctggatgaggaggtaaataaaggtgacatcctggtggttgcaactggtcagcctgaaatggttaaaggggagtggatcaaacctggggcaatagtcatcgactgtggaatcaattatgtcccagatgataaaaaaccaaatgggagaaaagttgtgggtgatgtggcatacgacgaggccaaagagagggcgagcttcatcactcctgttcctggcggcgtagggcccatgacagttgcaatgctcatgcagagcacagtagagagtgccaagcgtttcctggagaaatttaagccaggaaagtggatgattcagtataacaaccttaacctcaagacacctgttccaagtgacattgatatatcacgatcttgtaaaccgaagcccattggtaagctggctcgagaaattggtctgctgtctgaagaggtagaattatatggtgaaacaaaggccaaagttctgctgtcagcactagaacgcctgaagcaccggcctgatgggaaatacgtggtggtgactggaataactccaacacccctgggagaagggaaaagcacaactacaatcgggctagtgcaagcccttggtgcccatctctaccagaatgtctttgcgtgtgtgcgacagccttctcagggccccacctttggaataaaaggtggcgctgcaggaggcggctactcccaggtcattcctatggaagagtttaatctccacctcacaggtgacatccatgccatcactgcagctaataacctcgttgctgcggccattgatgctcggatatttcatgaactgacccagacagacaaggctctctttaatcgtttggtgccatcagtaaatggagtgagaaggttctctgacatccaaatccgaaggttaaagagactaggcattgaaaagactgaccctaccacactgacagatgaagagataaacagatttgcaagattggacattgatccagaaaccataacttggcaaagagtgttggataccaatgatagattcctgaggaagatcacgattggacaggctccaacggagaagggtcacacacggacggcccagtttgatatctctgtggccagtgaaattatggctgtcctggctctcaccacttctctagaagacatgagagagagactgggcaaaatggtggtggcatccagtaagaaaggagagcccgtcagtgccgaagatctgggggtgagtggtgcactgacagtgcttatgaaggacgcaatcaagcccaatctcatgcagacactggagggcactccagtgtttgtccatgctggcccgtttgccaacatcgcacatggcaattcctccatcattgcagaccggatcgcactcaagcttgttggcccagaagggtttgtagtgacggaagcaggatttggagcagacattggaatggaaaagttttttaacatcaaatgccggtattccggcctctgcccccacgtggtggtgcttgttgccactgtcagggctctcaagatgcacgggggcggccccacggtcactgctggactgcctcttcccaaggcttacatacaggagaacctggagctggttgaaaaaggcttcagtaacttgaagaaacaaattgaaaatgccagaatgtttggaattccagtagtagtggccgtgaatgcattcaagacggatacagagtctgagctggacctcatcagccgcctttccagagaacatggggcttttgatgccgtgaagtgcactcactgggcagaagggggcaagggtgccttagccctggctcaggccgtccagagagcagcacaagcacccagcagcttccagctcctttatgacctcaagctcccagttgaggataaaatcaggatcattgcacagaagatctatggagcagatgacattgaattacttcccgaagctcaacacaaagctgaagtctacacgaagcagggctttgggaatctccccatctgcatggctaaaacacacttgtctttgtctcacaacccagagcaaaaaggtgtccctacaggcttcattctgcccattcgcgacatccgcgccagcgttggggctggttttctgtaccccttagtaggaacgatgagcacaatgcctggactccccacccggccctgtttttatgatattgatttggaccctgaaacagaacaggtgaatggattattctaaacagatcaccatccatcttcaagaagctactttgaaagtctggccagtgtctattcaggcccactgggagttaggaagtataagtaagccaagagaagtcagcccctgcccagaagatctgaaactaatagtaggagtttccccagaagtcattttcagccttaattctcatcatgtataaattaacataaatcatgcatgtctgtttactttagtgacgttccacagaataaaaggaaacaagtttgccatcaaaaaaaaaaaaaaaaaa

SEQ ID NO: 15 - protein, 979 aa, Homo sapiens
MGTRLPLVLRQLRRPPQPPGPPRRLRVPCRASSGGGGGGGGGREGLLGQRRPQDGQARSSCSPGGRTPAARDSIVREVIQNSKEVLSLLQEKNPAFKPVLAIIQAGDDNLMQEINQNLAEEAGLNITHICLPPDSSEAEIIDEILKINEDTRVHGLALQISENLFSNKVLNALKPEKDVDGVTDINLGKLVRGDAHECFVSPVAKAVIELLEKSVGVNLDGKKILVVGAHGSLEAALQCLFQRKGSMTMSIQWKTRQLQSKLHEADIVVLGSPKPEEIPLTWIQPGTTVLNCSHDFLSGKVGCGSPRIHFGGLIEEDDVILLAAALRIQNMVSSGRRWLREQQHRRWRLHCLKLQPLSPVPSDIEISRGQTPKAVDVLAKEIGLLADEIEIYGKSKAKVRLSVLERLKDQADGKYVLVAGITPTPLGEGKSTVTIGLVQALTAHLNVNSFACLRQPSQGPTFGVKGGAAGGGYAQVIPMEEFNLHLTGDIHAITAANNLLAAAIDTRILHENTQTDKALYNRLVPLVNGVREFSEIQLARLKKLGINKTDPSTLTEEEVSKFARLDIDPSTITWQRVLDTNDRFLRKITIGQGNTEKGHYRQAQFDIAVASEIMAVLALTDSLADMKARLGRMVVASDKSGQPVTADDLGVTGALTVLMKDAIKPNLMQTLEGTPVFVHAGPFANIAHGNSSVLADKIALKLVGEEGFVVTEAGFGADIGMEKFFNIKCRASGLVPNVVVLVATVRALKMHGGGPSVTAGVPLKKEYTEENIQLVADGCCNLQKQIQITQLFGVPVVVALNVFKTDTRAEIDLVCELAKRAGAFDAVPCYHWSVGGKGSVDLARAVREAASKRSRFQFLYDVQVPIVDKIRTIAQAVYGAKDIELSPEAQAKIDRYTQQGFGNLPICMAKTHLSLSHQPDKKGVPRDFILPISDVRASIGAGFIYPLVGTMSTMPGLPTRPCFYDIDLDTETEQVKGLF

SEQ ID NO: 16 - DNA, 3490 bp, Homo sapiens
cccctaggggcccctgggacgaggaggaagcgccaggtccttcccgccgccgccgccgccgccgccgcctgctcccctggcacgcgccccgccgccctcggcagccgcagctccgtgtcccctgagaaccagccgtcccgcgccatgggcacgcgtctgccgctcgtcctgcgccagctccgccgcccgccccagcccccgggccctccgcgccgcctccgtgtgccctgtcgcgctagcagcggcggcggcggaggcggcggcggtggccgggagggcctgcttggacagcggcggccgcaggatggccaggcccggagcagctgcagccccggcggccgaacgcccgcggcgcgggactccatcgtcagagaagtcattcagaattcaaaagaagttctaagtttattgcaagaaaaaaaccctgccttcaagccggttcttgcaattatccaggcaggtgacgacaacttgatgcaggaaatcaaccagaatttggctgaggaggctggtctgaacatcactcacatttgcctccctccagatagcagtgaagccgagattatagatgaaatcttaaagatcaatgaagataccagagtacatggccttgcccttcagatctctgagaacttgtttagcaacaaagtcctcaatgccttgaaaccagaaaaagatgtggatggagtaacagacataaacctggggaagctggtgcgaggggatgcccatgaatgttttgtttcacctgttgccaaagctgtaattgaacttcttgaaaaatcagtaggtgtcaacctagatggaaagaagattttggtagtgggggcccatgggtctttggaagctgctctacaatgcctgttccagagaaaagggtccatgacaatgagcatccagtggaaaacacgccagcttcaaagcaagcttcacgaggctgacattgtggtcctaggctcacctaagccagaagagattccccttacttggatacaaccaggaactactgttctcaactgctcccatgacttcctgtcagggaaggttgggtgtggctctccaagaatacattttggtggactcattgaggaagatgatgtgattctccttgctgcagctctgcgaattcagaacatggtcagtagtggaaggagatggcttcgtgaacagcagcacaggcggtggagacttcactgcttgaaacttcagcctctctcccctgtgccaagtgacattgagatttcaagaggacaaactccaaaagctgtggatgtccttgccaaggagattggattgcttgcagatgaaattgaaatctatggcaaaagcaaagccaaagtacgtttgtccgtgctagaaaggttaaaggatcaagcagatggaaaatacgtcttagttgctgggatcacacccacccctcttggagaagggaagagcacagtcaccatcgggcttgtgcaggctctgaccgcacacctgaatgtcaactcctttgcctgcttgaggcagccttcccaaggaccgacgtttggagtgaaaggaggagccgcgggtggtggatatgcccaggtcatccccatggaggagttcaaccttcacttgactggagacatccacgccatcaccgctgccaataacttgctggctgccgccatcgacacgaggattcttcatgaaaacacgcaaacagataaggctctgtataatcggctggttcctttagtgaatggtgtcagagaattttcagaaattcagcttgctcggctaaaaaaactgggaataaataagactgatccgagcacactgacagaagaggaagtgagtaaatttgcccgtctcgacatcgacccatctaccatcacgtggcagagagtattggatacaaatgaccgatttctacgaaaaataaccatcgggcagggaaacacagagaagggccattaccggcaggcgcagtttgacatcgcagtggccagcgagatcatggcggtgctggccctgacggacagcctcgcagacatgaaggcacggctgggaaggatggtggtggccagtgacaaaagcgggcagcctgtgacagcagatgatttgggggtgacaggtgctttgacagttttgatgaaagatgcaataaaaccaaacctgatgcagaccctggaagggacacctgtgttcgtgcatgcgggcccttttgctaacattgctcacggcaactcttcagtgttggctgataaaattgccctgaaactggttggtgaagaaggatttgtagtgaccgaagctggctttggtgctgacatcggaatggagaaattcttcaacatcaagtgccgagcttccggcttggtgcccaacgtggttgtgttagtggcaacggtgcgagctctgaagatgcatggaggcgggccaagtgtaacggctggtgttcctcttaagaaagaatatacagaggagaacatccagctggtggcagacggctgctgtaacctccagaagcaaattcagatcactcagctctttggggttcccgttgtggtggctctgaatgtcttcaagaccgacacccgcgctgagattgacttggtgtgtgagcttgcaaagcgggctggtgcctttgatgcagtcccctgctatcactggtccgttggtggaaaaggatcggtggacttggctcgggctgtgagagaggctgcgagtaaaagaagccgattccagttcctgtatgatgttcaggttccaattgtggacaagataaggaccattgctcaggctgtctatggagccaaagatattgaactctctcctgaggcacaagccaaaatagatcgttacactcaacagggttttggaaatttgcccatctgcatggcaaagacccacctttctctatctcaccaacctgacaaaaaaggtgtgccaagggacttcatcttacctatcagtgacgtccgggccagcataggcgctgggttcatttaccctttggtcggaacgatgagcaccatgccaggactgcccacccggccctgcttttatgacatagatcttgataccgaaacagaacaagttaaaggcttgttctaagtggacaaggctctcacaggacccgatgcagactcctgaaacagactactctttgcctttttgctgcagttggagaagaaactgaatttgaaaaatgtctgttatgcaatgctggagacatggtgaaataggccaaagatttcttcttcgttcaagatgaattctgttcacagtggagtatggtgttcggcaaaaggacctccaccaagactgaaagaaactaatttatttctgtttctgtggagtttccattatttctactgcttacactttagaatgtttattttatggggactaagggattaggagtgtgaactaaaaggtaacattttccactctcaagttttctactttgtctttgaactgaaaataaacatggatctagaaaaccaaaaaaaaaaaaaaa

SEQ ID NO: 17 - protein, 350 aa, Homo sapiens
MAATSLMSALAARLLQPAHSCSLRLRPFHLAAVRNEAVVISGRKLAQQIKQEVRQEVEEWVASGNKRPHLSVILVGENPASHSYVLNKTRAAAVVGINSETIMKPASISEEELLNLINKLNNDDNVDGLLVQLPLPEHIDERRICNAVSPDKDVDGFHVINVGRMCLDQYSMLPATPWGVWEIIKRTGIPTLGKNVVVAGRSKNVGMPIAMLLHTDGAHERPGGDATVTISHRYTPKEQLKKHTILADIVISAAGIPNLITADMIKEGAAVIDVGINRVHDPVTAKPKLVGDVDFEGVRQKAGYITPVPGGVGPMTVAMLMKNTIIAAKKVLRLEEREVLKSKELGVATN

SEQ ID NO: 18 - DNA, 2208 bp, Homo sapiens
ggggcctgccacgaggccgcagtataaccgcgtggcccgcgcgcgcgcttccctcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccaaaccttttgagttcaactgatcaaaccaaaggaaaagtgttgctagagaaaattagggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggtatgaatttctgaaaaaaaaaaaaaaaaaaaaaaa

SEQ ID NO: 19 - protein, 347 aa, Homo sapiens
MTVPVRGFSLLRGRLGRAPALGRSTAPSVRAPGEPGSAFRGFRSSGVRHEAIIISGTEMAKHIQKEIQRGVESWVSLGNRRPHLSIILVGDNPASHTYVRNKIRAASAVGICSELILKPKDVSQEELLDVTDQLNMDPRVSGILVQLPLPDHVDERTICNGIAPEKDVDGFHIINIGRLCLDQHSLIPATASAVWEIIKRTGIQTFGKNVVVAGRSKNVGMPIAMLLHTDGEHERPGGDATVTIAHRYTPKEQLKIHTQLADIIIVAAGIPKLITSDMVKEGAAVIDVGINYVHDPVTGKTKLVGDVDFEAVKKKAGFITPVPGGVGPMTVAMLLKNTLLAAKKIIY

SEQ ID NO: 20 - DNA, 2372 bp, Homo sapiens
cagtccggaagccggggatccgcggccatgacggtgccggtccgcggcttctcgctgctccgcggccgccttggccgagcgccggcgttgggcagaagcacagcaccctccgtaagggcaccgggagagcccgggagtgcgttccggggctttcggagcagcggtgtgagacatgaagccattattatatcaggaaccgaaatggccaagcatatccagaaagaaatacagcgaggtgtggaatcatgggtttcccttggaaacagaagacctcacctcagtataattttagtgggagataacccagcaagccatacatatgtcaggaataagataagagctgcctctgctgtaggtatttgtagtgagctcattctaaaacctaaggatgtttctcaggaagaacttttggacgtaactgatcaattgaatatggacccaagagtcagcggtatattagttcagttaccactaccagaccacgttgatgagcgaacaatatgcaatggaattgccccagaaaaagatgtagatggatttcatattatcaatattggaagattgtgccttgatcagcattctctcatacctgccactgccagtgctgtttgggaaataataaaaagaacaggaattcaaacatttggaaaaaatgtggttgtggctggaagatccaagaacgtagggatgcctattgccatgcttttacacactgatggagagcatgaacggccaggaggtgatgcaactgtgacaatagctcacagatacacccccaaagagcaactgaagattcatacgcagctggcagatattatcatagttgctgcaggtattccaaagttgattacgtctgatatggttaaagaaggtgctgctgtaattgatgtgggtatcaactatgtccacgatccagtgacaggaaagacaaaattagttggagatgtggacttcgaagctgttaaaaagaaagctggctttatcactccagttccaggaggtgtgggacccatgacagtggcaatgcttctgaagaacacccttctggcagctaaaaaaatcatttactagatcacatgaaaggataaagcaaactgaagtcatgctatttgtttatttgacaaagggtaaaacctttatattttactacaaagctatttatttctacatggtatttattttttcatgggtgaaatcattgtgaatcaattgattcacatagttttatgcatttcctgctaatttattttgagttttaagaaaacaaccaaaacaattccaatgaaaattttagtaacaattgtttattttgagggtatttgttcataacattaaaacaataaagggctcataataaataaatatatttttgacacaattaaatattacatagagtatgtttacaacaaatatcctgtcagccaaatggttacccatataaaatgtaatttaggttttgctacttgcatgctaacatttttaatgtattttatgatctatgtcatatattaaaaaagagcttgcttactacaagaaaaatattgaaatattgaaaatattgaaaatattattattgaaaataaaatcttgatctcaactatcccccaaatgcatcctataagtccatcctaatgagaaatgatgttctatttaaggaaaggaaaatattccgggaaggcaaaaaatgcagtgctgtttggaagtgtaatgattttatcacatggtgaatgactactaagagtaatgattatatcacattgtgaatgactacttgccacagtaaaaatacatgaagaatgtgttaggtttaaacgtcgtttctttcttctaaaaaatatttggttagtaccttcactgagcaatagtggaaaaataaaaaaataagtaaacagaaaaaactaaagttgtattttcccacaaatatagtatgaatgaggtcatattaaagaacagcaactgttaatgtttgttcacaaattcagaaatctaataggaaaacatgatactttcaatgtgccaaaactaaaccttagtatacaactaaaaatctcctgccttcttgcctacctgtcttccctcttctgttacagaatttgttcctcaaagtagatgcaatgtttctaacacaatttaaattaggaaatatatatgaatgtcgttgaagtctattttgagactgctaaagctattaattgatactgtgtttttatgcccaaatcccagtatgtttatgtaccaataatgactcttacccagcgcatgtctttatcagtgtgtactcgtgacgatttgtgtgaaaatagacttgatgtttataattaataccattacaactgtataataaaagcaatttgaagaaaaaaaaaaaaaaa

SEQ ID NO: 21 - protein, 203 aa, Homo sapiens
MAAAAVSSAKRSLRGELKQRLRAMSAEERLRQSRVLSQKVIAHSEYQKSKRISIFLSMQDEIETEEIIKDIFQRGKICFIPRYRFQSNHMDMVRIESPEEISLLPKTSWNIPQPGEGDVREEALSTGGLDLIFMPGLGFDKHGNRLGRGKGYYDAYLKRCLQHQEVKPYTLALAFKEQICLQVPVNENDMKVDEVLYEDSSTA

SEQ ID NO: 22 - DNA, 2346 bp, Homo sapiens
agaccgaacccgagggcgcccagggcgccgagggcgggactggactcggcttgggcgtgagatggcggcggcagcggtgagcagcgccaagcggagcctgcggggagagctgaagcagcgtctgcgggcgatgagtgccgaggagcggctacgccagtcccgcgtactgagccagaaggtgattgcccacagtgagtatcaaaagtccaaaagaatttccatctttctgagcatgcaagatgaaattgagacagaagagatcatcaaggacattttccaacgaggcaaaatctgcttcatccctcggtaccggttccagagcaatcacatggatatggtgagaatagaatcaccagaggaaatttctttacttcccaaaacatcctggaatatccctcagcctggtgagggtgatgttcgggaggaggccttgtccacagggggacttgatctcatcttcatgccaggccttgggtttgacaaacatggcaaccgactggggaggggcaagggctactatgatgcctatctgaagcgctgtttgcagcatcaggaagtgaagccctacaccctggcgttggctttcaaagaacagatttgcctccaggtcccagtgaatgaaaacgacatgaaggtagatgaagtcctttacgaagactcgtcaacagcttaaatctggattactacagccaaataatcagtgttttatatgagagtaaagcaaagtatgtgtatttttcccttgtcaaaaattagttgaaattgttcattaatgtgaatacagactgcattttaaaattgtaattatgaaataccttatataaaaccatctttaaaaaccaatagaagtgtgaatagtagaatattaattaaaatggaggctatcagcctgtgattttcagcttaacttcctggtgttaatgtgacaagttgatctgtctactttgcaatttaagttaaatatttatgaggaactgtgctccgactgagtgcgagaggaaggtaaacttgcgggagtgggcactgtatttcattacgcctttcatgacctggtctgtcctggcaggcacatggagacttggggactattaatttatttgttggtatttgctttggatacagaattccctaagaatattatctcacttcatcatgacttcctttacccactctagaattttatgttggacactttgtagcttttggtggttagtggagggaaaccttttatgatttaaatacttttactccactgattggttaccatcactgcatgtatcagcccttgatgagtttaagatctagtatcttataagttagaaattatttctgtttactcatggtttctgctttggaaatgaaatttgctgtgagttgaaagttggcagatggcaacacagctagggagcaataattttgttgtggggaggatttggtcatctccagaaacccgggagtcacgtggctctcttactcacactccatgtccatcctgtcaccaggtcttgttgattttgcctctgaaatgcctcttaaatctattctctcctttcaattttcctgtcactccttttgtccagccttttagcatttctaagctggactaggcgagagtgtcacacctgcttcccttggcttccatcttgcctcttccagtttatttctcaagctgcagtcaaactgatcttagaaaacacgaatctaatcatgcagcacccctgactaaggtcttccggtggctgttcagagcctcttgggtagcaaacagatggcttctgttgtatacaaagccccttaagagaggtctcctcatctactttttctagcctcttctctcccaactctgtattctcctgtaacactgactgagcactgcaggcttctgccctttgcacatagtaagcatgcatttctctctgtctgaaatgctctttctgttgttcatctagaagactgttttcccttgaagactcagccctagcatcacctcttctgtgaagtcttctgctactttcccaagcagagtgagtgttccttcccttgtctgagtggccttggccattgatgtgcttatcatgttgtcttacgtatcaaattatttgttgtcatatctgtccccttcaccatactgtgagctccaaagaaaagaagtgatctttatacttcttatgcttagtacacaactagacatatagtgtgtgtgtgagagagagaattttttaatgaaataattgaatacattggaagtgtttcattcaaaatactcatccattattctttggatagtagcataaatttgatgttttatgtacaaaagtaaaaacatttgaaaaataaaaaaaaaaa

SEQ ID NO: 23 - protein, 698 aa, Homo sapiens
MRRFLLLYATQQGQAKAIAEEICEQAVVHGFSADLHCISESDKYDLKTETAPLVVVVSTTGTGDPPDTARKFVKEIQNQTLPVDFFAHLRYGLLGLGDSEYTYFCNGGKIIDKRLQELGARHFYDTGHADDCVGLELVVEPWIAGLWPALRKHFRSSRGQEEISGALPVASPASSRTDLVKSELLHIESQVELLRFDDSGRKDSEVLKQNAVNSNQSNVVIEDFESSLTRSVPPLSQASLNIPGLPPEYLQVHLQESLGQEESQVSVTSADPVFQVPISKAVQLTTNDAIKTTLLVELDISNTDFSYQPGDAFSVICPNSDSEVQSLLQRLQLEDKREHCVLLKIKADTKKKGATLPQHIPAGCSLQFIFTWCLEIRAIPKKAFLRALVDYTSDSAEKRRLQELCSKQGAADYSRFVRDACACLLDLLLAFPSCQPPLSLLLEHLPKLQPRPYSCASSSLFHPGKLHFVFNIVEFLSTATTEVLRKGVCTGWLALLVASVLQPNIHASHEDSGKALAPKISISPRTTNSFHLPDDPSIPIIMVGPGTGIAPFIGFLQHREKLQEQHPDGNFGAMWLFFGCRHKDRDYLFRKELRHFLKHGILTHLKVSFSRDAPVGEEEAPAKYVQDNIQLHGQQVARILLQENGHIYVCGDAKNMAKDVHDALVQIISKEVGVEKLEAMKTLATLKEEKRYLQDIWS

SEQ ID NO: 24 - DNA, 3317 bp, Homo sapiens
ggagctttctattggtcctgggtaccgagcatgggcgctgcgtcagtgcgcgctggcgcaaggttggtggaagtcgcgttgtgcaggttcgtgcccggctggcgcggcgtggtttcactgttacatgccttgaagtgatgaggaggtttctgttactatatgctacacagcagggacaggcaaaggccatcgcagaagaaatatgtgagcaagctgtggtacatggattttctgcagatcttcactgtattagtgaatccgataagtatgacctaaaaaccgaaacagctcctcttgttgttgtggtttctaccacgggcaccggagacccacccgacacagcccgcaagtttgttaaggaaatacagaaccaaacactgccggttgatttctttgctcacctgcggtatgggttactgggtctcggtgattcagaatacacctacttttgcaatggggggaagataattgataaacgacttcaagagcttggagcccggcatttctatgacactggacatgcagatgactgtgtaggtttagaacttgtggttgagccgtggattgctggactctggccagccctcagaaagcattttaggtcaagcagaggacaagaggagataagtggcgcactcccggtggcatcacctgcatcctcgaggacagaccttgtgaagtcagagctgctacacattgaatctcaagtcgagcttctgagattcgatgattcaggaagaaaggattctgaggttttgaagcaaaatgcagtgaacagcaaccaatccaatgttgtaattgaagactttgagtcctcacttacccgttcggtacccccactctcacaagcctctctgaatattcctggtttacccccagaatatttacaggtacatctgcaggagtctcttggccaggaggaaagccaagtatctgtgacttcagcagatccagtttttcaagtgccaatttcaaaggcagttcaacttactacgaatgatgccataaaaaccactctgctggtagaattggacatttcaaatacagacttttcctatcagcctggagatgccttcagcgtgatctgccctaacagtgattctgaggtacaaagcctactccaaagactgcagcttgaagataaaagagagcactgcgtccttttgaaaataaaggcagacacaaagaagaaaggagctaccttaccccagcatatacctgcgggatgttctctccagttcatttttacctggtgtcttgaaatccgagcaattcctaaaaaggcatttttgcgagcccttgtggactataccagtgacagtgctgaaaagcgcaggctacaggagctgtgcagtaaacaaggggcagccgattatagccgctttgtacgagatgcctgtgcctgcttgttggatctcctcctcgctttcccttcttgccagccaccactcagtctcctgctcgaacatcttcctaaacttcaacccagaccatattcgtgtgcaagctcaagtttatttcacccaggaaagctccattttgtcttcaacattgtggaatttctgtctactgccacaacagaggttctgcggaagggagtatgtacaggctggctggccttgttggttgcttcagttcttcagccaaacatacatgcatcccatgaagacagcgggaaagccctggctcctaagatatccatctctcctcgaacaacaaattctttccacttaccagatgacccctcaatccccatcataatggtgggtccaggaaccggcatagccccgtttattgggttcctacaacatagagagaaactccaagaacaacacccagatggaaattttggagcaatgtggttgttttttggctgcaggcataaggatagggattatctattcagaaaagagctcagacatttccttaagcatgggatcttaactcatctaaaggtttccttctcaagagatgctcctgttggggaggaggaagccccagcaaagtatgtgcaagacaacatccagcttcatggccagcaggtggcgagaatcctcctccaggagaacggccatatttatgtgtgtggagatgcaaagaatatggccaaggatgtacatgatgcccttgtgcaaataataagcaaagaggttggagttgaaaaactagaagcaatgaaaaccctggccactttaaaagaagaaaaacgctaccttcaggatatttggtcataaaaccagaaattaaagaaagaggattaagcttttttgactgaaagtactaaaagtcagctttactagtgccaaacctttaaattttcaaaagaaaattttctttcaacatttcttgaaggacatggagtggagattggatcatttaacaatataacaaaacttcctgatttgattttacgtatcttctatctacgcccttcctgtgcctgtgactctccccaaattgccctgttgccttgagctcttctgagctaaggcagccttcagtccctatcagcgcctcctttacttcccagagaacttcacagagactctgtccttccatgcaaaggcttcctgaaatagggagactgactgagtagctcattcttgtgacttacagtgccaacatttaaaaaagtatgaaaatgatttatttttatatgatgtatacccataaagaatgctcatattaatgtacttaaattacacatgtagagcatatctgttatatgtttatgtaactatcaaatggttatttgttactaaagctatatttctgataaaaaatattttaggataattgcctacagagggatttatttttatgatgctggaaatatgaaatgtattttaaaatttcactctggcatatgatttatctatcaccattacttttttttaagtcacaatttcagaattttgggacatttgcattcaatttacaggtaccagtacgtacatattttaatagaaagatacaacctttttattttcactccttttatttctgctgcttggcacatttttgagttttcccacattatttgtctccatgataccactcaagcagtgtgctggacctaaaatactgactttagttagtatccttggatttttagattcccagtgtctaattccctgttataatttgcacaaacaaaacaaaatgttatgataatctttctccactgttctaatatatattgtatttttatttgatagcttgggatttaaaacatctctgttgaaggcttttgatccttttgagaaataaagatctgaaagaaatggcataatcttaaaaaaaaaaaaaaaaaaaaa

SEQ ID NO: 25 - protein, 483 aa, Homo sapiens
MTMPVNGAHKDADLWSSHDKMLAQPLKDSDVEVYNIIKKESNRQRVGLELIASENFASRAVLEALGSCLNNKYSEGYPGQRYYGGTEFIDELETLCQKRALQAYKLDPQCWGVNVQPYSGSPANFAVYTALVEPHGRIMGLDLPDGGHLTHGFMTDKKKISATSIFFESMPYKVNPDTGYINYDQLEENARLFHPKLIIAGTSCYSRNLEYARLRKIADENGAYLMADMAHISGLVAAGVVPSPFEHCHVVTTTTHKTLRGCRAGMIFYRKGVKSVDPKTGKEILYNLESLINSAVFPGLQGGPHNHAIAGVAVALKQAMTLEFKVYQHQVVANCRALSEALTELGYKIVTGGSDNHLILVDLRSKGTDGGRAEKVLEACSIACNKNTCPGDRSALRPSGLRLGTPALTSRGLLEKDFQKVAHFIHRGIELTLQIQSDTGVRATLKEFKERLAGDKYQAAVQALREEVESFASLFPLPGLPDF

SEQ ID NO: 26 - DNA, 2540 bp, Homo sapiens
gcctggcgcgcagagtgcaccttcctgagctcgagcggtccagcgccaagttcggggtttggggttggagcggctggtcacgtggctggcccgcggcggtgcgcggggcgttgggtcagcgggtctgggactggtggcaccggcggcggcgtaggacggaggcgtcgctaggcagcttcgaaccagtgcaatgacgatgccagtcaacggggcccacaaggatgctgacctgtggtcctcacatgacaagatgctggcacaacccctcaaagacagtgatgttgaggtttacaacatcattaagaaggagagtaaccggcagagggttggattggagctgattgcctcggagaatttcgccagccgagcagttttggaggccctaggctcttgcttaaataacaaatactctgaggggtacccgggccagagatactatggcgggactgagtttattgatgaactggagaccctctgtcagaagcgagccctgcaggcctataagctggacccacagtgctggggggtcaacgtccagccctactcaggctcccctgcaaactttgctgtgtacactgccctggtggaaccccatgggcgcatcatgggcctggaccttccggatgggggccacctgacccatgggttcatgacagacaagaagaaaatctctgccacgtccatcttctttgaatctatgccctacaaggtgaacccagatactggctacatcaactatgaccagctggaggagaacgcacgcctcttccacccgaagctgatcatcgcaggaaccagctgctactcccgaaacctggaatatgcccggctacggaagattgcagatgagaacggggcgtatctcatggcggacatggctcacatcagcgggctggtggcggctggcgtggtgccctccccatttgaacactgccatgtggtgaccaccaccactcacaagaccctgcgaggctgccgagctggcatgatcttctacaggaaaggagtgaaaagtgtggatcccaagactggcaaagagattctgtacaacctggagtctcttatcaattctgctgtgttccctggcctgcagggaggtccccacaaccacgccattgctggggttgctgtggcactgaagcaagctatgactctggaatttaaagtttatcaacaccaggtggtggccaactgcagggctctgtctgaggccctgacggagctgggctacaaaatagtcacaggtggttctgacaaccatttgatccttgtggatctccgttccaaaggcacagatggtggaagggctgagaaggtgctagaagcctgttctattgcctgcaacaagaacacctgtccaggtgacagaagcgctctgcggcccagtggactgcggctggggaccccagcactgacgtcccgtggacttttggaaaaagacttccaaaaagtagcccactttattcacagagggatagagctgaccctgcagatccagagcgacactggtgtcagagccaccctgaaagagttcaaggagagactggcaggggataagtaccaggcggccgtgcaggctctccgggaggaggttgagagcttcgcctctctcttccctctgcctggcctgcctgacttctaaaggagcgggcccactctggacccacctggcgccacagaggaagctgcctgccggaggacccccacctgagagatggatgagctgctccaaaggggaactgttgacactcgggccctttgagggggtttcttttggacttttttcatgttttcttcacaaatcaaaatttgtttaagtctcattgttagtaattctgggacaggttattaaaggatttaaatttgaacctggctttctcacagctggacataattctaggaaaataaaatactatgtcgccacttggtcataatcatttagatggtggtgtagggcaaagctgttagaaagattgtagcgttttactctccctgggctttcctccgccttgctgcaacagagaggaaatgcccatgtccacagcttgtacacactgccccctcactatcttgttatccagtggcatgccaaaggagaactgaattagcttctgaggcttctgctgtaaatcagaagtgtatgttagtcaagagtaaacaagatgcacccagtatggtgggagggttttgctgtcagtagctcaaagtatggtgtagaaatggcctcctccctccatcctgggaagtcccagtcccatcctggtgtgagaatcaaccaggctttcctgctccacctgagataaccaactccctcccgtaatcaggaagccaaatgtcaccttcccaaagaaattttattttcacgtagctgaagtgcaaaacatagatgaccatttttaataagcacaatcaaatttttaaccacagaatgtctacaagaattatagctttaaaaaatacaaccaatttttatatttcaaaaatatttgaactcaaataaattaatttcttaaaaagtaaaaaaaaaaaaaaaaaaaaaa

SEQ ID NO: 27 - protein, 504 aa, Homo sapiens
MLYFSLFWAARPLQRCGQLVRMAIRAQHSNAAQTQTGEANRGWTGQESLSDSDPEMWELLQREKDRQCRGLELIASENFCSRAALEALGSCLNNKYSEGYPGKRYYGGAEVVDEIELLCQRRALEAFDLDPAQWGVNVQPYSGSPANLAVYTALLQPHDRIMGLDLPDGGHLTHGYMSDVKRISATSIFFESMPYKLNPKTGLIDYNQLALTARLFRPRLIIAGTSAYARLIDYARMREVCDEVKAHLLADMAHISGLVAAKVIPSPFKHADIVTTTTHKTLRGARSGLIFYRKGVKAVDPKTGREIPYTFEDRINFAVFPSLQGGPHNHAIAAVAVALKQACTPMFREYSLQVLKNARAMADALLERGYSLVSGGTDNHLVLVDLRPKGLDGARAERVLELVSITANKNTCPGDRSAITPGGLRLGAPALTSRQFREDDFRRVVDFIDEGVNIGLEVKSKTAKLQDFKSFLLKDSETSQRLANLRQRVEQFARAFPMPGFDEH

SEQ ID NO: 28 - DNA, 2295 bp, Homo sapiens
ataaagaaaaaagcggtgagtgggcgaactacaattcccaaaaggccacaaaggggccaccactacgcatgcgtagatccctcccgttagctttggcgcctcagcgagctcttctcgcgcatgcgttctccgaacggtcttcttccgacagcttgctgccctagaccagagttggtggctggacctcctgcgacttccgagttgcgatgctgtacttctctttgttttgggcggctcggcctctgcagagatgtgggcagctggtcaggatggccattcgggctcagcacagcaacgcagcccagactcagactggggaagcaaacaggggctggacaggccaggagagcctgtcggacagtgatcctgagatgtgggagttgctgcagagggagaaggacaggcagtgtcgtggcctggagctcattgcctcagagaacttctgcagccgagctgcgctggaggccctggggtcctgtctgaacaacaagtactcggagggttatcctggcaagagatactatgggggagcagaggtggtggatgaaattgagctgctgtgccagcgccgggccttggaagcctttgacctggatcctgcacagtggggagtcaatgtccagccctactccgggtccccagccaacctggccgtctacacagcccttctgcaacctcacgaccggatcatggggctggacctgcccgatgggggccatctcacccacggctacatgtctgacgtcaagcggatatcagccacgtccatcttcttcgagtctatgccctataagctcaaccccaaaactggcctcattgactacaaccagctggcactgactgctcgacttttccggccacggctcatcatagctggcaccagcgcctatgctcgcctcattgactacgcccgcatgagagaggtgtgtgatgaagtcaaagcacacctgctggcagacatggcccacatcagtggcctggtggctgccaaggtgattccctcgcctttcaagcacgcggacatcgtcaccaccactactcacaagactcttcgaggggccaggtcagggctcatcttctaccggaaaggggtgaaggctgtggaccccaagactggccgggagatcccttacacatttgaggaccgaatcaactttgccgtgttcccatccctgcaggggggcccccacaatcatgccattgctgcagtagctgtggccctaaagcaggcctgcacccccatgttccgggagtactccctgcaggttctgaagaatgctcgggccatggcagatgccctgctagagcgaggctactcactggtatcaggtggtactgacaaccacctggtgctggtggacctgcggcccaagggcctggatggagctcgggctgagcgggtgctagagcttgtatccatcactgccaacaagaacacctgtcctggagaccgaagtgccatcacaccgggcggcctgcggcttggggccccagccttaacttctcgacagttccgtgaggatgacttccggagagttgtggactttatagatgaaggggtcaacattggcttagaggtgaagagcaagactgccaagctccaggatttcaaatccttcctgcttaaggactcagaaacaagtcagcgtctggccaacctcaggcaacgggtggagcagtttgccagggccttccccatgcctggttttgatgagcattgaaggcacctgggaaatgaggcccacagactcaaagttactctccttccccctacctgggccagtgaaatagaaagcctttctattttttggtgcgggagggaagacctctcacttagggcaagagccaggtatagtctcccttcccagaatttgtaactgagaagatcttttctttttcctttttttggtaacaagacttagaaggagggcccaggcactttctgtttgaacccctgtcatgatcacagtgtcagagacgcgtcctctttcttggggaagttgaggagtgcccttcagagccagtagcaggcaggggtgggtaggcaccctccttcctgtttttatctaataaaatgctaacctgccctgagtttccattactgtgggtggggttcccctgggccaaacagtgatttgtctccctcaatgtgtacaccgctccgctcccaccaccgctaccacaaggacccccggggctgcagcctcctctttctgtctctgatcagagccgacaccagacgtgattagcaggcgcagcaaattcaatttgttaaatgaaattgtattttgccca

SEQ ID NO: 29 - protein, 315 aa, Homo sapiens
MTGQGQSASGSSAWSTVFRHVRYENLIAGVSGGVLSNLALHPLDLVKIRFAVSDGLELRPKYNGILHCLTTIWKLDGLRGLYQGVTPNIWGAGLSWGLYFFFYNAIKSYKTEGRAERLEATEYLVSAAEAGAMTLCITNPLWVTKTRLMLQYDAVVNSPHRQYKGMFDTLVKIYKYEGVRGLYKGFVPGLFGTSHGALQFMAYELLKLKYNQHINRLPEAQLSTVEYISVAALSKIFAVAATYPYQVVRARLQDQHMFYSGVIDVITKTWRKEGVGGFYKGIAPNLIRVTPACCITFVVYENVSHFLLDLREKRK

SEQ ID NO: 30 - DNA, 3133 bp, Homo sapiens
actcgcggagcggcggcctgctggctcaactggatcctgcgccgctccgtagttttgccggcaaacgttagcaaggggcggttctttagctgtgcagtcgcttccgcgtccgtgggctggagcatttgtgggcgaggcagggcggagactcgggagaggctgggacctcccctccatcgcgctttccgccggcgtgacgtagtgtctgtgccccgttcttgccccctcagtactagagtctccggcttcgctcacgcgccttgggcataagagtcctctcgttggtcccggaggtggggttgcgctcacaaggggcgaccgtcgccacggtggcggccactgcatcgcgtcccacctccgcggccctgggcgccgtggtgtcgacgggccccgagcctatgacgggccagggccagtcggcgtccgggtcgtcggcgtggagcacggtattccgccacgtccggtatgagaacctgatagcgggcgtgagcggcggcgtcttatccaaccttgcgctgcatccgctcgacctcgtgaagatccgcttcgccgtgagtgatggattggaactgagaccgaaatataatggaattttacattgcttgactaccatttggaaacttgatggactacggggactttatcaaggagtaaccccaaatatatggggtgcaggtttatcctggggactctactttttcttttacaatgccatcaagtcatataaaacagaaggaagagctgaacgtttagaggcaacagaataccttgtctcagctgctgaagctggagccatgaccctctgcattacaaacccattatgggtaacaaaaactcgccttatgttacagtatgatgctgttgttaactccccacaccgacaatataaaggaatgtttgatacacttgtgaaaatatataagtatgaaggtgtgcgtggattatataagggatttgttcctgggctgtttggaacatcgcatggtgcccttcagtttatggcatatgaattgctgaagttgaagtacaaccagcatatcaatagattaccagaagcccagttgagcacagtagaatatatatctgttgcagcactatccaaaatatttgctgtcgcagcaacatacccatatcaagtcgtaagagctcgtcttcaggatcaacacatgttttacagtggtgtaatagatgtaatcacaaagacatggaggaaagaaggcgtcggtggattttacaagggaattgctcctaatttgattagagtgactccagcctgctgtattacctttgtggtatatgaaaacgtctcacattttttacttgaccttagagaaaagagaaagtaagctcaaagaggacaattccagtatatctgcccaaggcagcaacaagctcttttgtgtttaaggcataaaagaagaattctgcatagaaacatggctcatattcgaaattgctctatagtcattagaagccagagaactgctaagtctcctgcaatgtttttcttgctttttgccttccccatatatatggaacttggctacctctgcctgaaatggctgccatcaacacaatgttaaaactgacacgaaggatagagtttcacagatttctacgttttattggtggaagctgatttgcaacatttgctaaatggattagatgaatgtacttctttttgtgagcttacttgcctggattgctttaaaattaacctttgtgcaataccaagaaaatagctctttaaaagaatgtctttgtatgtctcaaggtaaattaaggatttactgaataaggtgttgaccaaatccagaccattttattttatttttttatttatttattttttgagatggagtcttgctttgtcgcccaggctggagtgcagtggcgtgatctcagctcactgcaacctccacctcccgggttcacgccattctcctgcctcagcctcctgagtagctgggactacaggcacctgccaccacgcctggctaacttttttttatattttgagtagaaatggggtttcaccatgttagccaggatggtctcaatctcctgaccttgtgatccgcctgccttggcctcccaaagtgctgggattacaggcgtgagccactgcgcctggccagaccattttagaattgggaaattttagtgagaaaaaatgcactgtaaatatgctttagttttaattcagttgggatgcactacctagcgaaaattgagaaactatatacttctcagagaaatatctgacatctattgtcattccattgctattttttttccccagagacttccataatttaaaataaaatcctagatccagttcttgttttttggcataaatacttaatctattttaaatttataaaatctgagcttctaggatccagctgtgtcaacctttatttagcatatataactataaatcacttattacagatgctaaatagatcaccttttacagatgctgaaatgtttgggatatgtttgttgacaaggtaaatggaaatgagaaactttatacttcagttttcagatatatggatctagatcccaaataaatgattaatcttcattggtttctcaaattcaggttgaaatacaaattaatagcctttattgattttacttttatgagtcattgtagacatctataaatataaaagggcctgtacccaaaggatgccagaatactagtatttttatttatcgtaaacatccacgagtgctgttgcactaccatctatttgttgtaaataaaagtgttgttttcaaagccatctttaaatagttctttaaaaataggtcttttttttatattttggaaaaggcattgtttttaaagtaaagataaaatggtaagtacctaattgtatttactgtaatatcttataacatgcagatgaatgctttataagttaaatatgatgtattttttcatacttctggattatactataattcatatgaaatcttgatattagtccccacacggaaaaagtgaactgcagttgatatttggtgtttaagatagcaccattgtttaaataccgcctatgtactcccaaatgaataaaacataattcttgtcctctgagagcataaaaaaaaaaaaaaa

(19) United States
(12) Patent Application Publication
(10) Pub. No.: US 2017/0073753 A1
(43) Pub. Date: Mar. 16, 2017

(54) METHODS AND KITS FOR TREATING AND CLASSIFYING INDIVIDUALS

(71) Applicant: COURTAGEN LIFE SCIENCES, INC., Woburn, MA (US)

(72) Inventor: Richard Boles, Los Angeles, CA (US)

(21) Appl. No.: 14/895,562

(22) PCT Filed: Jun. 11, 2014
(86) PCT No.: PCT/US14/41892

§ 371 Date: Dec. 3, 2015

Related U.S. Application Data
(60) Provisional application No. 61/833,906, filed on Jun. 11, 2013.

(51) Int. Cl. C12Q 1/68; A61K 31/198; A61K 31/519

(52) U.S. Cl. CPC C12Q 1/6883; A61K 31/519; A61K 31/198; C12Q 2600/112; C12Q 2600/156; C12Q 2600/106

(57) ABSTRACT

The present disclosure provides methods and kits for treating and classifying individuals at risk of or suffering from a neurological and/or mitochondrial dysfunction or disorder. In general, the individuals are treated and/or classified based on the presence of a loss-of-function mutation in nuclear DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32. Treatment involves the administration of a therapeutically effective amount of folinic acid, glycine or a pharmaceutically acceptable salt thereof.

### BACKGROUND

Neurological dysfunctions and disorders continue to be a major health threat in the population. Neurological dysfunctions and disorders occur due to dysfunction of the neurons in the central nervous system as well as the peripheral nervous system.

One frequent contributing factor of neurological dysfunctions and disorders is mitochondrial disease. Some mitochondrial diseases are due to mutations or deletions in the mitochondrial genome. Mitochondria divide and proliferate with a faster turnover rate than their host cells, and their replication is under control of the nuclear genome. If a threshold proportion of mitochondria in a cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved.

### SUMMARY

The present invention encompasses the recognition that administration of folinic acid, glycine or a pharmaceutically acceptable salt thereof, represents an effective therapy for a mitochondrial dysfunction or disorder, autism, and/or Pediatric Acute-onset Neuropsychiatric Syndrome (PANS), wherein the individual has one or more loss-of function mutations in DNA encoding one or more proteins involved or implicated in the folate pathway (e.g., folate metabolism). In some embodiments, one or more folate pathway loss-of-function mutations are in DNA encoding one or more proteins selected from the group consisting of aldehyde dehydrogenase 1 family, member L1 (ALDH1L1), aldehyde dehydrogenase 1 family, member L2 (ALDH1L2), folate receptor 1 (FOLR1), folylpolyglutamate synthase (FPGS), glycine cleavage system H protein (GCSH), glycine cleavage system P protein (GLDC), C-1-tetrahydrofolate synthase (cytoplasmic) (MTHFD1) monofunctional C1-tetrahydrofolate synthase, mitochondrial (MTHFD1L). bifunctional methylenetetrahydrofolate dehydrogenase/cyclohydrolase (MTHFD2), methylenetetrahydrofolate dehydrogenase (NADP-+ dependent) 2-like (MTHFD2L), 5,10-methenyltetrahydrofolate synthetase (MTHFS), methionine synthase reductase (MTRR), serine hydroxymethyltransferase 1 (SHMT1), serine hydroxymethyltransferase 2 (SHMT2) and solute carrier family 25 (mitochondrial folate carrier) (SLC25A32).

In one aspect, the present invention relates to methods and kits for treating and classifying individuals at risk of or suffering from autism, mitochondrial dysfunctions or disorders and/or PANS, and in particular, autism, mitochondrial dysfunctions or disorders and/or PANS dysfunctions or disorders associated with loss of function mutations in genes in the folate pathway, referred to hereafter as “folate metabolism loss-of-function”. In some embodiments dysfunction or disorders associated with folate metabolism loss-of function are treated with folinic acid, glycine or a pharmaceutically acceptable salt thereof.

In certain embodiments, the present invention provides methods of treating an individual at risk of or suffering from a disorder associated with folate metabolism loss-of function (e.g., autism, mitochondrial dysfunctions or disorders and/or PANS), the method comprising administering to the individual a therapeutically effective amount of folinic acid, glycine or a pharmaceutically acceptable salt thereof, wherein nuclear DNA of the individual that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and SLC25A32 includes a loss-of-function mutation.

In certain embodiments, the present invention provides methods of treating an individual at risk of or suffering from a disorder associated with folate metabolism loss-of function (e.g., autism, mitochondrial dysfunctions or disorders and/or PANS), the method comprising administering to the individual a therapeutically effective amount of folinic acid, glycine or a pharmaceutically acceptable salt thereof, wherein, prior to administration, the individual has been determined to possess a loss-of-function mutation in nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and SLC25A32.

In certain embodiments, the present invention provides methods of treating an individual at risk of or suffering from a disorder associated with folate metabolism loss-of function (e.g., autism, mitochondrial dysfunctions or disorders and/or PANS), the method comprising determining that the individual possesses a loss-of-function mutation in nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and SLC25A32 and administering to the individual a therapeutically effective amount of folinic acid, glycine or a pharmaceutically acceptable salt thereof.

In certain embodiments, the present invention provides methods of aiding in the selection of a therapy for an individual at risk of or suffering from a disorder associated with folate metabolism loss-of function (e.g., autism, mitochondrial dysfunctions or disorders and/or PANS), the method comprising obtaining a sample of nuclear DNA from the individual, processing the sample to determine whether the individual possesses a loss-of-function mutation in nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and SLC25A32 and classifying the individual as one that could benefit from therapy with folinic acid, glycine or a pharmaceutically acceptable salt thereof, if the step of processing determines that the individual possesses a loss-of-function mutation in nuclear DNA that encodes ALDH1L1, ALDH1L2, FOLR1, FPGS, GCSH, MTHFD1, MTHFD1L, MTHFD2, MTHFD2L,, MTHFS, MTRR, SHMT1, SHMT2, and/or SLC25A32. In some embodiments, processing comprises sequencing at least a portion of nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32. In some embodiments, the methods further comprise administering to the individual a therapeutically effective amount of folinic acid, glycine or a pharmaceutically acceptable salt thereof.

In certain embodiments, the present invention provides methods of classifying an individual at risk of or suffering from a disorder associated with folate metabolism loss-of function (e.g., autism, mitochondrial dysfunctions or disorders and/or PANS), the method comprising obtaining a sample of nuclear DNA from the individual, processing the sample to determine whether the individual possesses a mutation in nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and SLC25A32, and classifying the individual as one that does or does not possess a mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32. In some embodiments, processing comprises sequencing at least a portion of nuclear DNA that encodes ALDH1L1, ALDH1L2, FOLR1, FPGS, GOSH, GLDC, MTHFD1, MTHFD1L, MTHFD2, MTHFD2L, MTHFS, MTRR, SHMT1, SHMT2, and/or SLC25A32. In some embodiments, the methods further comprise providing the individual or a physician treating the individual with information regarding the mutation. In some embodiments, the information references a correlation between the mutation and the potential benefits of therapy with folinic acid, glycine or a pharmaceutically acceptable salt thereof.

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 23, 64-107, 117, 333, 448, 524, 666, 760, 771 and/or 876 of a ALDH1L1 gene product. In certain embodiments, the present disclosure provides kits for classifying an individual at risk of or suffering from a disorder associated with ALDH1L1 loss-of function, the kit comprising primers for amplifying a target region of nuclear DNA encompassing a region of the ALDH1L1 gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 204, 603, 748, 796, 833 and/or 918 of a ALDH1L2 gene product. In certain embodiments, the present disclosure provides kits for classifying an individual at risk of or suffering from a disorder associated with ALDH1L2 loss-of function, the kit comprising primers for amplifying a target region of nuclear DNA encompassing a region of the ALDH1L2 gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acid 98 of a FOLR1 gene product. In certain embodiments, the present disclosure provides kits for classifying an individual at risk of or suffering from a disorder associated with FOLR1 loss-of function, the kit comprising primers for amplifying a target region of nuclear DNA encompassing a region of the FOLR1 gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 50, 85, 162 and/or 466 of a FPGS gene product. In certain embodiments, the present disclosure provides kits for classifying an individual at risk of or suffering from a disorder associated with FPGS loss-of function, the kit comprising primers for amplifying a target region of nuclear DNA encompassing a region of the FPGS gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acid 84 of a GCSH gene product. In certain embodiments, the present disclosure provides kits for classifying an individual at risk of or suffering from a disorder associated with GCSH loss-of function, the kit comprising primers for amplifying a target region of nuclear DNA encompassing a region of the GCSH gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 18, 147, 503, 675, 705, 716, 895, 937 and/or 966 of a GLDC gene product. In certain embodiments, the present disclosure provides kits for classifying an individual at risk of or suffering from a disorder associated with GLDC loss-of function, the kit comprising primers for amplifying a target region of nuclear DNA encompassing a region of the GLDC gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acid 830 of a MTHFD1 gene product. In certain embodiments, the present disclosure provides kits for classifying an individual at risk of or suffering from a disorder associated with MTHFD1 loss-of function, the kit comprising primers for amplifying a target region of nuclear DNA encompassing a region of the MTHFD1 gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 31, 520, 564 and/or 949 of a MTHFD1L gene product. In certain embodiments, the present disclosure provides kits for classifying an individual at risk of or suffering from a disorder associated with MTHFD1L loss-of function, the kit comprising primers for amplifying a target region of nuclear DNA encompassing a region of the MTHFD1L gene, wherein said region includes one or more sites of loss-of-function mutations that are associated with a disorder associated with folate metabolism loss-of function (e.g., autism, mitochondria' dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acid 263 of a MTHFD2 gene product. In certain embodiments, the present disclosure provides kits for classifying an individual at risk of or suffering from a disorder associated with MTHFD2 loss-of function, the kit comprising primers for amplifying a target region of nuclear DNA encompassing a region of the MTHFD2 gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acid 161 of a MTHFD2L gene product. In certain embodiments, the present disclosure provides kits for classifying an individual at risk of or suffering from a disorder associated with MTHFD2L loss-of function, the kit comprising primers for amplifying a target region of nuclear DNA encompassing a region of the MTHFD2L gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 133 and/or 174 of a MTHFS gene product. In certain embodiments, the present disclosure provides kits for classifying an individual at risk of or suffering from a disorder associated with MTHFS loss-of function, the kit comprising primers for amplifying a target region of nuclear DNA encompassing a region of the MTHFS gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 317 and/or 517 of a MTRR gene product. In certain embodiments, the present disclosure provides kits for classifying an individual at risk of or suffering from a disorder associated with MTRR loss-of function, the kit comprising primers for amplifying a target region of nuclear DNA encompassing a region of the MTRR gene, wherein said region includes one or more sites of loss-of-function mutations that are associated with a disorder associated with folate metabolism loss-of function (e.g., autism, mitochondrial dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 1, 191 and/or 344 of a SHMT1 gene product. In certain embodiments, the present disclosure provides kits for classifying an individual at risk of or suffering from a disorder associated with MTHFS loss-of function, the kit comprising primers for amplifying a target region of nuclear DNA encompassing a region of the SHMT1 gene, wherein said region includes one or more sites of loss-of-function mutations that are associated with a disorder associated with folate metabolism loss-of function (e.g., autism, mitochondria' dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 193 and/or 327 of a SHMT2 gene product. In certain embodiments, the present disclosure provides kits for classifying an individual at risk of or suffering from a disorder associated with SHMT2 loss-of function, the kit comprising primers for amplifying a target region of nuclear DNA encompassing a region of the SHMT2 gene, wherein said region includes one or more sites of loss-of-function mutations that are associated with a disorder associated with folate metabolism loss-of function (e.g., autism, mitochondria' dysfunctions or disorders and/or PANS).

In certain embodiments, the present invention provides kits for classifying an individual at risk of or suffering from a disorder associated with folate metabolism loss-of function (e.g., autism, mitochondrial dysfunctions or disorders and/or PANS), the kit comprising primers for amplifying a target region of nuclear DNA that encompasses part or all of the codon for amino acids 163 and/or 327 of a SLC25A32 gene product. In certain embodiments, the present disclosure provides kits for classifying an individual at risk of or suffering from a disorder associated with SLC25A32 loss-of function, the kit comprising primers for amplifying a target region of nuclear DNA encompassing a region of the SLC25A32 gene, wherein said region includes one or more sites of loss-of-function mutations that are associated with a disorder associated with folate metabolism loss-of function (e.g., autism, mitochondria' dysfunctions or disorders and/or PANS).

In some embodiments, according to the methods and kits described herein, the disorder associated with folate metabolism loss-of function (e.g., autism, mitochondrial dysfunctions or disorders and/or PANS) is selected from the group consisting of abnormal autonomic activity, functional gastrointestinal disorders, chronic pain disorders, autistic spectrum disorders, psychiatric disorders, cognitive dysfunction, and combinations thereof In some embodiments, the individual has suffered with any combination of chronic signs, symptoms, conditions, or diagnoses that include pain, fatigue, and/or digestive system dysfunction prior to administration. In some embodiments, the individual has suffered from episodic dementia/psychosis prior to administration. In some embodiments, the individual has suffered from intestinal pseudo-obstruction prior to administration. In some embodiments, the individual has suffered from an autistic spectrum disorder prior to administration. In some embodiments, the individual has suffered from PANS prior to administration. In some embodiments, the individual has suffered from intermittent encephalopathy prior to administration. In some embodiments, the individual has suffered from dementia prior to administration. In some embodiments, the individual has suffered from cognitive decline prior to administration. In some embodiments, the individual has suffered from migraines prior to administration. In some embodiments, the individual has suffered an adverse reaction to an anticholinergic medication prior to administration.

In some embodiments, according to the methods and kits described herein, the individual suffers from a mitochondrial dysfunction. In some embodiments, the individual further possesses homoplasmic mitochondrial DNA variants. In some embodiments, the methods described herein further comprise sequencing mitochondrial DNA obtained from the individual. In some embodiments, the mitochondrial DNA of the individual has been sequenced without identifying heteroplasmic mitochondrial DNA variants.

In some embodiments, according to the methods and kits described herein, the loss-of-function mutation causes reduced expression of a ALDH1L1 gene product. In some embodiments, the loss-of-function mutation is in the regulatory sequence of the ALDH1L1 gene. In some embodiments, the loss-of-function mutation is in the coding sequence of the ALDH1L1 gene. In some embodiments, the loss-of-function mutation causes reduced activity of a ALDH1L1 gene product. In some embodiments, the loss-of-function mutation is or comprises a mutation selected from the group consisting of 23G>D, 117S>L, 333R>Q, 448S>N, 524G>S, 666N>K, 760E>K771T>A, 876K>R, frame shift p.Ala107Profs64X, and combinations thereof.

In some embodiments, according to the methods and kits described herein, the loss-of-function mutation causes reduced expression of a ALDH1L2 gene product. In some embodiments, the loss-of-function mutation is in the regulatory sequence of the ALDH1L2 gene. In some embodiments, the loss-of-function mutation is in the coding sequence of the ALDH1L2 gene. In some embodiments, the loss-of-function mutation causes reduced activity of a ALDH1L2 gene product. In some embodiments, the loss-of-function mutation is or comprises a mutation selected from the group consisting of 204L>F, 603W>X, 748V>A, 796G>R, 833T>I, 918T>M, and combinations thereof.

In some embodiments, according to the methods and kits described herein, the loss-of-function mutation causes reduced expression of a FOLR1 gene product. In some embodiments, the loss-of-function mutation is in the regulatory sequence of the FOLR1 gene. In some embodiments, the loss-of-function mutation is in the coding sequence of the FOLR1 gene. In some embodiments, the loss-of-function mutation causes reduced activity of a FOLR1 gene product. In some embodiments, the loss-of-function mutation is or comprises a mutation consisting of 98R>W.

In some embodiments, according to the methods and kits described herein, the loss-of-function mutation causes reduced expression of a FPGS gene product. In some embodiments, the loss-of-function mutation is in the regulatory sequence of the FPGS gene. In some embodiments, the loss-of-function mutation is in the coding sequence of the FPGS gene. In some embodiments, the loss-of-function mutation causes reduced activity of a FPGS gene product. In some embodiments, the loss-of-function mutation is or comprises a mutation selected from the group consisting of 50R>C, 85R>W, 162R>Q, 466R>C, and combinations thereof.

In some embodiments, according to the methods and kits described herein, the loss-of-function mutation causes reduced expression of a GCSH gene product. In some embodiments, the loss-of-function mutation is in the regulatory sequence of the GCSH gene. In some embodiments, the loss-of-function mutation is in the coding sequence of the GCSH gene. In some embodiments, the loss-of-function mutation causes reduced activity of a GCSH gene product. In some embodiments, the loss-of-function mutation is or comprises a mutation consisting of 84Y>H.

In some embodiments, according to the methods and kits described herein, the loss-of-function mutation causes reduced expression of a GLDC gene product. In some embodiments, the loss-of-function mutation is in the regulatory sequence of the GLDC gene. In some embodiments, the loss-of-function mutation is in the coding sequence of the GLDC gene. In some embodiments, the loss-of-function mutation causes reduced activity of a GLDC gene product. In some embodiments, the loss-of-function mutation is or comprises a mutation selected from the group consisting of 18G>C, 147I>M, 503E>A, 675N>K, 705V>M, 716L>H, 895M>V, 937R>L, 966Q>H, and combinations thereof.

In some embodiments, according to the methods and kits described herein, the loss-of-function mutation causes reduced expression of a MTHFD1 gene product. In some embodiments, the loss-of-function mutation is in the regulatory sequence of the MTHFD1 gene. In some embodiments, the loss-of-function mutation is in the coding sequence of the MTHFD1 gene. In some embodiments, the loss-of-function mutation causes reduced activity of a MTHFD1 gene product. In some embodiments, the loss-of-function mutation is or comprises a mutation consisting of 830A>V.

In some embodiments, according to the methods and kits described herein, the loss-of-function mutation causes reduced expression of a MTHFD1L gene product. In some embodiments, the loss-of-function mutation is in the regulatory sequence of the MTHFD1L gene. In some embodiments, the loss-of-function mutation is in the coding sequence of the MTHFD1L gene. In some embodiments, the loss-of-function mutation causes reduced activity of a MTHFD1L gene product. In some embodiments, the loss-of-function mutation is or comprises a mutation selected from the group consisting of 31A>G, 520Y>C, 564R>H, 949G>R, and combinations thereof.

In some embodiments, according to the methods and kits described herein, the loss-of-function mutation causes reduced expression of a MTHFD2 gene product. In some embodiments, the loss-of-function mutation is in the regulatory sequence of the MTHFD2 gene. In some embodiments, the loss-of-function mutation is in the coding sequence of the MTHFD2 gene. In some embodiments, the loss-of-function mutation causes reduced activity of a MTHFD2 gene product. In some embodiments, the loss-of-function mutation is or comprises a mutation consisting of 263D>G.

In some embodiments, according to the methods and kits described herein, the loss-of-function mutation causes reduced expression of a MTHFD2L gene product. In some embodiments, the loss-of-function mutation is in the regulatory sequence of the MTHFD2L gene. In some embodiments, the loss-of-function mutation is in the coding sequence of the MTHFD2L gene. In some embodiments, the loss-of-function mutation causes reduced activity of a MTHFD2L gene product. In some embodiments, the loss-of-function mutation is or comprises a mutation selected from the group consisting of 161G>E, 210V>L, and combinations thereof.

In some embodiments, according to the methods and kits described herein, the loss-of-function mutation causes reduced expression of a MTHFS gene product. In some embodiments, the loss-of-function mutation is in the regulatory sequence of the MTHFS gene. In some embodiments, the loss-of-function mutation is in the coding sequence of the MTHFS gene. In some embodiments, the loss-of-function mutation causes reduced activity of a MTHFS gene product. In some embodiments, the loss-of-function mutation is or comprises a mutation selected from the group consisting of 133L>Q, 174E>K, and combinations thereof.

In some embodiments, according to the methods and kits described herein, the loss-of-function mutation causes reduced expression of a MTRR gene product. In some embodiments, the loss-of-function mutation is in the regulatory sequence of the MTRR gene. In some embodiments, the loss-of-function mutation is in the coding sequence of the MTRR gene. In some embodiments, the loss-of-function mutation causes reduced activity of a MTRR gene product. In some embodiments, the loss-of-function mutation is or comprises a mutation selected from the group consisting of 317I>T, 517T>A, and combinations thereof.

In some embodiments, according to the methods and kits described herein, the loss-of-function mutation causes reduced expression of a SHMT1 gene product. In some embodiments, the loss-of-function mutation is in the regulatory sequence of the SHMT1 gene. In some embodiments, the loss-of-function mutation is in the coding sequence of the SHMT1 gene. In some embodiments, the loss-of-function mutation causes reduced activity of a SHMT1 gene product. In some embodiments, the loss-of-function mutation is or comprises a mutation selected from the group consisting of 1M>R, 1M>K, 191R>C, 344E>Q, and combinations thereof.

In some embodiments, according to the methods and kits described herein, the loss-of-function mutation causes reduced expression of a SHMT2 gene product. In some embodiments, the loss-of-function mutation is in the regulatory sequence of the SHMT2 gene. In some embodiments, the loss-of-function mutation is in the coding sequence of the SHMT2 gene. In some embodiments, the loss-of-function mutation causes reduced activity of a SHMT2 gene product. In some embodiments, the loss-of-function mutation is or comprises a mutation selected from the group consisting of 193R>Q, 327R>Q, and combinations thereof.

In some embodiments, according to the methods and kits described herein, the loss-of-function mutation causes reduced expression of a SLC25A32 gene product. In some embodiments, the loss-of-function mutation is in the regulatory sequence of the SLC25A32 gene. In some embodiments, the loss-of-function mutation is in the coding sequence of the SLC25A32 gene. In some embodiments, the loss-of-function mutation causes reduced activity of a SLC25A32 gene product. In some embodiments, the loss-of-function mutation is or comprises a mutation selected from the group consisting of 163Y>C, 300Y>C, and combinations thereof.

In some embodiments, according to the methods and kits described herein, the loss-of-function mutation is heterozygous.

In some embodiments, according to the methods and kits described herein, the loss-of-function mutation is homozygous.

In some embodiments, according to the methods and kits described herein, the loss-of-function mutation is a frame shift mutation.

The present invention also provides, among other things, a method of building a database for use in selecting a medication (e.g., folinic acid, glycine or a pharmaceutically acceptable salt thereof) for an individual. The method includes receiving, in a computer system, a plurality of genotyped polymorphisms for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and/or SLC25A32; receiving a plurality of medication profiles specified based on the polymorphisms; and storing the plurality of polymorphisms and the medication profiles such that each medication profile is associated with one of the genotypes. The at least one medication profile can identify a medication and the medication can be placed in one of multiple categories included in the medication profile. Such categories can be selected from the group consisting of: medications that are safe to use, medications that should be used with caution, medications that should be closely monitored when used, medications that should be avoided, and combinations thereof The medication profile can identify a universe of possible medications for the individual's genotype.

In another aspect, the invention features a computer program product containing executable instructions that when executed cause a processor to perform operations. The operations can include: receive a plurality of genotyped polymorphisms for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and/or SLC25A32; receive a plurality of medication profiles specified based on the genotypes; and store the genotypes and the medication profiles such that each medication profile is associated with one of the genotypes.

The invention also features a method of selecting a medication (e.g., folinic acid, glycine or a pharmaceutically acceptable salt thereof) for an individual. The method includes receiving, in a computer system, an individual's genotyped polymorphisms for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and/or SLC25A32; identifying, in a database comprising a plurality of medication profiles associated with genotypes, a medication profile that is associated with the individual's genotype; and outputting the identified medication profile in response to receiving the individual's genotype. A user can enter the individual's genotype in the computer system or the individual's genotype can be received directly from equipment used in determining the individual's genotype.

The medication profile can include a ranking of several medications, e.g., based on specific co-factors (e.g., clinical symptoms). The method can include adjusting the ranking before outputting the identified medication profile (e.g., based on receiving a genotypic polymorphism carried by the individual or based on receiving a clinical response relating to the individual). The clinical response can be by a family member of the individual.

In yet another aspect, the invention features a computer program product containing executable instructions that when executed cause a processor to perform operations that include receive an individual's genotyped polymorphisms for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and/or SLC25A32; identify, in a database including a plurality of medication profiles associated with genotypes, a medication profile that is associated with the individual's genotype; and output the identified medication profile in response to receiving the individual's genotype.

### BRIEF DESCRIPTION OF THE DRAWING

The Figures described below, that together make up the Drawing, are for illustration purposes only, not for limitation.

FIG. 1: depicts an exemplary block diagram of a computer system 100.

FIG. 2: depicts an exemplary flow chart of a method 200 for building a database for use in selecting a medication for an individual.

FIG. 3: depicts an exemplary flow chart of a method 300 for selecting medication for an individual.

### DEFINITIONS

Associated With: The term “associated with” is used herein to describe an observed correlation between two items or events. For example, a loss-of-function mutation in the folate pathway may be considered to be “associated with” a particular neurological and/or mitochondrial dysfunction or disorder (e.g., autism, mitochondrial dysfunctions or disorders and/or PANS) if its presence or level correlates with a presence or level of the dysfunction or disorder.

Coding sequence: As used herein, the term “coding sequence” refers to a sequence of a nucleic acid or its complement, or a part thereof, that can be transcribed and/or translated to produce the mRNA for and/or the polypeptide or a fragment thereof. Coding sequences include exons in genomic DNA or immature primary RNA transcripts, which are joined together by the cell's biochemical machinery to provide a mature mRNA.

Dosage form: As used herein, the terms “dosage form” and “unit dosage form” refer to a physically discrete unit of a therapeutic composition for administration to a subject to be treated. Each unit dosage form contains a predetermined quantity of active agent (for example, folinic acid, glycine or a pharmaceutically acceptable salt thereof) calculated to produce a desired therapeutic effect when administered in accordance with a dosing regimen. It will be understood, however, that a total dosage of the active agent may be decided by an attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent (for example, folinic acid, glycine or a pharmaceutically acceptable salt thereof) has a recommended dosing regimen, which may involve one or more doses.

Gene: The term “gene”, as used herein, has its art understood meaning, and refers to a part of the genome specifying a macromolecular product, be it DNA for incorporation into a host genome, a functional RNA molecule or a protein, and may include regulatory sequences (e.g., promoters, enhancers, etc.) and/or intron sequences preceding (5′ non-coding sequences

Heteroplasmic mitochondrial DNA variants: As used herein, the term “heteroplasmic mitochondrial DNA variants” refers to a mutation in mitochondrial DNA that affects a proportion of the mitochondrial DNA, while the remaining mitochondrial DNA is wild-type. In some embodiments, 0, 1, 2, 3, 4, 5, 6, 7, 8, 9, 10% or more of the mitochondrial DNA possesses the mutation.

Homoplasmic mitochondrial DNA variants: As used herein, the term “homoplasmic mitochondrial DNA variants” refers to a mutation in mitochondrial DNA that affects substantially all of the mitochondrial DNA

Loss-of-function mutation: As used herein, the term “loss-of-function mutation” refers to a mutation that is associated with a reduction or elimination of the normal activity of a gene or gene product. Loss of activity can be due to a decrease in transcription and/or processing of the RNA, a decrease in translation, stability, transport, or activity of the gene product, or any combination thereof. In some embodiments, normal activity of a gene or gene product is reduced from 0, 1, 2, 3, 4, 5, 6, 7, 8, 9, 10 up to 100%.

Mitochondrial DNA: As used herein, the term “mitochondrial DNA” refers to the part of the genome that is located in the mitochondria of a cell.

Mutation: As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the reduction or elimination of an existing character, property, function, phenotype or trait not found in the protein encoded by the parental sequence.

Nuclear DNA: As used herein, the term “nuclear DNA” refers to the part of the genome that is located in the nucleus of a cell.

Nucleic Acid: The terms “nucleic acid”, “nucleic acid molecule”, and “polynucleotide” each is used herein to refer to a polymers of nucleotide monomers or analogs thereof, such as deoxyribonucleic acid (DNA) and ribonucleic acid (RNA). Unless otherwise stated, the terms encompass nucleic acid-like structures with synthetic backbones, as well as amplification products. In some embodiments, nucleic acids involved in the present invention are linear nucleic acids.

Primer: The terms “primer”, as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least about 8, e.g., at least about 10, at least about 15, or about 20 to about 40 consecutive nucleotides of a target nucleic acid (i.e., will hybridize to a contiguous sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

Reference: As will be understood from context, a reference sequence, sample, population, agent or individual is one that is sufficiently similar to a particular sequence, sample, population, agent or individual of interest to permit a relevant comparison (i.e., to be comparable). In some embodiments, information about a reference sample is obtained simultaneously with information about a particular sample. In some embodiments, information about a reference sample is historical. In some embodiments, information about a reference sample is stored for example in a computer-readable medium. In some embodiments, comparison of a particular sample of interest with a reference sample establishes identity with, similarity to, or difference of a particular sample of interest relative to a reference.

Regulatory Sequence: The term “regulatory sequence” is intended to include promoters, enhancers and other expression control elements (e.g., polyadenylation signals).

Risk: As will be understood from context, a “risk” of a disease, disorder or condition (e.g., a neurological dysfunction or disorder) comprises a likelihood that a particular individual will develop the disease, disorder, or condition. In some embodiments, risk is expressed as a percentage. In some embodiments, risk is from 0, 1, 2, 3, 4, 5, 6, 7, 8, 9, 10 up to 100%. In some embodiments risk is expressed as a risk relative to a risk associated with a reference sample or group of reference samples. In some embodiments, a reference sample or group of reference samples have a known risk of a disease, disorder, or condition (e.g., a mitochondrial and/or neurological dysfunction or disorder). In some embodiments a reference sample or group of reference samples are from individuals comparable to a particular individual. In some embodiments, relative risk is 0, 1, 2, 3, 4, 5, 6, 7, 8, 9, 10 or more.

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids extracted from a sample or obtained by subjecting a primary sample to techniques such as amplification, isolation and/or purification of certain components, etc.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” refers to an amount of a therapeutic composition (e.g., folinic acid, glycine which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, a “therapeutically effective amount” refers to an amount of a therapeutic composition effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease. A therapeutically effective amount is commonly administered in a dosing regimen that may comprise multiple unit doses. A therapeutically effective amount (and/or an appropriate unit dose within an effective dosing regimen) may vary, for example, depending on route of administration, combination with other agents, etc.

Treatment: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Wild type: As used herein, the term “wild-type” refers to a typical or common form existing in nature; in some embodiments it is the most common form.

### Detailed Description of Certain Embodiments

Folate Metabolism and Biological Roles

Folic acid (also known as folate, vitamin M, vitamin B₉, vitamin B_{c} (or folacin), pteroyl-L-glutamic acid, pteroyl-L-glutamate, and pteroylmonoglutamic acid) are forms of water-soluble vitamin B₉. Folate is composed of the aromatic pteridine ring linked to para-aminobenzoic acid and one or more glutamate residues. Folic acid is itself not biologically active and requires metabolic processing via the folate pathway into one of several biological active derivatives (e.g., biologically active tetrahydrofolate is converted from dihydrofolic acid in the liver).

Vitamin B₉ (e.g., folic acid and folate) is essential for numerous bodily functions including DNA synthesis, DNA repair and DNA methylation and also acts as a cofactor in certain biological reactions. Folate also plays a role in aiding rapid cell division and growth, such as during infancy and pregnancy. Humans cannot synthesize folate de novo; therefore, folate has to be supplied through the diet to meet their daily requirements. Children and adults both require folic acid to produce healthy red blood cells and prevent anemia.

Folate deficiency results in many health problems, the most notable one being neural tube defects in developing embryos. Disruption (e.g., loss-of-function) of proteins involved in folate metabolism (e.g., enzymes in the folate pathway, co-factors, etc.) may lead to folate deficiency due to an inability to convert folates into biologically active derivatives such as tetrahydrofolate.

In some embodiments, symptoms of folate deficiency include diarrhea, macrocytic anemia with weakness or shortness of breath, nerve damage with weakness and limb numbness (peripheral neuropathy), pregnancy complications, mental confusion, forgetfulness or other cognitive declines, mental depression, sore or swollen tongue, peptic or mouth ulcers, headaches, heart palpitations, irritability, and behavioral disorders. Low levels of folate can also lead to homocysteine accumulation. DNA synthesis and repair are impaired and this could lead to cancer development.

ALDH1L1 and ALDH1L2

Aldehyde dehydrogenase 1 family, member L1 (ALDH1L1) and aldehyde dehydrogenase 1 family, member L2 (ALDH1L2) (sometimes referred to as mitochondrial 10-formyltetrahydrofolate dehydrogenase precursor) are enzymes that catalyzes the conversion of 10-formyltetrahydrofolate, nicotinamide adenine dinucleotide phosphate (NADP+), and water to tetrahydrofolate, NADPH, and carbon dioxide. ALDH1L1 and ALDH1L2 have been purified, characterized, cloned and sequenced from human sources. Human ALDH1L1 protein (NP_001257293.1; SEQ ID NO: 1) contains 912 amino acid residues. Human ALDH1L2 protein (NP_001029345.2; SEQ ID NO: 3) contains 923 amino acid residues. Exemplary amino acid and nucleotide sequence from a full-length human ALDH1L1 polypeptide are shown below in Table 1 as SEQ ID NOs: 1 and 2. Exemplary amino acid and nucleotide sequence from a full-length human ALDH1L2 polypeptide are shown below in Table 1 as SEQ ID NOs: 3 and 4.

FOLR1

Folate receptor alpha (FOLR1) is a member of the folate receptor family and has a high affinity for folic acid and for several reduced folic acid derivatives and mediate delivery of 5-methyltetrahydrofolate to the interior of cells. FOLR1 has been purified, characterized, cloned and sequenced from human sources. Human FOLR1 has four variants, all of which encode the same protein; FOLR1 variant (7) represents the longest variant (NP_057936.1; SEQ ID NO: 5) and contains 257 amino acid residues. Exemplary amino acid and nucleotide sequence from a full-length human FOLR1 polypeptide are shown below in Table 1 as SEQ ID NOs: 5 and 6.

FPGS

Folylpolyglutamate synthase, mitochondrial (FPGS) is a folylpolyglutamate synthetase enzyme that is involved in establishing and maintaining both cytosolic and mitochondrial folylpolyglutamate concentrations and plays a role in folate homeostasis and survival of proliferating cells. FPGS catalyzes ATP-dependent addition of glutamate moieties to folate and folate derivatives. FPGS variant (1) represents the longer transcript and encodes isoform (a). Human FPGS variant (1) has two alternative translational start codons in the same reading frame which encode either a longer, signal-containing mitochondrial protein (NP_004948.4; SEQ ID NO: 7) which contains 587 amino acid residues or a shorter, signal-less cytosolic protein. Exemplary amino acid and nucleotide sequence from a full-length human FPGS polypeptide are shown below in Table 1 as SEQ ID NOs: 7 and 8.

GCSH and GLDC

Glycine cleavage system H protein, mitochondrial (GCSH) is part of a 4 component glycine cleavage system (P protein, H protein, T protein, and L protein) which is confined to the mitochondria. GCSH shuttles the methylamine group of glycine from the P protein to the T protein. Human GCSH (NP_004474.2; SEQ ID NO: 9) contains 173 amino acid residues. Exemplary amino acid and nucleotide sequence from a full-length human GCSH polypeptide are shown below in Table 1 as SEQ ID NOs: 9 and 10.

Glycine cleavage system P protein (GLDC) is a pyridoxal phosphate-dependent glycine decarboxylase which binds the alpha-amino group of glycine through its pyridoxal phosphate cofactor. Carbon dioxide is released and the remaining methylamine moiety is then transferred to the lipoamide cofactor of the H protein. Human GLDC (NP_000161.2; SEQ ID NO: 11) contains 1020 amino acid residues. Exemplary amino acid and nucleotide sequence from a full-length human GLDC polypeptide are shown below in Table 1 as SEQ ID NOs: 11 and 12.

MTHFD1

C-1-tetrahydrofolate synthase, cytoplasmic (also known as C1-THF synthase, methylenetetrahydrofolate dehydrogenase (NADP+ dependent) 1, methenyltetrahydrofolate cyclohydrolase, formyltetrahydrofolate synthetase) (MTHFD1) is a trifunctional enzyme with three distinct enzymatic activities: methylenetetrahydrofolate dehydrogenase, methenyltetrahydrofolate cyclohydrolase and formate-tetrahydrofolate ligase. Each of these activities catalyzes one of three sequential reactions in the interconversion of 1-carbon derivatives of tetrahydrofolate, which are substrates for methionine, thymidylate, and de novo purine syntheses. The trifunctional enzymatic activities are conferred by two major domains, an amino terminal portion containing the dehydrogenase and cyclohydrolase activities and a larger synthetase domain. Human MTHFD1 (NP_005947.3; SEQ ID NO: 13) contains 935 amino acid residues. Exemplary amino acid and nucleotide sequence from a full-length human MTHFD1 polypeptide are shown below in Table 1 as SEQ ID NOs: 13 and 14.

MTHFD1L

Monofunctional C1-tetrahydrofolate synthase, mitochondrial (also known as formyltetrahydrofolate synthetase)(MTHFD1L)is involved in the synthesis of tetrahydrofolate in the mitochondrion. Human MTHFD1L (NP_001229696.1; SEQ ID NO: 15) contains 797 amino acid residues. Exemplary amino acid and nucleotide sequence from a full-length human MTHFD1L polypeptide are shown below in Table 1 as SEQ ID NOs: 15 and 16.

MTHFD2

Bifunctional methylenetetrahydrofolate dehydrogenase/cyclohydrolase, mitochondrial (MTHFD2) is a nuclear-encoded mitochondrial bifunctional enzyme with methylenetetrahydrofolate dehydrogenase and methenyltetrahydrofolate cyclohydrolase activities. Human MTHFD2 (NP_006627.2; SEQ ID NO: 17) contains 350 amino acid residues. Exemplary amino acid and nucleotide sequence from a full-length human MTHFD2 polypeptide are shown below in Table 1 as SEQ ID NOs: 17 and 18.

MTHFD2L

Methylenetetrahydrofolate dehydrogenase (NADP+ dependent) 2-like (MTHFD2L) is a probable bifunctional methylenetetrahydrofolate dehydrogenase/cyclohydrolase. Human MTHFD2L (NP_001138450.1; SEQ ID NO: 19) contains 347 amino acid residues. Exemplary amino acid and nucleotide sequence from a full-length human MTHFD2L polypeptide are shown below in Table 1 as SEQ ID NOs: 19 and 20.

MTHFS

5,10-methenyltetrahydrofolate synthetase (5-formyltetrahydrofolate cyclo-ligase) (MTHFS) is an enzyme that catalyzes the conversion of 5-formyltetrahydrofolate to 5,10-methenyltetrahydrofolate, a precursor of reduced folates involved in 1-carbon metabolism. Increased activity of MTHFS can result in an increased folate turnover rate and folate depletion. Human MTHFS (NP_006432.1; SEQ ID NO: 21) contains 203 amino acid residues. Exemplary amino acid and nucleotide sequence from a full-length human MTHFS polypeptide are shown below in Table 1 as SEQ ID NOs: 21 and 22.

MTRR

Methionine synthase reductase, mitochondrial (MTRR) regenerates a functional methionine synthase via reductive methylation (methionine synthase eventually becomes inactive due to the oxidation of its cob(I)alamin cofactor). Human MTRR (NP_002445.2; SEQ ID NO: 23) contains 698 amino acid residues. Exemplary amino acid and nucleotide sequence from a full-length human MTRR polypeptide are shown below in Table 1 as SEQ ID NOs: 23 and 24.

SHMT1 and SHMT2

Serine hydroxymethyltransferase (SHMT) a pyridoxal phosphate-containing enzyme which is primarily responsible for glycine synthesis and is a primary source for intracellular glycine. SHMT plays an important role in cellular one-carbon pathways by catalyzing the reversible, simultaneous conversions of L-serine to glycine (retro-aldol cleavage) and tetrahydrofolate to 5,10-methylenetetrahydrofolate (hydrolysis). This reaction provides the largest part of the one-carbon units available to the cell. Decreased SHMT (and/or SHMT activity) results in less available glycine which affects the nervous system by acting as an agonist to the NMDA receptor. Mammals have cytoplasmic (soluable) and mitochondrial isoforms. SHMT1 encodes the soluable cytoplasmic form of the enzyme. SHMT2 encodes the mitochondrial form of the enzyme. Human SHMT1 (NP_004160.3; SEQ ID NO: 25) contains 483 amino acid residues. Human SHMT2 (NP 005403.2; SEQ ID NO: 27) contains 504 amino acid residues. Exemplary amino acid and nucleotide sequence from full-length human SHMT1 and SHMT2 polypeptides are shown below in Table 1 as SEQ ID NOs: 25, 26, 27 and 28.

SLC25A32

Solute carrier family 25 (mitochondrial folate carrier), member 32 (SLC25A32) is a member of the P(I/L)W subfamily of mitochondrial carrier family transport proteins. SLC25A32 transports folate across the inner mitochondrial membrane. Human SLC25A32 (NP_110407.2; SEQ ID NO: 29) contains 315 amino acid residues. Exemplary amino acid and nucleotide sequence from a full-length human SLC25A32polypeptide are shown below in Table 1 as SEQ ID NOs: 29 and 30.

TABLE 1
Exemplary Folate Pathway sequences

| Human ALDH1L1 | MAGPSNPPATMKIAVIGQSLFGQEVYCHLRKEGHEVVGVFTVPDK |
|---|---|
| Protein Sequence | DGKADPLGLEAEKDGVPVFKYSRWRAKGQALPDVVAKYQALGAEL |
| cytosolic 10- | NVLPFCSQFIPMEIISAPRHGSIIYHPSLLPRHRGASAINWTLIH |
| formyltetrahydro- | GDKKGGFSIFWADDGLDTGDLLLQKECEVLPDDTVSTLYNRFLFP |
| folate dehydrogenase | EGIKGMVQAVRLIAEGKAPRLPQPEEGATYEGIQKKETAKINWDQ |
| isoform 1 | PAEAIHNWIRGNDKVPGAWTEACEQKLTFFNSTLNTSGLVPEGDA |
| (NCBI Reference | LPIPGAHRPGVVTKAGLILFGNDDKMLLVKNIQLEDGKMILASNF |
| Sequence: | FKGAASSVLELTEAELVTAEAVRSVWQRILPKVLEVEDSTDFFKS |
| NP_001257293.1) | GAASVDVVRLVEEVKELCDGLELENEDVYMASTFGDFIQLLVRKL |
| | RGDDEEGECSIDYVEMAVNKRTVRMPHQLFIGGEFVDAEGAKTSE |
| | TINPTDGSVICQVSLAQVTDVDKAVAAAKDAFENGRWGKISARDR |
| | GRLMYRLADLMEQHQEELATIEALDAGAVYTLALKTHVGMSIQTF |
| | RYFAGWCDKIQGSTIPINQARPNRNLTLTRKEPVGVCGIIIPWNY |
| | PLMMLSWKTAACLAAGNTVVIKPAQVTPLTALKFAELTLKAGIPK |
| | GVVNVLPGSGSLVGQRLSDHPDVRKIGFTGSTEVGKHIMKSCAIS |
| | NVKKVSLELGGKSPLIIFADCDLNKAVQMGMSSVFFNKGENCIAA |
| | GRLFVEDSIHDEFVRRVVEEVRKMKVGNPLDRDTDHGPQNHHAHL |
| | VKLMEYCQHGVKEGATLVCGGNQVPRPGFFFEPTVFTDVEDHMFI |
| | AKEESFGPVMIISRFADGDLDAVLSRANATEFGLASGVFTRDINK |
| | ALYVSDKLQAGTVFVNTYNKTDVAAPFGGFKQSGFGKDLGEAALN |
| | EYLRVKTVTFEY (SEQ ID NO: 1) |
| Human ALDH1L1 | TCTGCGGCACCAGGACTGAGTAGAAGGGAGAGAGTGGAGAAGGGG |
| mRNA Sequence | AATTGCAGAGAGAAAACCAGGGGCTGTTTTTCTCTCGGAGAGGCG |
| cytosolic 10- | GGTAGGCACTGGGCGGGCAGAAGCGCCGCTATCCACCCGGATGCG |
| formyltetrahydro- | CAGCTGCTAAGGGGCCGCCTCTGCAAGCGGCTGCAAATTCCCGGA |
| folate dehydrogenase | GGGCAGCGTCTCCTTTCGCTCTGCTGTGTCCGTAGCACATGGCAG |
| isoform 1 | GTCCTTCCAACCCTCCTGCTACCATGAAGATTGCAGTGATTGGAC |
| (NCBI Reference | AGAGCCTGTTTGGCCAGGAAGTTTACTGCCACCTGAGGAAGGAGG |
| Sequence: | GCCACGAAGTGGTGGGTGTGTTCACTGTTCCAGACAAGGATGGAA |
| NM_001270364.1) | AGGCCGACCCCCTGGGTCTGGAAGCTGAGAAGGATGGAGTGCCGG |
| | TATTCAAGTACTCCCGGTGGCGTGCAAAAGGACAGGCTTTGCCTG |
| | ATGTGGTGGCAAAATACCAGGCTTTGGGGGCCGAGCTCAACGTCC |
| | TGCCCTTCTGCAGCCAATTCATCCCCATGGAGATAATCAGTGCCC |
| | CCCGGCATGGCTCCATCATCTATCACCCGTCACTGCTCCCTAGGC |
| | ACCGAGGGGCCTCGGCCATCAACTGGACCCTCATTCACGGAGATA |
| | AGAAAGGGGGGTTTTCCATCTTCTGGGCGGATGATGGTCTGGACA |
| | CCGGAGACCTGCTGCTGCAGAAGGAGTGTGAGGTGCTCCCGGACG |
| | ACACCGTGAGCACGCTGTACAACCGCTTCCTCTTCCCTGAAGGCA |
| | TCAAAGGGATGGTGCAGGCCGTGAGGCTGATCGCTGAGGGCAAAG |
| | CCCCCAGACTCCCTCAGCCTGAGGAAGGAGCCACCTATGAGGGGA |
| | TTCAGAAGAAGGAGACAGCCAAGATCAACTGGGACCAGCCGGCAG |
| | AGGCCATTCACAACTGGATCCGCGGGAACGACAAGGTGCCGGGAG |
| | CCTGGACAGAGGCCTGTGAACAGAAACTGACATTTTTCAACTCAA |
| | CGCTGAACACTTCAGGCCTGGTGCCCGAGGGAGACGCTTTGCCCA |
| | TCCCAGGAGCCCATCGGCCAGGGGTGGTCACCAAAGCAGGACTCA |
| | TCCTCTTTGGGAATGATGACAAAATGCTGCTGGTGAAGAATATTC |
| | AGCTGGAGGATGGCAAAATGATCCTGGCCTCGAACTTCTTTAAGG |
| | GGGCAGCCAGCAGTGTCCTTGAGCTGACAGAGGCAGAGCTGGTTA |
| | CTGCGGAGGCTGTGCGGAGTGTTTGGCAGCGGATCCTCCCCAAAG |
| | TCCTGGAGGTTGAAGACTCCACTGATTTCTTCAAGTCAGGGGCCG |
| | CGTCTGTGGACGTTGTGAGGCTGGTGGAGGAAGTGAAGGAGCTGT |
| | GTGATGGCCTGGAGTTAGAAAATGAAGATGTGTACATGGCATCCA |
| | CCTTTGGGGACTTCATCCAGCTGTTAGTGAGGAAGCTGCGAGGGG |
| | ACGATGAGGAGGGCGAGTGCAGCATTGACTACGTGGAAATGGCAG |
| | TGAACAAGCGCACTGTCCGCATGCCCCACCAGCTCTTCATTGGGG |
| | GGGAGTTCGTGGATGCCGAGGGCGCCAAGACCTCTGAGACCATCA |
| | ATCCCACCGATGGAAGTGTCATCTGCCAGGTATCCCTGGCCCAAG |
| | TCACCGACGTCGACAAGGCAGTGGCCGCAGCCAAGGATGCCTTTG |
| | AGAATGGACGGTGGGGGAAGATCAGTGCGCGGGACCGGGGCCGGC |
| | TGATGTACAGGTTGGCAGATCTCATGGAGCAGCACCAGGAGGAGC |
| | TGGCCACCATTGAGGCCCTGGATGCGGGTGCCGTCTACACGCTGG |
| | CCCTGAAGACCCACGTGGGCATGTCCATCCAGACCTTCCGCTACT |
| | TTGCTGGCTGGTGTGACAAGATCCAGGGCTCCACCATCCCCATCA |
| | ACCAGGCCAGACCCAACCGCAACCTGACCTTGACCAGGAAGGAGC |
| | CTGTTGGGGTTTGTGGCATCATCATCCCCTGGAACTATCCCCTGA |
| | TGATGCTGTCCTGGAAGACAGCTGCCTGCCTGGCTGCCGGGAACA |
| | CAGTGGTGATCAAGCCTGCTCAGGTGACCCCACTCACAGCCTTGA |
| | AGTTTGCAGAGCTGACATTAAAGGCCGGCATTCCCAAAGGTGTGG |
| | TTAACGTCCTCCCAGGATCTGGCTCCCTGGTCGGCCAGAGACTCT |
| | CAGACCATCCTGATGTGAGGAAAATCGGGTTCACAGGCTCCACAG |
| | AGGTGGGCAAGCACATCATGAAAAGCTGTGCCATAAGTAACGTGA |
| | AGAAGGTGTCCCTGGAACTGGGCGGGAAGTCACCCCTCATCATCT |
| | TTGCTGACTGTGACCTCAACAAGGCTGTGCAGATGGGGATGAGTT |
| | CTGTTTTCTTCAACAAAGGAGAGAATTGCATTGCAGCAGGCCGAC |
| | TCTTTGTGGAGGACTCCATTCATGATGAGTTCGTGCGGAGAGTGG |
| | TAGAAGAGGTGCGGAAGATGAAGGTGGGCAACCCGCTGGACAGGG |
| | ACACCGACCACGGGCCGCAGAATCACCATGCCCACCTTGTGAAGC |
| | TGATGGAGTACTGCCAGCATGGCGTGAAGGAAGGGGCCACACTGG |
| | TCTGCGGCGGGAATCAGGTCCCTCGGCCAGGGTTCTTCTTTGAGC |
| | CAACTGTTTTCACAGACGTGGAAGACCACATGTTCATAGCCAAGG |
| | AGGAGTCCTTCGGGCCTGTCATGATCATCTCTCGGTTTGCTGATG |
| | GGGACTTGGATGCCGTGCTGTCTCGGGCCAATGCCACGGAATTTG |
| | GCCTGGCTTCTGGTGTCTTCACCAGGGACATCAACAAGGCCCTGT |
| | ATGTCAGTGACAAGCTCCAGGCAGGCACTGTGTTTGTCAACACGT |
| | ACAACAAGACCGACGTGGCCGCTCCCTTCGGAGGATTCAAACAGT |
| | CTGGATTTGGCAAAGATCTAGGAGAGGCGGCTCTGAACGAGTACC |
| | TGCGGGTCAAGACAGTGACCTTCGAATACTGAAGAAAGGTCTTTG |
| | TGAGAAGAAAGTCCCTGCCCCTCCCTCGTGGCTGGGGCCCCCTCC |
| | CTCTTGAGCCTGGGTGCACAGCACCTCCCACCTGGGGGGCTAGTG |
| | GAAGCCCTCCTGCCTGCACACCATGTCTGCATCTTGGACGCCCTC |
| | TGTCCAGTCAGAAGCAGCCCTTGGCTGGGTGAGGTGTGCCCCTCC |
| | CAGGGAGAATAAAGCTTCTGAAGAGAGACCGTCCACAAAAAAAAA |
| | AAAAAAAAA (SEQ ID NO: 2) |
| Human ALDH1L2 | MLRRGSQALRRFSTGRVYFKNKLKLALIGQSLFGQEVYSHLRKEG |
| Protein Sequence | HRVVGVFTVPDKDGKADPLALAAEKDGTPVFKLPKWRVKGKTIKE |
| mitochondrial 10- | VAEAYRSVGAELNVLPFCTQFIPMDIIDSPKHGSIIYHPSILPRH |
| formyltetrahydro- | RGASAINWTLIMGDKKAGFSVFWADDGLDTGPILLQRSCDVEPND |
| folate dehydrogenase | TVDALYNRFLFPEGIKAMVEAVQLIADGKAPRIPQPEEGATYEGI |
| precursor | QKKENAEISWDQSAEVLHNWIRGHDKVPGAWTEINGQMVTFYGST |
| (NCBI Reference | LLNSSVPPGEPLEIKGAKKPGLVTKNGLVLFGNDGKALTVRNLQF |
| Sequence: | EDGKMIPASQYFSTGETSVVELTAEEVKVAETIKVIWAGILSNVP |
| NP_001029345.2) | IIEDSTDFFKSGASSMDVARLVEEIRQKCGGLQLQNEDVYMATKF |
| | EGFIQKVVRKLRGEDQEVELVVDYISKEVNEIMVKMPYQCFINGQ |
| | FTDADDGKTYDTINPTDGSTICKVSYASLADVDKAVAAAKDAFEN |
| | GEWGRMNARERGRLMYRLADLLEENQEELATIEALDSGAVYTLAL |
| | KTHIGMSVQTFRYFAGWCDKIQGSTIPINQARPNRNLTFTKKEPL |
| | GVCAIIIPWNYPLMMLAWKSAACLAAGNTLVLKPAQVTPLTALKF |
| | AELSVKAGFPKGVINIIPGSGGIAGQRLSEHPDIRKLGFTGSTPI |
| | GKQIMKSCAVSNLKKVSLELGGKSPLIIFNDCELDKAVRMGMGAV |
| | FFNKGENCIAAGRLFVEESIHDEFVTRVVEEIKKMKIGDPLDRST |
| | DHGPQNHKAHLEKLLQYCETGVKEGATLVYGGRQVQRPGFFMEPT |
| | VFTDVEDYMYLAKEESFGPIMVISKFQNGDIDGVLQRANSTEYGL |
| | ASGVFTRDINKAMYVSEKLEAGTVFINTYNKTDVAAPFGGVKQSG |
| | FGKDLGEEALNEYLKTKTVTLEY (SEQ ID NO: 3) |
| Human ALDH1L2 | GCGGCGAGCCGCGAGCCAGGCAGTCCGGGGCATCCAGACTGCAGG |
| mRNA Sequence | CCGCGCCCAGGCCGCGCCCAGGCTGCGCCGCCCGCCTGCCTCCCG |
| mitochondrial 10- | CGCTGCCGCGTCGCCAGTGCTAGCGCTCCTCTCCAGCATGCTGCG |
| formyltetrahydro- | GCGGGGCAGCCAGGCGCTCCGGCGCTTCTCCACTGGCCGGGTTTA |
| folate dehydrogenase | TTTCAAAAACAAGCTGAAGTTGGCACTAATTGGCCAGAGCCTCTT |
| precursor | TGGACAAGAAGTCTATAGCCACCTCCGCAAAGAGGGCCACCGAGT |
| (NCBI Reference | AGTAGGGGTGTTCACAGTTCCAGACAAGGATGGAAAAGCTGACCC |
| Sequence: | TCTGGCTTTGGCTGCAGAGAAAGATGGGACCCCTGTGTTCAAGCT |
| NM_001034173.3) | TCCTAAATGGAGGGTCAAGGGCAAGACCATCAAAGAAGTGGCAGA |
| | AGCCTACAGATCCGTGGGTGCAGAGCTAAATGTGCTCCCTTTCTG |
| | CACTCAGTTCATTCCCATGGATATAATTGATAGTCCAAAGCACGG |
| | CTCTATCATTTATCACCCATCCATCCTGCCCAGGCACAGAGGAGC |
| | CTCTGCTATCAATTGGACTCTAATTATGGGAGATAAGAAAGCTGG |
| | GTTTTCTGTTTTCTGGGCTGATGATGGCTTGGATACAGGACCCAT |
| | CCTTCTTCAGAGATCATGTGATGTTGAACCCAATGATACAGTGGA |
| | TGCACTTTATAATCGGTTTCTTTTTCCTGAAGGAATCAAGGCCAT |
| | GGTAGAAGCTGTCCAACTCATAGCTGATGGAAAAGCTCCTCGTAT |
| | ACCCCAGCCAGAAGAAGGGGCAACATATGAAGGTATCCAGAAAAA |
| | GGAAAATGCTGAGATTTCTTGGGACCAGTCTGCCGAAGTTTTACA |
| | TAACTGGATTCGAGGTCATGATAAAGTCCCTGGAGCTTGGACAGA |
| | GATAAATGGACAGATGGTCACTTTCTATGGCTCGACATTACTGAA |
| | TAGCTCTGTGCCTCCTGGAGAACCACTGGAAATTAAAGGTGCCAA |
| | GAAGCCTGGTCTCGTTACCAAAAATGGACTTGTTCTTTTTGGTAA |
| | CGATGGAAAAGCACTGACGGTGAGAAATCTGCAGTTTGAAGATGG |
| | AAAAATGATCCCTGCCTCTCAGTACTTTTCAACGGGTGAGACGTC |
| | AGTGGTAGAACTGACAGCTGAAGAGGTGAAAGTGGCAGAGACCAT |
| | CAAGGTCATCTGGGCTGGAATTTTAAGCAATGTCCCCATTATTGA |
| | AGACTCAACAGACTTCTTTAAATCTGGAGCAAGCTCAATGGATGT |
| | TGCCAGGCTGGTTGAAGAGATCAGACAGAAATGTGGTGGGCTTCA |
| | GTTGCAGAATGAAGATGTCTATATGGCCACCAAGTTTGAAGGCTT |
| | TATCCAAAAGGTCGTGAGGAAACTGAGAGGAGAAGATCAAGAGGT |
| | GGAGCTGGTTGTAGATTATATTTCAAAGGAGGTCAATGAAATCAT |
| | GGTAAAAATGCCATACCAGTGTTTCATAAATGGACAGTTCACAGA |
| | TGCAGACGATGGAAAGACTTACGACACTATCAACCCAACAGATGG |
| | ATCTACAATATGCAAAGTATCCTACGCTTCTTTGGCGGATGTTGA |
| | TAAAGCAGTAGCAGCAGCAAAAGATGCTTTTGAAAACGGTGAATG |
| | GGGAAGAATGAATGCAAGAGAAAGAGGAAGATTGATGTATAGACT |
| | TGCAGACCTACTGGAAGAGAACCAAGAAGAGCTGGCAACTATTGA |
| | AGCCCTTGATTCAGGGGCTGTCTATACCTTGGCCCTGAAGACACA |
| | CATTGGAATGTCTGTGCAAACATTCAGATATTTTGCTGGCTGGTG |
| | CGACAAAATTCAGGGTTCTACTATTCCAATCAACCAGGCCCGTCC |
| | AAATCGCAATCTGACCTTCACCAAGAAAGAGCCACTCGGTGTCTG |
| | TGCCATTATTATTCCCTGGAACTACCCGCTGATGATGCTGGCATG |
| | GAAGAGTGCTGCGTGTTTGGCAGCAGGCAATACCTTAGTGCTCAA |
| | GCCAGCACAGGTCACGCCCTTGACTGCTTTGAAGTTTGCAGAACT |
| | GTCTGTGAAAGCAGGCTTTCCAAAGGGGGTCATCAACATCATTCC |
| | AGGCTCAGGTGGCATAGCAGGACAACGTCTGTCTGAACATCCTGA |
| | CATCCGCAAACTTGGTTTCACTGGATCCACTCCTATTGGCAAACA |
| | GATCATGAAGAGCTGTGCTGTTAGCAACTTGAAGAAAGTTTCCCT |
| | TGAGCTTGGTGGCAAGTCTCCACTTATAATATTTAATGACTGTGA |
| | ACTTGACAAGGCTGTGCGAATGGGCATGGGAGCAGTATTTTTCAA |
| | CAAAGGAGAGAACTGTATTGCTGCTGGGCGGTTGTTCGTGGAAGA |
| | ATCCATCCACGACGAATTTGTGACAAGAGTGGTAGAAGAAATTAA |
| | AAAGATGAAAATTGGTGATCCACTTGACAGATCCACTGATCATGG |
| | GCCCCAAAATCATAAGGCTCATCTGGAAAAGCTGCTGCAATACTG |
| | TGAAACTGGAGTGAAAGAAGGGGCCACTTTGGTGTACGGGGGAAG |
| | ACAAGTCCAAAGGCCAGGCTTTTTCATGGAGCCGACCGTGTTCAC |
| | AGATGTGGAAGACTACATGTACCTCGCCAAAGAGGAATCCTTTGG |
| | GCCTATTATGGTCATTTCTAAATTCCAAAATGGGGACATCGATGG |
| | AGTGTTGCAGCGAGCAAATAGTACAGAGTATGGTTTGGCCTCAGG |
| | GGTTTTTACAAGAGACATAAACAAAGCTATGTATGTGAGTGAAAA |
| | ACTGGAAGCAGGAACTGTTTTTATTAACACATACAACAAGACAGA |
| | TGTGGCGGCCCCATTTGGCGGAGTTAAACAATCTGGCTTTGGAAA |
| | AGACTTAGGTGAGGAAGCTCTAAATGAATATCTCAAAACCAAGAC |
| | GGTGACACTGGAATATTAGAGCAACACCATCATCAGGAAAGCCTT |
| | GACAGACAGCCCTTTACAACTCTGGACACACTTAAGAAGATTGGG |
| | TGTGTTGAGGCAGGAGGTGTCAGCCACAAACCAAAAAATACACAG |
| | ATGGACCATGAAGAGGGCCAGGCCATGTTAAAGCATTTACACATG |
| | TGCCTGAGTATTTTCTAATACACCTTCCAGTGATTTGGAGTTGTT |
| | GCATTTTGACTATGTTGTATATCATACGTATTTCTAAAATACCAA |
| | GCTGTTTCTCCCCTACCTAGACAAATCTATTCATGGTTCCCATCT |
| | TGAAGATGTCAGTACCATGCAGTTATAATACACAAGGTGCATTTA |
| | TTGGAAACTTTGTATAATATGTACAGGTTTTTAACCTCTGAACTA |
| | TACATAGGGGGTTATTAAAAAGATTTTCTATAAGTCTTCTAAGGA |
| | ACAGTATAACCTGTAAGGAATGTGAAGGTAGTTCTTTTTTAGTAT |
| | TTGGAAATAAGATACATCTTTGTGCCTTTGATATTCCATTTTTTA |
| | ACCCACTGTGATGGGTGATCAACCTAGAAACATTATCTTGAGTAC |
| | CTACTAGGTACCAGGTACTATATTATGTTCTGAGGAGTATAGAGA |
| | ATTTAATGATATGATGGCTGGCCCCCACATAGTTTAAATTTTAGT |
| | AAATAGCTTTTGAAGCAAATTTTACATATGATATAGTAGAAGGCT |
| | GATCCTGGTCGTATCATACCATCTTCCTATCTATGTAACTTTGGG |
| | AAACTCTCGCAACTCCTCTGAGCCTCTGCTTCCCTATGTGTAAAA |
| | CAGGGATAGTAAATGCCTTCCTCAGGACCCTTAATAGGAGAATTC |
| | ATTGCAGTAATGTAAGTAAAGCACCTCACATTAATGCTTTGCTCA |
| | TGGTAAGTACTCAAATTTAACTCTGATTTCCTCCGTCACCATTCT |
| | TAAAAGATATTGAGATAGTTTAATTAACTAGATGAATTCATTTCC |
| | CACAACCCTTTTCAATCATCAATTCCTAGATATTTTTCTCATCCA |
| | TTGTTCTGACACAATGCCTGATACAGCAGCACTGAAAAATGCCAC |
| | ACAATGAAAAATGGCAATAGTACAAGGAAAAGGGGTGCTTTTCTT |
| | TGGGCAGCTCGCTCGTCCTTCATGGGACATCTTACTTTCCATTTT |
| | TCTACCTATTGGTTCTGCTGTTCACTGGCTGTGTGATCTTGGGCA |
| | AGATAGTAATCTAATATCTCAGAGCCTAGGTTGAGTATCTATAAA |
| | ATGAAAATCAAATCTCTATCTCAGTAGGTGTTGCAAGGATTCAGT |
| | GAGATAATATACATAATGCACTTAACAAGGCGTTTGGACCATAGC |
| | ATTGAAGAAATGGAAACTATTAACAGCCCATTTCCCATTGGCAGA |
| | CAGAAGTAGTCAGGTGAGTAAATTTTCACCATCTATGTGTGACTA |
| | GAAGGCGGCAAATTTCTGAATCACATGAGTCTCCAAAAGATAGCC |
| | AGAAAGTTAAATTCTATTAATCCTCCTTTAAAAATAAAATTTCAG |
| | TAAACATTCCTTTTTCTTTGGCTTTGAAGAAGCCTTAGGGAATAT |
| | TTGTCATTTTGGAGACTTGGCAGAATAACATGAGGGGATTGTAGG |
| | GAATCAATAAAAACTAAACAACAAAATCAGAGTCAGAGAACATTT |
| | TCAAAAGGAAGAATAGGAGGTTTGATCCCAGCATGATAAACAGAG |
| | CGAATTTGGCCTGGAAGCACTTTTGATTATACTATAGCTCATTTA |
| | CCATCCCAGAGTTTGGCACAGCTGAAATTTTAAGTTGGAATGAAT |
| | ATTCACTGGGCCCAAAATGACAGTTCATATTTGAATAAAAGTGAC |
| | AAAAGCCTTTTTATAAGTAATCACTTTTAAGTGAAATGTTTTAAC |
| | TGATTTCATGTGATTTAGAATATGATTTAATCAAATTATTTTAAT |
| | GATAGATGGAATGGCAGACAAAAACATGCCTGTCCTTCTAGACTG |
| | ATTTTACTTTACCCTCTAATATTCATCTCAGTAGCAGTGTTTTAA |
| | ATATTCTCTGGGCTGCAAAACTCTTTGGGAATCTGATAAAAGCTA |
| | TGAACACTCCCTGTGTCCCGCTTCTACCCCCAAAATTCATGTGCA |
| | CACACACAATTCTGCAAGTATCTTCAAAGGGTTCACAGACCTCCC |
| | AAAGGCCATGCTTGGGCCCCAGATTAAGAACTCCTTTCTCCATAG |
| | CAAGTTTTAAACATTTCTTACCAGCTTACATTTTTAGATCTGGCT |
| | GATCAGAATCAAAGGCTCTGTGTAATACATAAAGTTACCAAGTGA |
| | ACTGGAATTGGAACATCACCCTCCCCAGCCTGCTAGGTGATTTAC |
| | TTAACACATAGAGTAATAAAATCATCGCTGTTGCTTTAGATCACG |
| | GATTATTTTGCTAATAATGCTAAGGATGAAGCTGTGATCTTATTA |
| | TCACCTGAATCGGGAGGTGTGGACACTTTAAGCAGTTCCACTTTC |
| | CTTCTAATTCCCCATCCCCATGCCTTTGCTAAAGCTGTCCCTTTT |
| | GCTCTAACACCCTTCCTGGACCTTCCTACCCTAGCTGGGCTAAGT |
| | GTTTCTCCTCAGCGTTCCCACTTGTTTCAAACATAGCACTTACCA |
| | CTTGTACTAAAATTACTTGCCTTCTTAATTAGATATGAACAACCC |
| | TCCCCAACTCCAGTATGGGCCTTCTGTCAATAATAATACGATATG |
| | ACAGCTACCATTTATTAAGGGCCTCCTGTATGAAAGACCTTAGGC |
| | TAAGCATGTTTTAAATGTTATTTAATCTTCACAATCTCTGAAAAA |
| | AATGAAGAAATCAACGTGCTTTTCTTACTACCTCTACCCCTAAGC |
| | CATTATTACTTTTTTTTTTTTTTGAGACAGAGTTTTGCTCTTGTT |
| | GCCCAGGCTGCAGTGCAGTGGTGCAATCTTGGCTCACTGCAACCT |
| | CTGCCTCTTGGGTTCAAGCGATTGTCATGCCTTAGCCTTCCAAGT |
| | AGCTGGGATTACAGGTGTGTGCCACTACACCTGGCTAAGTAGAGA |
| | TGGGGTTTCGCCATGTTGGCCAGGCTGGTCTTGAACTCCTGACCT |
| | CAAGTGATCCACCTGCCTCCGCCTCCCAAAGTGCTGGGATTACAG |
| | GCATGAACCACTGCACCTGGCCTGTTACCTCTTTCCTACAATTTT |
| | GCTCAAGTCTCCCAACTGGTCTTCTGGATTCCTCTCTTCTGCGGT |
| | CCTGTTCAAAGCTTAAGTCAGACAGTGTCACTTCACTCGTCTGTT |
| | TAAAACCTTTCAATGGCCCCCATTTCACGTAGACCAAAGTCCAAC |
| | GTATTTACCTGGCCTACTGATCTTGCTCCTAGCTACCTCTGACCT |
| | CATCTCCTGTCAATTTCCCTCTCATTCTGTTCCACCATCCTGACT |
| | GCCTTGACTTCCTCAACAGAACAAGCCTGCTCCTGCCTCAGGGCC |
| | TCTGTCCTTATTCTTCCTCTTCCCAGGGGTGTGCTGGTAAAATAT |
| | TTAACAAATAGTTCTCCGGGACGGGGGAGAAAACCCTCATTTGTA |
| | GCATTTGCAGGTATCTATGTGTAAATACTCTCATCAAGGCTATTT |
| | TTGAGCCACTAATTTGCCTTCACTGAATACAGAGTTTGGGAAGAG |
| | ATGCATGCCATCAGAACAAATGCAAGCCAGCACCAGCACACCACT |
| | GCCTCTTCCTGCAACTCTTGTCCATACACAACCTCATGGCTGGCT |
| | GGCTCACTTCCTGCAGGTCTCTCCTCAAATATCATCTGATGAGAG |
| | ACACATTCCCTGACTATGCTTTCTAAAATAGGCCATATGCCCCCA |
| | CATTCATACCCCATCTGCTGTCATTCTTTATTCTTTTTATAAGTG |
| | CATTATTTTCATAGCACTTATCACTACCTGTTGTATATTAATCAA |
| | TGATCTTTTCCCATTAGAATGTAAGTTTCATGAACAGGTACTTGT |
| | TTTAATACTGTATCTCCAGTCCTAATGTGTAACAGGAGCCCAATA |
| | AATGTTTGCTTTCAAATGGAGAGGTTAAGTAACCTGCTCAAATCA |
| | CACAGCTATTAAGTGGCAGAACAGGTTTTCAAGCAATGCATCTGG |
| | TGGTTTTAACTAAGTCGAGATAGTTTTTATTCCTAATGCCTAAAT |
| | CAGGGCCTAGGTAGTGAGCTGTGGGCACATATTAAGTATTGGTTA |
| | AACTAAAAATAATAAGCAAAATGGACATTATCTATAAAAGCTTTT |
| | GTGGAAATGGCTAGAGCTAGGGTAAGGAAACAAATTTGGTTCCCC |
| | ATACCTGCCCTCCAAGAAAATAAAGCTGTCAAGGAAAATCTGGGC |
| | TAAGAGTAGGATATGAGGGATGATGGATAAGGCATGAGACATGAG |
| | AAATAAGGGGGATTAAATTATTATTACTATTATACAAATGATGCC |
| | TGAGTAGATTTTTAAAATGATTAAATACCCAATGATGTAAAAAAC |
| | ATTTATAAAATAGGAAAGTAAGACTGACTCAACCATAATTTGTTG |
| | AGTCAACCCAAAAATCTATTTGGTTATTTTCAAACAGAAATAGCC |
| | TACAGATGATATCTGAGATTGTTCCAAACTTTTTCTATGAATATG |
| | TATACTTTTTTTACATAATTAACATAATACTGTATATTAATTTGT |
| | TACCTGCTTTTTCAATTAACAATATATCATAAGCATCTATGCCAA |
| | TAAACACAATTCTGCATATTTCAAAAAAAAAAAAAAAAAA |
| | (SEQ ID NO: 4) |
| Human FOLR1 | MAQRMTTQLLLLLVWVAVVGEAQTRIAWARTELLNVCMNAKHHKE |
| Protein Sequence | KPGPEDKLHEQCRPWRKNACCSTNTSQEAHKDVSYLYRFNWNHCG |
| folate receptor | EMAPACKRHFIQDTCLYECSPNLGPWIQQVDQSWRKERVLNVPLC |
| alpha precursor | KEDCEQWWEDCRTSYTCKSNWHKGWNWTSGFNKCAVGAACQPFHF |
| (NCBI Reference | YFPTPTVLCNEIWTHSYKVSNYSRGSGRCIQMWFDPAQGNPNEEV |
| Sequence: | ARFYAAAMSGAGPWAAWPFLLSLALMLLWLLS (SEQ ID |
| NP_057936.1) | NO: 5) |
| Human FOLR1 | TGGAGGCCTGGCTGGTGCTCACATACAATAATTAACTGCTGAGTG |
| mRNA Sequence | GCCTTCGCCCAATCCCAGGCTCCACTCCTGGGCTCCATTCCCACT |
| Homo sapiens folate | CCCTGCCTGTCTCCTAGGCCACTAAACCACAGCTGTCCCCTGGAA |
| receptor 1 (adult) | TAAGGCAAGGGGGAGTGTAGAGCAGAGCAGAAGCCTGAGCCAGAC |
| (FOLR1), transcript | GGAGAGCCACCTCCTCTCCCAGGAACTGAACCCAAAGGATCACCT |
| variant 7, mRNA | GGTATTCCCTGAGAGTACAGATTTCTCCGGCGTGGCCCTCAAGGG |
| (NCBI Reference | ACAGACATGGCTCAGCGGATGACAACACAGCTGCTGCTCCTTCTA |
| Sequence: | GTGTGGGTGGCTGTAGTAGGGGAGGCTCAGACAAGGATTGCATGG |
| NM_016724.2) | GCCAGGACTGAGCTTCTCAATGTCTGCATGAACGCCAAGCACCAC |
| | AAGGAAAAGCCAGGCCCCGAGGACAAGTTGCATGAGCAGTGTCGA |
| | CCCTGGAGGAAGAATGCCTGCTGTTCTACCAACACCAGCCAGGAA |
| | GCCCATAAGGATGTTTCCTACCTATATAGATTCAACTGGAACCAC |
| | TGTGGAGAGATGGCACCTGCCTGCAAACGGCATTTCATCCAGGAC |
| | ACCTGCCTCTACGAGTGCTCCCCCAACTTGGGGCCCTGGATCCAG |
| | CAGGTGGATCAGAGCTGGCGCAAAGAGCGGGTACTGAACGTGCCC |
| | CTGTGCAAAGAGGACTGTGAGCAATGGTGGGAAGATTGTCGCACC |
| | TCCTACACCTGCAAGAGCAACTGGCACAAGGGCTGGAACTGGACT |
| | TCAGGGTTTAACAAGTGCGCAGTGGGAGCTGCCTGCCAACCTTTC |
| | CATTTCTACTTCCCCACACCCACTGTTCTGTGCAATGAAATCTGG |
| | ACTCACTCCTACAAGGTCAGCAACTACAGCCGAGGGAGTGGCCGC |
| | TGCATCCAGATGTGGTTCGACCCAGCCCAGGGCAACCCCAATGAG |
| | GAGGTGGCGAGGTTCTATGCTGCAGCCATGAGTGGGGCTGGGCCC |
| | TGGGCAGCCTGGCCTTTCCTGCTTAGCCTGGCCCTAATGCTGCTG |
| | TGGCTGCTCAGCTGACCTCCTTTTACCTTCTGATACCTGGAAATC |
| | CCTGCCCTGTTCAGCCCCACAGCTCCCAACTATTTGGTTCCTGCT |
| | CCATGGTCGGGCCTCTGACAGCCACTTTGAATAAACCAGACACCG |
| | CACATGTGTCTTGAGAATTATTTGGAAAAAAAAAAAAAAAAAA |
| | (SEQ ID NO: 6) |
| Human FPGS | MSRARSHLRAALFLAAASARGITTQVAARRGLSAWPVPQEPSMEY |
| Protein Sequence | QDAVRMLNTLQTNAGYLEQVKRQRGDPQTQLEAMELYLARSGLQV |
| folylpolyglutamate | EDLDRLNIIHVTGTKGKGSTCAFTECILRSYGLKTGFFSSPHLVQ |
| synthase, | VRERIRINGQPISPELFTKYFWRLYHRLEETKDGSCVSMPPYFRF |
| mitochondrial | LTLMAFHVFLQEKVDLAVVEVGIGGAYDCTNIIRKPVVCGVSSLG |
| isoform a precursor | IDHTSLLGDTVEKIAWQKGGIFKQGVPAFTVLQPEGPLAVLRDRA |
| (NCBI Reference | QQISCPLYLCPMLEALEEGGPPLTLGLEGEHQRSNAALALQLAHC |
| Sequence: | WLQRQDRHGAGEPKASRPGLLWQLPLAPVFQPTSHMRLGLRNTEW |
| NP_004948.4) | PGRTQVLRRGPLTWYLDGAHTASSAQACVRWFRQALQGRERPSGG |
| | PEVRVLLFNATGDRDPAALLKLLQPCQFDYAVFCPNLTEVSSTGN |
| | ADQQNFTVTLDQVLLRCLEHQQHWNHLDEEQASPDLWSAPSPEPG |
| | GSASLLLAPHPPHTCSASSLVFSCISHALQWISQGRDPIFQPPSP |
| | PKGLLTHPVAHSGASILREAAAIHVLVTGSLHLVGGVLKLLEPAL |
| | SQ (SEQ ID NO: 7) |
| Human FPGS | GCGGGGCGTCTCCCGCCCGGGCCTAGAGCGCTGCCGGGGGCGCCG |
| mRNA Sequence | GGACTATGTCGCGGGCGCGGAGCCACCTGCGCGCCGCTCTATTCC |
| folylpolyglutamate | TGGCAGCGGCGTCTGCGCGCGGCATAACGACCCAGGTCGCGGCGC |
| synthase (FPGS), | GGCGGGGCTTGAGCGCGTGGCCGGTGCCGCAGGAGCCGAGCATGG |
| nuclear gene | AGTACCAGGATGCCGTGCGCATGCTCAATACCCTGCAGACCAATG |
| encoding | CCGGCTACCTGGAGCAGGTGAAGCGCCAGCGGGGTGACCCTCAGA |
| mitochondrial | CACAGTTGGAAGCCATGGAACTGTACCTGGCACGGAGTGGGCTGC |
| protein, transcript | AGGTGGAGGACTTGGACCGGCTGAACATCATCCACGTCACTGGGA |
| variant 1 | CGAAGGGGAAGGGCTCCACCTGTGCCTTCACGGAATGTATCCTCC |
| (NCBI Reference | GAAGCTATGGCCTGAAGACGGGATTCTTTAGCTCTCCCCACCTGG |
| Sequence: | TGCAGGTTCGGGAGCGGATCCGCATCAATGGGCAGCCCATCAGTC |
| NM_004957.4) | CTGAGCTCTTCACCAAGTACTTCTGGCGCCTCTACCACCGGCTGG |
| | AGGAGACCAAGGATGGCAGCTGTGTCTCCATGCCCCCCTACTTCC |
| | GCTTCCTGACACTCATGGCCTTCCACGTCTTCCTCCAAGAGAAGG |
| | TGGACCTGGCAGTGGTGGAGGTGGGCATTGGCGGGGCTTATGACT |
| | GCACCAACATCATCAGGAAGCCTGTGGTGTGCGGAGTCTCCTCTC |
| | TTGGCATCGACCACACCAGCCTCCTGGGGGATACGGTGGAGAAGA |
| | TCGCATGGCAGAAAGGGGGCATCTTTAAGCAAGGTGTCCCTGCCT |
| | TCACTGTGCTCCAACCTGAAGGTCCCCTGGCAGTGCTGAGGGACC |
| | GAGCCCAGCAGATCTCATGTCCTCTATACCTGTGTCCGATGCTGG |
| | AGGCCCTCGAGGAAGGGGGGCCGCCGCTGACCCTGGGCCTGGAGG |
| | GGGAGCACCAGCGGTCCAACGCCGCCTTGGCCTTGCAGCTGGCCC |
| | ACTGCTGGCTGCAGCGGCAGGACCGCCATGGTGCTGGGGAGCCAA |
| | AGGCATCCAGGCCAGGGCTCCTGTGGCAGCTGCCCCTGGCACCTG |
| | TGTTCCAGCCCACATCCCACATGCGGCTCGGGCTTCGGAACACGG |
| | AGTGGCCGGGCCGGACGCAGGTGCTGCGGCGCGGGCCCCTCACCT |
| | GGTACCTGGACGGTGCGCACACCGCCAGCAGCGCGCAGGCCTGCG |
| | TGCGCTGGTTCCGCCAGGCGCTGCAGGGCCGCGAGAGGCCGAGCG |
| | GTGGCCCCGAGGTTCGAGTCTTGCTCTTCAATGCTACCGGGGACC |
| | GGGACCCGGCGGCCCTGCTGAAGCTGCTGCAGCCCTGCCAGTTTG |
| | ACTATGCCGTCTTCTGCCCTAACCTGACAGAGGTGTCATCCACAG |
| | GCAACGCAGACCAACAGAACTTCACAGTGACACTGGACCAGGTCC |
| | TGCTCCGCTGCCTGGAACACCAGCAGCACTGGAACCACCTGGACG |
| | AAGAGCAGGCCAGCCCGGACCTCTGGAGTGCCCCCAGCCCAGAGC |
| | CCGGTGGGTCCGCATCCCTGCTTCTGGCGCCCCACCCACCCCACA |
| | CCTGCAGTGCCAGCTCCCTCGTCTTCAGCTGCATTTCACATGCCT |
| | TGCAATGGATCAGCCAAGGCCGAGACCCCATCTTCCAGCCACCTA |
| | GTCCCCCAAAGGGCCTCCTCACCCACCCTGTGGCTCACAGTGGGG |
| | CCAGCATACTCCGTGAGGCTGCTGCCATCCATGTGCTAGTCACTG |
| | GCAGCCTGCACCTGGTGGGTGGTGTCCTGAAGCTGCTGGAGCCCG |
| | CACTGTCCCAGTAGCCAAGGCCCGGGGTTGGAGGTGGGAGCTTCC |
| | CACACCTGCCTGCGTTCTCCCCATGAACTTACATACTAGGTGCCT |
| | TTTGTTTTTGGCTTTCCTGGTTCTGTCTAGACTGGCCTAGGGGCC |
| | AGGGCTTTGGGATGGGAGGCCGGGAGAGGATGTCTTTTTTAAGGC |
| | TCTGTGCCTTGGTCTCTCCTTCCTCTTGGCTGAGATAGCAGAGGG |
| | GCTCCCCGGGTCTCTCACTGTTGCAGTGGCCTGGCCGTTCAGCCT |
| | GTCTCCCCCAACACCCCGCCTGCCTCCTGGCTCAGGCCCAGCTTA |
| | TTGTGTGCGCTGCCTGGCCAGGCCCTGGGTCTTGCCATGTGCTGG |
| | GTGGTAGATTTCCTCCTCCCAGTGCCTTCTGGGAAGGGAGAGGGC |
| | CTCTGCCTGGGACACTGCGGGACAGAGGGTGGCTGGAGTGAATTA |
| | AAGCCTTTGTTTTTTAAAGAAATGGCAAAGCCTTCGACTGACCCT |
| | TGACCCCCTGCTCCCTCAGCAGAGACGGAGGGAGGGGCTGCTGGT |
| | GGTCAGGGACCTGCACTGTGTAGAGGGAGCCTGGCTGTGTGGCCT |
| | GGAACAAGTCCCTCCCTCCCTGTGCGCCTCAGGTGGCCTGTCTGT |
| | GAGATGAGAAGAAGACCAGACTGAAGCCTGTTCACCATATGCCAG |
| | GCAGTGCTTTCT (SEQ ID NO: 8) |
| Human GCSH | MALRVVRSVRALLCTLRAVPSPAAPCPPRPWQLGVGAVRTLRTGP |
| Protein Sequence | ALLSVRKFTEKHEWVTTENGIGTVGISNFAQEALGDVVYCSLPEV |
| glycine cleavage | GTKLNKQDEFGALESVKAASELYSPLSGEVTEINEALAENPGLVN |
| system H protein, | KSCYEDGWLIKMTLSNPSELDELMSEEAYEKYIKSIEE (SEQ |
| mitochondrial | ID NO: 9) |
| precursor | |
| (NCBI Reference | |
| Sequence: NP_ | |
| NP_004474.2) | |
| Human GCSH | CAGCCGGCTCCCTCCGGCCGCGAACTGCCCCTCCCCGCCCCGCCT |
| mRNA Sequence | CCCGGCGCGGGTGGCCGAGGCGTAGCGCTGCGACCCCCGCACCCC |
| glycine cleavage | TGCGAACATGGCGCTGCGAGTGGTGCGGAGCGTGCGGGCCCTGCT |
| system protein H | CTGCACCCTGCGCGCGGTCCCGTCACCCGCCGCGCCCTGCCCGCC |
| (aminomethyl | GAGGCCCTGGCAGCTGGGGGTGGGCGCCGTCCGTACGCTGCGCAC |
| carrier) | TGGACCCGCTCTGCTCTCGGTGCGTAAATTCACAGAGAAACACGA |
| (NCBI Reference | ATGGGTAACAACAGAAAATGGCATTGGAACAGTGGGAATCAGCAA |
| Sequence: | TTTTGCACAGGAAGCGTTGGGAGATGTTGTTTATTGTAGTCTCCC |
| NM_004483.4) | TGAAGTTGGGACAAAATTGAACAAACAAGATGAGTTTGGTGCTTT |
| | GGAAAGTGTGAAAGCTGCTAGTGAACTCTATTCTCCTTTATCAGG |
| | AGAAGTAACTGAAATTAATGAAGCTCTTGCAGAAAATCCAGGACT |
| | TGTAAACAAATCTTGTTATGAAGATGGTTGGCTGATCAAGATGAC |
| | ACTGAGTAACCCTTCAGAACTAGATGAACTTATGAGTGAAGAAGC |
| | ATATGAGAAATACATAAAATCTATTGAGGAGTGAAAATGGAACTC |
| | CTAAATAAACTAGTATGAAATAACGCAAGCCAGCAGAGTTGTCTT |
| | AAATTAGTGGTGGATAGAAGACTTAGAATAGAAACTTTTAGTATT |
| | ACCGATGGGGAAAAAAAAACTACTGTTAACACTGCTAATGAAAGA |
| | AAATGCCCTTTAACTTTCTAATGATTATAGATAAATATAATATGC |
| | GTCTTTTTCACAATATCCTATGATTTTTAGACTAGGCTCTAGTGT |
| | TCAGAATTCATGAAATTATCCATGGTAAAAACTAGTTATAAAAAT |
| | TACATAATTCAAAGATAACATTGTTATTCTTAAGCCTTATATAAT |
| | ATTGTAACTTGCATGTATCCATACCTGGATTTGGGATGAAATACT |
| | TAATGATCTTTCCATTGGAAATAACTGGAAGTGAAGAGGTTTTGT |
| | TGCTTGTACAGTGTCAGATGAGGAACACCACTATCTTAATTTTGC |
| | GATACACTGCATTTGCTGGTGCTATTTTTATACAGTGAAGCAACA |
| | GCTTTGCAGCAAAATAATAAAATACTTCTTCGTTAATCATGTTTG |
| | TTTTGATGTTAATATTTCATTTAGTAACTCTGCTAGTATTTGTGA |
| | AAGTGCTAACTTTAACTTACGGAAAGTTACTTTTTAAAAGGAAAT |
| | TTAAGCCAGAACAATGCAAAGCTCCAAGAAAATGTTTTCTTTAGT |
| | CACAAATCTGGTTTTTCTTAAGCCAAGATCTGTCACCTTTAACAT |
| | AATAAAAAATAAATCACCAACTTTGATTTTCTATCATGCGAGGTC |
| | TGAAGAAAGAAGAGGAAAGACAGAGGAAGGTGGAAGTTTTGATCA |
| | GTATAGCACATGGTGTTTTTAAGTTGTTAAACCACGTTCAGGTTT |
| | CCACTTAAGTCATGGGAATAAAAGTGGACAAGGACTGAAGCTTTA |
| | TGAGCTCA (SEQ ID NO: 10) |
| Human GLDC | MQSCARAWGLRLGRGVGGGRRLAGGSGPCWAPRSRDSSSGGGDSA |
| Protein Sequence | AAGASRLLERLLPRHDDFARRHIGPGDKDQREMLQTLGLASIDEL |
| glycine | IEKTVPANIRLKRPLKMEDPVCENEILATLHAISSKNQIWRSYIG |
| dehydrogenase | MGYYNCSVPQTILRNLLENSGWITQYTPYQPEVSQGRLESLLNYQ |
| [decarboxylating], | TMVCDITGLDMANASLLDEGTAAAEALQLCYRHNKRRKFLVDPRC |
| mitochondrial | HPQTIAVVQTRAKYTGVLTELKLPCEMDFSGKDVSGVLFQYPDTE |
| precursor | GKVEDFTELVERAHQSGSLACCATDLLALCILRPPGEFGVDIALG |
| (NCBI Reference | SSQRFGVPLGYGGPHAAFFAVRESLVRMMPGRMVGVTRDATGKEV |
| Sequence: | YRLALQTREQHIRRDKATSNICTAQALLANMAAMFAIYHGSHGLE |
| NP_000161.2) | HIARRVHNATLILSEGLKRAGHQLQHDLFFDTLKIQCGCSVKEVL |
| | GRAAQRQINFRLFEDGTLGISLDETVNEKDLDDLLWIFGCESSAE |
| | LVAESMGEECRGIPGSVFKRTSPFLTHQVFNSYHSETNIVRYMKK |
| | LENKDISLVHSMIPLGSCTMKLNSSSELAPITWKEFANIHPFVPL |
| | DQAQGYQQLFRELEKDLCELTGYDQVCFQPNSGAQGEYAGLATIR |
| | AYLNQKGEGHRTVCLIPKSAHGTNPASAHMAGMKIQPVEVDKYGN |
| | IDAVHLKAMVDKHKENLAAIMITYPSTNGVFEENISDVCDLIHQH |
| | GGQVYLDGANMNAQVGICRPGDFGSDVSHLNLHKTFCIPHGGGGP |
| | GMGPIGVKKHLAPFLPNHPVISLKRNEDACPVGTVSAAPWGSSSI |
| | LPISWAYIKMMGGKGLKQATETAILNANYMAKRLETHYRILFRGA |
| | RGYVGHEFILDTRPFKKSANIEAVDVAKRLQDYGFHAPTMSWPVA |
| | GTLMVEPTESEDKAELDRFCDAMISIRQEIADIEEGRIDPRVNPL |
| | KMSPHSLTCVTSSHWDRPYSREVAAFPLPFVKPENKFWPTIARID |
| | DIYGDQHLVCTCPPMEVYESPFSEQKRASS (SEQ ID NO: |
| | 11) |
| Human GLDC | CTTTGCGCGAGTGTCTTGGTTGAGCGCAGCGCCCATTCATTGCCC |
| mRNA Sequence | GCGAGCGTCCATCCATCTGTCCGGCCGACTGTCCAGCGAAAGGGG |
| glycine | CTCCAGGCCGGGCGCAGCCGCCACCCGGGGGACCGAGGCCAGGAG |
| dehydrogenase | AGGGGCCAAGAGCGCGGCTGACCCTTGCGGGCCGGGGCAGGGGAC |
| (decarboxylating) | GGTGGCCGCGGCCATGCAGTCCTGTGCCAGGGCGTGGGGGCTGCG |
| (GLDC), nuclear | CCTGGGCCGCGGGGTCGGGGGCGGCCGCCGCCTGGCTGGGGGATC |
| gene encoding | GGGGCCGTGCTGGGCGCCGCGGAGCCGGGACAGCAGCAGTGGCGG |
| mitochondrial, | CGGGGACAGCGCCGCGGCTGGGGCCTCGCGCCTCCTGGAGCGCCT |
| (NCBI Reference | TCTGCCCAGACACGACGACTTCGCTCGGAGGCACATCGGCCCTGG |
| Sequence: | GGACAAAGACCAGAGAGAGATGCTGCAGACCTTGGGGCTGGCGAG |
| NM_000170.2) | CATTGATGAATTGATCGAGAAGACGGTCCCTGCCAACATCCGTTT |
| | GAAAAGACCCTTGAAAATGGAAGACCCTGTTTGTGAAAATGAAAT |
| | CCTTGCAACTCTGCATGCCATTTCAAGCAAAAACCAGATCTGGAG |
| | ATCGTATATTGGCATGGGCTATTATAACTGCTCAGTGCCACAGAC |
| | GATTTTGCGGAACTTACTGGAGAACTCAGGATGGATCACCCAGTA |
| | TACTCCATACCAGCCTGAGGTGTCTCAGGGGAGGCTGGAGAGTTT |
| | ACTCAACTACCAGACCATGGTGTGTGACATCACAGGCCTGGACAT |
| | GGCCAATGCATCCCTGCTGGATGAGGGGACTGCAGCCGCAGAGGC |
| | ACTGCAGCTGTGCTACAGACACAACAAGAGGAGGAAATTTCTCGT |
| | TGATCCCCGTTGCCACCCACAGACAATAGCTGTTGTCCAGACTCG |
| | AGCCAAATATACTGGAGTCCTCACTGAGCTGAAGTTACCCTGTGA |
| | AATGGACTTCAGTGGAAAAGATGTCAGTGGAGTGTTGTTCCAGTA |
| | CCCAGACACGGAGGGGAAGGTGGAAGACTTTACGGAACTCGTGGA |
| | GAGAGCTCATCAGAGTGGGAGCCTGGCCTGCTGTGCTACTGACCT |
| | TTTAGCTTTGTGCATCTTGAGGCCACCTGGAGAATTTGGGGTAGA |
| | CATCGCCCTGGGCAGCTCCCAGAGATTTGGAGTGCCACTGGGCTA |
| | TGGGGGACCCCATGCAGCATTTTTTGCTGTCCGAGAAAGCTTGGT |
| | GAGAATGATGCCTGGAAGAATGGTGGGGGTAACAAGAGATGCCAC |
| | TGGGAAAGAAGTGTATCGTCTTGCTCTTCAAACCAGGGAGCAACA |
| | CATTCGGAGAGACAAGGCTACCAGCAACATCTGTACAGCTCAGGC |
| | CCTCTTGGCGAATATGGCTGCCATGTTTGCAATCTACCATGGTTC |
| | CCATGGGCTGGAGCATATTGCTAGGAGGGTACATAATGCCACTTT |
| | GATTTTGTCAGAAGGTCTCAAGCGAGCAGGGCATCAACTCCAGCA |
| | TGACCTGTTCTTTGATACCTTGAAGATTCAGTGTGGCTGCTCAGT |
| | GAAGGAGGTCTTGGGCAGGGCCGCTCAGCGGCAGATCAATTTTCG |
| | GCTTTTTGAGGATGGCACACTTGGTATTTCTCTTGATGAAACAGT |
| | CAATGAAAAAGATCTGGACGATTTGTTGTGGATCTTTGGTTGTGA |
| | GTCATCTGCAGAACTGGTTGCTGAAAGCATGGGAGAGGAGTGCAG |
| | AGGTATTCCAGGGTCTGTGTTCAAGAGGACCAGCCCGTTCCTCAC |
| | CCATCAAGTGTTCAACAGCTACCACTCTGAAACAAACATTGTCCG |
| | GTACATGAAGAAACTGGAAAATAAAGACATTTCCCTTGTTCACAG |
| | CATGATTCCACTGGGATCCTGCACCATGAAACTGAACAGTTCGTC |
| | TGAACTCGCACCTATCACATGGAAAGAATTTGCAAACATCCACCC |
| | CTTTGTGCCTCTGGATCAAGCTCAAGGATATCAGCAGCTTTTCCG |
| | AGAGCTTGAGAAGGATTTGTGTGAACTCACAGGTTATGACCAGGT |
| | CTGTTTCCAGCCAAACAGCGGAGCCCAGGGAGAATATGCTGGACT |
| | GGCCACTATCCGAGCCTACTTAAACCAGAAAGGAGAGGGGCACAG |
| | AACGGTTTGCCTCATTCCGAAATCAGCACATGGGACCAACCCAGC |
| | AAGTGCCCACATGGCAGGCATGAAGATTCAGCCTGTGGAGGTGGA |
| | TAAATATGGGAATATCGATGCAGTTCACCTCAAGGCCATGGTGGA |
| | TAAGCACAAGGAGAACCTAGCAGCTATCATGATTACATACCCATC |
| | CACCAATGGGGTGTTTGAAGAGAACATCAGTGACGTGTGTGACCT |
| | CATCCATCAACATGGAGGACAGGTCTACCTAGACGGGGCAAATAT |
| | GAATGCTCAGGTGGGAATCTGTCGCCCTGGAGACTTCGGGTCTGA |
| | TGTCTCGCACCTAAATCTTCACAAGACCTTCTGCATTCCCCACGG |
| | AGGAGGTGGTCCTGGCATGGGGCCCATCGGAGTGAAGAAACATCT |
| | CGCCCCGTTTTTGCCCAATCATCCCGTCATTTCACTAAAGCGGAA |
| | TGAGGATGCCTGTCCTGTGGGAACCGTCAGTGCGGCCCCATGGGG |
| | CTCCAGTTCCATCTTGCCCATTTCCTGGGCTTATATCAAGATGAT |
| | GGGAGGCAAGGGTCTTAAACAAGCCACGGAAACTGCGATATTAAA |
| | TGCCAACTACATGGCCAAGCGATTAGAAACACACTACAGAATTCT |
| | TTTCAGGGGTGCAAGAGGTTATGTGGGTCATGAATTTATTTTGGA |
| | CACGAGACCCTTCAAAAAGTCTGCAAATATTGAGGCTGTGGATGT |
| | GGCCAAGAGACTCCAGGATTATGGATTTCACGCCCCTACCATGTC |
| | CTGGCCTGTGGCAGGGACCCTCATGGTGGAGCCCACTGAGTCGGA |
| | GGACAAGGCAGAGCTGGACAGATTCTGTGATGCCATGATCAGCAT |
| | TCGGCAGGAAATTGCTGACATTGAGGAGGGCCGCATCGACCCCAG |
| | GGTCAATCCGCTGAAGATGTCTCCACACTCCCTGACCTGCGTTAC |
| | ATCTTCCCACTGGGACCGGCCTTATTCCAGAGAGGTGGCAGCATT |
| | CCCACTCCCCTTCGTGAAACCAGAGAACAAATTCTGGCCAACGAT |
| | TGCCCGGATTGATGACATATATGGAGATCAGCACCTGGTTTGTAC |
| | CTGCCCACCCATGGAAGTTTATGAGTCTCCATTTTCTGAACAAAA |
| | GAGGGCGTCTTCTTAGTCCTCTGTCCCTAAGTTTAAAGGACTGAT |
| | TTGATGCCTCTCCCCAGAGCATTTGATAAGCAAGAAAGATTTCAT |
| | CTCCCACCCCAGCCTCAAGTAGGAGTTTTATATACTGTGTATATC |
| | TCTGTAATCTCTGTCAAGGTAAATGTAAATACAGTAGCTGGAGGG |
| | AGTCGAAGCTGATGGTTGGAAGACGGATTTGCTTTGGTATTCTGC |
| | TTCCACATGTGCCAGTTGCCTGGATTGGGAGCCATTTTGTGTTTT |
| | GCGTAGAAAGTTTTAGGAACTTTAACTTTTAATGTGGCAAGTTTG |
| | CAGATGTCATAGAGGCTATCCTGGAGACTTAATAGACATTTTTTT |
| | GTTCCAAAAGAGTCCATGTGGACTGTGCCATCTGTGGGAAATCCC |
| | AGGGCAAATGTTTACATTTTGTATACCCTGAAGAACTCTTTTTCC |
| | TCTAATATGCCTAATCTGTAATCACATTTCTGAGTGTTCTCCTCT |
| | TTTTCTGTGTGAGGTTTTTTTTTTTTTAATCTGCATTTATTAGTA |
| | TTCTAATAAAAGCATCTTGATCGGAAGAAAAAAAAAAAAA |
| | (SEQ ID NO: 12) |
| Human MTHFD1 | MAPAEILNGKEISAQIRARLKNQVTQLKEQVPGFTPRLAILQVGN |
| Protein Sequence | RDDSNLYINVKLKAAEEIGIKATHIKLPRTTTESEVMKYITSLNE |
| C-1- | DSTVHGFLVQLPLDSENSINTEEVINAIAPEKDVDGLTSINAGKL |
| tetrahydrofolate | ARGDLNDCFIPCTPKGCLELIKETGVPIAGRHAVVVGRSKIVGAP |
| synthase, | MHDLLLWNNATVTTCHSKTAHLDEEVNKGDILVVATGQPEMVKGE |
| cytoplasmic | WIKPGAIVIDCGINYVPDDKKPNGRKVVGDVAYDEAKERASFITP |
| (NCBI Reference | VPGGVGPMTVAMLMQSTVESAKRFLEKFKPGKWMIQYNNLNLKTP |
| Sequence: | VPSDIDISRSCKPKPIGKLAREIGLLSEEVELYGETKAKVLLSAL |
| NP_005947.3) | ERLKHRPDGKYVVVTGITPTPLGEGKSTTTIGLVQALGAHLYQNV |
| | FACVRQPSQGPTFGIKGGAAGGGYSQVIPMEEFNLHLTGDIHAIT |
| | AANNLVAAAIDARIFHELTQTDKALFNRLVPSVNGVRRFSDIQIR |
| | RLKRLGIEKTDPTTLTDEEINRFARLDIDPETITWQRVLDTNDRF |
| | LRKITIGQAPTEKGHTRTAQFDISVASEIMAVLALTTSLEDMRER |
| | LGKMVVASSKKGEPVSAEDLGVSGALTVLMKDAIKPNLMQTLEGT |
| | PVFVHAGPFANIAHGNSSIIADRIALKLVGPEGFVVTEAGFGADI |
| | GMEKFFNIKCRYSGLCPHVVVLVATVRALKMHGGGPTVTAGLPLP |
| | KAYIQENLELVEKGFSNLKKQIENARMFGIPVVVAVNAFKTDTES |
| | ELDLISRLSREHGAFDAVKCTHWAEGGKGALALAQAVQRAAQAPS |
| | SFQLLYDLKLPVEDKIRIIAQKIYGADDIELLPEAQHKAEVYTKQ |
| | GFGNLPICMAKTHLSLSHNPEQKGVPTGFILPIRDIRASVGAGFL |
| | YPLVGTMSTMPGLPTRPCFYDIDLDPETEQVNGLF (SEQ ID |
| | NO: 13) |
| Human MTHFD1 | AATTACGGCCGGATTCCGGAGTCCTTTCCAGCTCCCTCTTCGGCC |
| mRNA Sequence | GGGTTTCCCGCCGAATACAAAGGCGCACTGTGAACTGGCTCTTTC |
| methylenetetra- | TTTCCGCCAATCATTTCCGCCAGCCATTCATCACCGATTTTCTTC |
| hydrofolate | ATCTTCCCCTCCCTCTTCCGTCCCGCAGTCCCCGACCTGTTAGCT |
| dehydrogenase | CTCGGTTAGTTAAGGGACTCGGGTCCTTCCGAACTGCGCATGCGC |
| (NADP+ dependent) | CACCGCGTCTGCAGGGGGAGAAGCGGGCAGGGGCGCAGGCGCAGT |
| 1, | AGTGTGATCCCCTGGCCAGTCCCTAAGCACGTGGGTTGGGTTGTC |
| methenyltetrahydro- | CTGCTTGGCTGCGGAGGGAGTGGAACCTCGATATTGGTGGTGTCC |
| folate | ATCGTGGGCAGCGGACTAATAAAGGCCATGGCGCCAGCAGAAATC |
| cyclohydrolase, | CTGAACGGGAAGGAGATCTCCGCGCAAATAAGGGCGAGACTGAAA |
| formyltetrahydro- | AATCAAGTCACTCAGTTGAAGGAGCAAGTACCTGGTTTCACACCA |
| folate synthetase | CGCCTGGCAATATTACAGGTTGGCAACAGAGATGATTCCAATCTT |
| (NCBI Reference | TATATAAATGTGAAGCTGAAGGCTGCTGAAGAGATTGGGATCAAA |
| Sequence: | GCCACTCACATTAAGTTACCAAGAACAACCACAGAATCTGAGGTG |
| NM_005956.3) | ATGAAGTACATTACATCTTTGAATGAAGACTCTACTGTACATGGG |
| | TTCTTAGTGCAGCTACCTTTAGATTCAGAGAATTCCATTAACACT |
| | GAAGAAGTGATCAATGCTATTGCACCCGAGAAGGATGTGGATGGA |
| | TTGACTAGCATCAATGCTGGGAAACTTGCTAGAGGTGACCTCAAT |
| | GACTGTTTCATTCCTTGTACGCCTAAGGGATGCTTGGAACTCATC |
| | AAAGAGACAGGGGTGCCGATTGCCGGAAGGCATGCTGTGGTGGTT |
| | GGGCGCAGTAAAATAGTTGGGGCCCCGATGCATGACTTGCTTCTG |
| | TGGAACAATGCCACAGTGACCACCTGCCACTCCAAGACTGCCCAT |
| | CTGGATGAGGAGGTAAATAAAGGTGACATCCTGGTGGTTGCAACT |
| | GGTCAGCCTGAAATGGTTAAAGGGGAGTGGATCAAACCTGGGGCA |
| | ATAGTCATCGACTGTGGAATCAATTATGTCCCAGATGATAAAAAA |
| | CCAAATGGGAGAAAAGTTGTGGGTGATGTGGCATACGACGAGGCC |
| | AAAGAGAGGGCGAGCTTCATCACTCCTGTTCCTGGCGGCGTAGGG |
| | CCCATGACAGTTGCAATGCTCATGCAGAGCACAGTAGAGAGTGCC |
| | AAGCGTTTCCTGGAGAAATTTAAGCCAGGAAAGTGGATGATTCAG |
| | TATAACAACCTTAACCTCAAGACACCTGTTCCAAGTGACATTGAT |
| | ATATCACGATCTTGTAAACCGAAGCCCATTGGTAAGCTGGCTCGA |
| | GAAATTGGTCTGCTGTCTGAAGAGGTAGAATTATATGGTGAAACA |
| | AAGGCCAAAGTTCTGCTGTCAGCACTAGAACGCCTGAAGCACCGG |
| | CCTGATGGGAAATACGTGGTGGTGACTGGAATAACTCCAACACCC |
| | CTGGGAGAAGGGAAAAGCACAACTACAATCGGGCTAGTGCAAGCC |
| | CTTGGTGCCCATCTCTACCAGAATGTCTTTGCGTGTGTGCGACAG |
| | CCTTCTCAGGGCCCCACCTTTGGAATAAAAGGTGGCGCTGCAGGA |
| | GGCGGCTACTCCCAGGTCATTCCTATGGAAGAGTTTAATCTCCAC |
| | CTCACAGGTGACATCCATGCCATCACTGCAGCTAATAACCTCGTT |
| | GCTGCGGCCATTGATGCTCGGATATTTCATGAACTGACCCAGACA |
| | GACAAGGCTCTCTTTAATCGTTTGGTGCCATCAGTAAATGGAGTG |
| | AGAAGGTTCTCTGACATCCAAATCCGAAGGTTAAAGAGACTAGGC |
| | ATTGAAAAGACTGACCCTACCACACTGACAGATGAAGAGATAAAC |
| | AGATTTGCAAGATTGGACATTGATCCAGAAACCATAACTTGGCAA |
| | AGAGTGTTGGATACCAATGATAGATTCCTGAGGAAGATCACGATT |
| | GGACAGGCTCCAACGGAGAAGGGTCACACACGGACGGCCCAGTTT |
| | GATATCTCTGTGGCCAGTGAAATTATGGCTGTCCTGGCTCTCACC |
| | ACTTCTCTAGAAGACATGAGAGAGAGACTGGGCAAAATGGTGGTG |
| | GCATCCAGTAAGAAAGGAGAGCCCGTCAGTGCCGAAGATCTGGGG |
| | GTGAGTGGTGCACTGACAGTGCTTATGAAGGACGCAATCAAGCCC |
| | AATCTCATGCAGACACTGGAGGGCACTCCAGTGTTTGTCCATGCT |
| | GGCCCGTTTGCCAACATCGCACATGGCAATTCCTCCATCATTGCA |
| | GACCGGATCGCACTCAAGCTTGTTGGCCCAGAAGGGTTTGTAGTG |
| | ACGGAAGCAGGATTTGGAGCAGACATTGGAATGGAAAAGTTTTTT |
| | AACATCAAATGCCGGTATTCCGGCCTCTGCCCCCACGTGGTGGTG |
| | CTTGTTGCCACTGTCAGGGCTCTCAAGATGCACGGGGGCGGCCCC |
| | ACGGTCACTGCTGGACTGCCTCTTCCCAAGGCTTACATACAGGAG |
| | AACCTGGAGCTGGTTGAAAAAGGCTTCAGTAACTTGAAGAAACAA |
| | ATTGAAAATGCCAGAATGTTTGGAATTCCAGTAGTAGTGGCCGTG |
| | AATGCATTCAAGACGGATACAGAGTCTGAGCTGGACCTCATCAGC |
| | CGCCTTTCCAGAGAACATGGGGCTTTTGATGCCGTGAAGTGCACT |
| | CACTGGGCAGAAGGGGGCAAGGGTGCCTTAGCCCTGGCTCAGGCC |
| | GTCCAGAGAGCAGCACAAGCACCCAGCAGCTTCCAGCTCCTTTAT |
| | GACCTCAAGCTCCCAGTTGAGGATAAAATCAGGATCATTGCACAG |
| | AAGATCTATGGAGCAGATGACATTGAATTACTTCCCGAAGCTCAA |
| | CACAAAGCTGAAGTCTACACGAAGCAGGGCTTTGGGAATCTCCCC |
| | ATCTGCATGGCTAAAACACACTTGTCTTTGTCTCACAACCCAGAG |
| | CAAAAAGGTGTCCCTACAGGCTTCATTCTGCCCATTCGCGACATC |
| | CGCGCCAGCGTTGGGGCTGGTTTTCTGTACCCCTTAGTAGGAACG |
| | ATGAGCACAATGCCTGGACTCCCCACCCGGCCCTGTTTTTATGAT |
| | ATTGATTTGGACCCTGAAACAGAACAGGTGAATGGATTATTCTAA |
| | ACAGATCACCATCCATCTTCAAGAAGCTACTTTGAAAGTCTGGCC |
| | AGTGTCTATTCAGGCCCACTGGGAGTTAGGAAGTATAAGTAAGCC |
| | AAGAGAAGTCAGCCCCTGCCCAGAAGATCTGAAACTAATAGTAGG |
| | AGTTTCCCCAGAAGTCATTTTCAGCCTTAATTCTCATCATGTATA |
| | AATTAACATAAATCATGCATGTCTGTTTACTTTAGTGACGTTCCA |
| | CAGAATAAAAGGAAACAAGTTTGCCATCAAAAAAAAAAAAAAAAA |
| | A (SEQ ID NO: 14) |
| Human MTHFD1L | MGTRLPLVLRQLRRPPQPPGPPRRLRVPCRASSGGGGGGGGGREG |
| Protein Sequence | LLGQRRPQDGQARSSCSPGGRTPAARDSIVREVIQNSKEVLSLLQ |
| monofunctional C1- | EKNPAFKPVLAIIQAGDDNLMQEINQNLAEEAGLNITHICLPPDS |
| tetrahydrofolate | SEAEIIDEILKINEDTRVHGLALQISENLFSNKVLNALKPEKDVD |
| synthase, | GVTDINLGKLVRGDAHECFVSPVAKAVIELLEKSVGVNLDGKKIL |
| mitochondrial | VVGAHGSLEAALQCLFQRKGSMTMSIQWKTRQLQSKLHEADIVVL |
| isoform | GSPKPEEIPLTWIQPGTTVLNCSHDFLSGKVGCGSPRIHFGGLIE |
| (NCBI Reference | EDDVILLAAALRIQNMVSSGRRWLREQQHRRWRLHCLKLQPLSPV |
| Sequence: | PSDIEISRGQTPKAVDVLAKEIGLLADEIEIYGKSKAKVRLSVLE |
| NP_001229696.1) | RLKDQADGKYVLVAGITPTPLGEGKSTVTIGLVQALTAHLNVNSF |
| | ACLRQPSQGPTFGVKGGAAGGGYAQVIPMEEFNLHLTGDIHAITA |
| | ANNLLAAAIDTRILHENTQTDKALYNRLVPLVNGVREFSEIQLAR |
| | LKKLGINKTDPSTLTEEEVSKFARLDIDPSTITWQRVLDTNDRFL |
| | RKITIGQGNTEKGHYRQAQFDIAVASEIMAVLALTDSLADMKARL |
| | GRMVVASDKSGQPVTADDLGVTGALTVLMKDAIKPNLMQTLEGTP |
| | VFVHAGPFANIAHGNSSVLADKIALKLVGEEGFVVTEAGFGADIG |
| | MEKFFNIKCRASGLVPNVVVLVATVRALKMHGGGPSVTAGVPLKK |
| | EYTEENIQLVADGCCNLQKQIQITQLFGVPVVVALNVFKTDTRAE |
| | IDLVCELAKRAGAFDAVPCYHWSVGGKGSVDLARAVREAASKRSR |
| | FQFLYDVQVPIVDKIRTIAQAVYGAKDIELSPEAQAKIDRYTQQG |
| | FGNLPICMAKTHLSLSHQPDKKGVPRDFILPISDVRASIGAGFIY |
| | PLVGTMSTMPGLPTRPCFYDIDLDTETEQVKGLF (SEQ ID |
| | NO: 15) |
| Human MTHFD1L | CCCCTAGGGGCCCCTGGGACGAGGAGGAAGCGCCAGGTCCTTCCC |
| mRNA Sequence | GCCGCCGCCGCCGCCGCCGCCGCCTGCTCCCCTGGCACGCGCCCC |
| methylenetetrahydr | GCCGCCCTCGGCAGCCGCAGCTCCGTGTCCCCTGAGAACCAGCCG |
| ofolate | TCCCGCGCCATGGGCACGCGTCTGCCGCTCGTCCTGCGCCAGCTC |
| dehydrogenase | CGCCGCCCGCCCCAGCCCCCGGGCCCTCCGCGCCGCCTCCGTGTG |
| (NADP+ dependent) | CCCTGTCGCGCTAGCAGCGGCGGCGGCGGAGGCGGCGGCGGTGGC |
| 1-like (MTHFD1L), | CGGGAGGGCCTGCTTGGACAGCGGCGGCCGCAGGATGGCCAGGCC |
| nuclear gene | CGGAGCAGCTGCAGCCCCGGCGGCCGAACGCCCGCGGCGCGGGAC |
| encoding | TCCATCGTCAGAGAAGTCATTCAGAATTCAAAAGAAGTTCTAAGT |
| mitochondrial | TTATTGCAAGAAAAAAACCCTGCCTTCAAGCCGGTTCTTGCAATT |
| protein, transcript | ATCCAGGCAGGTGACGACAACTTGATGCAGGAAATCAACCAGAAT |
| variant 1 | TTGGCTGAGGAGGCTGGTCTGAACATCACTCACATTTGCCTCCCT |
| (NCBI Reference | CCAGATAGCAGTGAAGCCGAGATTATAGATGAAATCTTAAAGATC |
| Sequence: | AATGAAGATACCAGAGTACATGGCCTTGCCCTTCAGATCTCTGAG |
| NM_001242767.1) | AACTTGTTTAGCAACAAAGTCCTCAATGCCTTGAAACCAGAAAAA |
| | GATGTGGATGGAGTAACAGACATAAACCTGGGGAAGCTGGTGCGA |
| | GGGGATGCCCATGAATGTTTTGTTTCACCTGTTGCCAAAGCTGTA |
| | ATTGAACTTCTTGAAAAATCAGTAGGTGTCAACCTAGATGGAAAG |
| | AAGATTTTGGTAGTGGGGGCCCATGGGTCTTTGGAAGCTGCTCTA |
| | CAATGCCTGTTCCAGAGAAAAGGGTCCATGACAATGAGCATCCAG |
| | TGGAAAACACGCCAGCTTCAAAGCAAGCTTCACGAGGCTGACATT |
| | GTGGTCCTAGGCTCACCTAAGCCAGAAGAGATTCCCCTTACTTGG |
| | ATACAACCAGGAACTACTGTTCTCAACTGCTCCCATGACTTCCTG |
| | TCAGGGAAGGTTGGGTGTGGCTCTCCAAGAATACATTTTGGTGGA |
| | CTCATTGAGGAAGATGATGTGATTCTCCTTGCTGCAGCTCTGCGA |
| | ATTCAGAACATGGTCAGTAGTGGAAGGAGATGGCTTCGTGAACAG |
| | CAGCACAGGCGGTGGAGACTTCACTGCTTGAAACTTCAGCCTCTC |
| | TCCCCTGTGCCAAGTGACATTGAGATTTCAAGAGGACAAACTCCA |
| | AAAGCTGTGGATGTCCTTGCCAAGGAGATTGGATTGCTTGCAGAT |
| | GAAATTGAAATCTATGGCAAAAGCAAAGCCAAAGTACGTTTGTCC |
| | GTGCTAGAAAGGTTAAAGGATCAAGCAGATGGAAAATACGTCTTA |
| | GTTGCTGGGATCACACCCACCCCTCTTGGAGAAGGGAAGAGCACA |
| | GTCACCATCGGGCTTGTGCAGGCTCTGACCGCACACCTGAATGTC |
| | AACTCCTTTGCCTGCTTGAGGCAGCCTTCCCAAGGACCGACGTTT |
| | GGAGTGAAAGGAGGAGCCGCGGGTGGTGGATATGCCCAGGTCATC |
| | CCCATGGAGGAGTTCAACCTTCACTTGACTGGAGACATCCACGCC |
| | ATCACCGCTGCCAATAACTTGCTGGCTGCCGCCATCGACACGAGG |
| | ATTCTTCATGAAAACACGCAAACAGATAAGGCTCTGTATAATCGG |
| | CTGGTTCCTTTAGTGAATGGTGTCAGAGAATTTTCAGAAATTCAG |
| | CTTGCTCGGCTAAAAAAACTGGGAATAAATAAGACTGATCCGAGC |
| | ACACTGACAGAAGAGGAAGTGAGTAAATTTGCCCGTCTCGACATC |
| | GACCCATCTACCATCACGTGGCAGAGAGTATTGGATACAAATGAC |
| | CGATTTCTACGAAAAATAACCATCGGGCAGGGAAACACAGAGAAG |
| | GGCCATTACCGGCAGGCGCAGTTTGACATCGCAGTGGCCAGCGAG |
| | ATCATGGCGGTGCTGGCCCTGACGGACAGCCTCGCAGACATGAAG |
| | GCACGGCTGGGAAGGATGGTGGTGGCCAGTGACAAAAGCGGGCAG |
| | CCTGTGACAGCAGATGATTTGGGGGTGACAGGTGCTTTGACAGTT |
| | TTGATGAAAGATGCAATAAAACCAAACCTGATGCAGACCCTGGAA |
| | GGGACACCTGTGTTCGTGCATGCGGGCCCTTTTGCTAACATTGCT |
| | CACGGCAACTCTTCAGTGTTGGCTGATAAAATTGCCCTGAAACTG |
| | GTTGGTGAAGAAGGATTTGTAGTGACCGAAGCTGGCTTTGGTGCT |
| | GACATCGGAATGGAGAAATTCTTCAACATCAAGTGCCGAGCTTCC |
| | GGCTTGGTGCCCAACGTGGTTGTGTTAGTGGCAACGGTGCGAGCT |
| | CTGAAGATGCATGGAGGCGGGCCAAGTGTAACGGCTGGTGTTCCT |
| | CTTAAGAAAGAATATACAGAGGAGAACATCCAGCTGGTGGCAGAC |
| | GGCTGCTGTAACCTCCAGAAGCAAATTCAGATCACTCAGCTCTTT |
| | GGGGTTCCCGTTGTGGTGGCTCTGAATGTCTTCAAGACCGACACC |
| | CGCGCTGAGATTGACTTGGTGTGTGAGCTTGCAAAGCGGGCTGGT |
| | GCCTTTGATGCAGTCCCCTGCTATCACTGGTCCGTTGGTGGAAAA |
| | GGATCGGTGGACTTGGCTCGGGCTGTGAGAGAGGCTGCGAGTAAA |
| | AGAAGCCGATTCCAGTTCCTGTATGATGTTCAGGTTCCAATTGTG |
| | GACAAGATAAGGACCATTGCTCAGGCTGTCTATGGAGCCAAAGAT |
| | ATTGAACTCTCTCCTGAGGCACAAGCCAAAATAGATCGTTACACT |
| | CAACAGGGTTTTGGAAATTTGCCCATCTGCATGGCAAAGACCCAC |
| | CTTTCTCTATCTCACCAACCTGACAAAAAAGGTGTGCCAAGGGAC |
| | TTCATCTTACCTATCAGTGACGTCCGGGCCAGCATAGGCGCTGGG |
| | TTCATTTACCCTTTGGTCGGAACGATGAGCACCATGCCAGGACTG |
| | CCCACCCGGCCCTGCTTTTATGACATAGATCTTGATACCGAAACA |
| | GAACAAGTTAAAGGCTTGTTCTAAGTGGACAAGGCTCTCACAGGA |
| | CCCGATGCAGACTCCTGAAACAGACTACTCTTTGCCTTTTTGCTG |
| | CAGTTGGAGAAGAAACTGAATTTGAAAAATGTCTGTTATGCAATG |
| | CTGGAGACATGGTGAAATAGGCCAAAGATTTCTTCTTCGTTCAAG |
| | ATGAATTCTGTTCACAGTGGAGTATGGTGTTCGGCAAAAGGACCT |
| | CCACCAAGACTGAAAGAAACTAATTTATTTCTGTTTCTGTGGAGT |
| | TTCCATTATTTCTACTGCTTACACTTTAGAATGTTTATTTTATGG |
| | GGACTAAGGGATTAGGAGTGTGAACTAAAAGGTAACATTTTCCAC |
| | TCTCAAGTTTTCTACTTTGTCTTTGAACTGAAAATAAACATGGAT |
| | CTAGAAAACCAAAAAAAAAAAAAAA (SEQ ID NO: 16) |
| Human MTHFD2 | MAATSLMSALAARLLQPAHSCSLRLRPFHLAAVRNEAVVISGRKL |
| Protein Sequence | AQQIKQEVRQEVEEWVASGNKRPHLSVILVGENPASHSYVLNKTR |
| bifunctional | AAAVVGINSETIMKPASISEEELLNLINKLNNDDNVDGLLVQLPL |
| methylenetetra- | PEHIDERRICNAVSPDKDVDGFHVINVGRMCLDQYSMLPATPWGV |
| hydrofolate | WEIIKRTGIPTLGKNVVVAGRSKNVGMPIAMLLHTDGAHERPGGD |
| dehydrogenase/ | ATVTISHRYTPKEQLKKHTILADIVISAAGIPNLITADMIKEGAA |
| cyclohydrolase, | VIDVGINRVHDPVTAKPKLVGDVDFEGVRQKAGYITPVPGGVGPM |
| mitochondrial | TVAMLMKNTIIAAKKVLRLEEREVLKSKELGVATN (SEQ ID |
| precursor | NO: 17) |
| (NCBI Reference | |
| Sequence: | |
| NP_006627.2) | |
| Human MTHFD2 | GGGGCCTGCCACGAGGCCGCAGTATAACCGCGTGGCCCGCGCGCG |
| mRNA Sequence | CGCTTCCCTCCCGGCGCAGTCACCGGCGCGGTCTATGGCTGCGAC |
| methylenetetra- | TTCTCTAATGTCTGCTTTGGCTGCCCGGCTGCTGCAGCCCGCGCA |
| hydrofolate | CAGCTGCTCCCTTCGCCTTCGCCCTTTCCACCTCGCGGCAGTTCG |
| dehydrogenase | AAATGAAGCTGTTGTCATTTCTGGAAGGAAACTGGCCCAGCAGAT |
| (NADP+ dependent) | CAAGCAGGAAGTGCGGCAGGAGGTAGAAGAGTGGGTGGCCTCAGG |
| 2, | CAACAAACGGCCACACCTGAGTGTGATCCTGGTTGGCGAGAATCC |
| methenyltetra- | TGCAAGTCACTCCTATGTCCTCAACAAAACCAGGGCAGCTGCAGT |
| hydrofolate | TGTGGGAATCAACAGTGAGACAATTATGAAACCAGCTTCAATTTC |
| cyclohydrolase | AGAGGAAGAATTGTTGAATTTAATCAATAAACTGAATAATGATGA |
| (MTHFD2), nuclear | TAATGTAGATGGCCTCCTTGTTCAGTTGCCTCTTCCAGAGCATAT |
| gene encoding | TGATGAGAGAAGGATCTGCAATGCTGTTTCTCCAGACAAGGATGT |
| mitochondrial | TGATGGCTTTCATGTAATTAATGTAGGACGAATGTGTTTGGATCA |
| protein, transcript | GTATTCCATGTTACCGGCTACTCCATGGGGTGTGTGGGAAATAAT |
| variant 1 | CAAGCGAACTGGCATTCCAACCCTAGGGAAGAATGTGGTTGTGGC |
| (NCBI Reference | TGGAAGGTCAAAAAACGTTGGAATGCCCATTGCAATGTTACTGCA |
| Sequence: | CACAGATGGGGCGCATGAACGTCCCGGAGGTGATGCCACTGTTAC |
| NM_006636.3) | AATATCTCATCGATATACTCCCAAAGAGCAGTTGAAGAAACATAC |
| | AATTCTTGCAGATATTGTAATATCTGCTGCAGGTATTCCAAATCT |
| | GATCACAGCAGATATGATCAAGGAAGGAGCAGCAGTCATTGATGT |
| | GGGAATAAATAGAGTTCACGATCCTGTAACTGCCAAACCCAAGTT |
| | GGTTGGAGATGTGGATTTTGAAGGAGTCAGACAAAAAGCTGGGTA |
| | TATCACTCCAGTTCCTGGAGGTGTTGGCCCCATGACAGTGGCAAT |
| | GCTAATGAAGAATACCATTATTGCTGCAAAAAAGGTGCTGAGGCT |
| | TGAAGAGCGAGAAGTGCTGAAGTCTAAAGAGCTTGGGGTAGCCAC |
| | TAATTAACTACTGTGTCTTCTGTGTCACAAACAGCACTCCAGGCC |
| | AGCTCAAGAAGCAAAGCAGGCCAATAGAAATGCAATATTTTTAAT |
| | TTATTCTACTGAAATGGTTTAAAATGATGCCTTGTATTTATTGAA |
| | AGCTTAAATGGGTGGGTGTTTCTGCACATACCTCTGCAGTACCTC |
| | ACCAGGGAGCATTCCAGTATCATGCAGGGTCCTGTGATCTAGCCA |
| | GGAGCAGCCATTAACCTAGTGATTAATATGGGAGACATTACCATA |
| | TGGAGGATGGATGCTTCACTTTGTCAAGCACCTCAGTTACACATT |
| | CGCCTTTTCTAGGATTGCATTTCCCAAGTGCTATTGCAATAACAG |
| | TTGATACTCATTTTAGGTACCAAACCTTTTGAGTTCAACTGATCA |
| | AACCAAAGGAAAAGTGTTGCTAGAGAAAATTAGGGAAAAGGTGAA |
| | AAAGAAAAAATGGTAGTAATTGAGCAGAAAAAAATTAATTTATAT |
| | ATGTATTGATTGGCAACCAGATTTATCTAAGTAGAACTGAATTGG |
| | CTAGGAAAAAAGAAAAACTGCATGTTAATCATTTTCCTAAGCTGT |
| | CCTTTTGAGGCTTAGTCAGTTTATTGGGAAAATGTTTAGGATTAT |
| | TCCTTGCTATTAGTACTCATTTTATGTATGTTACCCTTCAGTAAG |
| | TTCTCCCCATTTTAGTTTTCTAGGACTGAAAGGATTCTTTTCTAC |
| | ATTATACATGTGTGTTGTCATATTTGGCTTTTGCTATATACTTTA |
| | ACTTCATTGTTAAATTTTTGTATTGTATAGTTTCTTTGGTGTATC |
| | TTAAAACCTATTTTTGAAAAACAAACTTGGCTTGATAATCATTTG |
| | GGCAGCTTGGGTAAGTACGCAACTTACTTTTCCACCAAAGAACTG |
| | TCAGCAGCTGCCTGCTTTTCTGTGATGTATGTATCCTGTTGACTT |
| | TTCCAGAAATTTTTTAAGAGTTTGAGTTACTATTGAATTTAATCA |
| | GACTTTCTGATTAAAGGGTTTTCTTTCTTTTTTAATAAAACACAT |
| | CTGTCTGGTATGGTATGAATTTCTGAAAAAAAAAAAAAAAAAAAA |
| | AAA (SEQ ID NO: 18) |
| Human MTHFD2L | MTVPVRGFSLLRGRLGRAPALGRSTAPSVRAPGEPGSAFRGFRSS |
| Protein Sequence | GVRHEAIIISGTEMAKHIQKEIQRGVESWVSLGNRRPHLSIILVG |
| probable | DNPASHTYVRNKIRAASAVGICSELILKPKDVSQEELLDVTDQLN |
| bifunctional | MDPRVSGILVQLPLPDHVDERTICNGIAPEKDVDGFHIINIGRLC |
| methylenetetra- | LDQHSLIPATASAVWEIIKRTGIQTFGKNVVVAGRSKNVGMPIAM |
| hydrofolate | LLHTDGEHERPGGDATVTIAHRYTPKEQLKIHTQLADIIIVAAGI |
| dehydrogenase/ | PKLITSDMVKEGAAVIDVGINYVHDPVTGKTKLVGDVDFEAVKKK |
| cyclohydrolase 2 | AGFITPVPGGVGPMTVAMLLKNTLLAAKKIIY (SEQ ID |
| (NCBI Reference | NO: 19) |
| Sequence: | |
| NP_001138450.1) | |
| Human MTHFD2L | CAGTCCGGAAGCCGGGGATCCGCGGCCATGACGGTGCCGGTCCGC |
| mRNA Sequence | GGCTTCTCGCTGCTCCGCGGCCGCCTTGGCCGAGCGCCGGCGTTG |
| methylenetetra- | GGCAGAAGCACAGCACCCTCCGTAAGGGCACCGGGAGAGCCCGGG |
| hydrofolate | AGTGCGTTCCGGGGCTTTCGGAGCAGCGGTGTGAGACATGAAGCC |
| dehydrogenase | ATTATTATATCAGGAACCGAAATGGCCAAGCATATCCAGAAAGAA |
| (NADP+ dependent) | ATACAGCGAGGTGTGGAATCATGGGTTTCCCTTGGAAACAGAAGA |
| 2-like | CCTCACCTCAGTATAATTTTAGTGGGAGATAACCCAGCAAGCCAT |
| (NCBI Reference | ACATATGTCAGGAATAAGATAAGAGCTGCCTCTGCTGTAGGTATT |
| Sequence: | TGTAGTGAGCTCATTCTAAAACCTAAGGATGTTTCTCAGGAAGAA |
| NM_001144978.1) | CTTTTGGACGTAACTGATCAATTGAATATGGACCCAAGAGTCAGC |
| | GGTATATTAGTTCAGTTACCACTACCAGACCACGTTGATGAGCGA |
| | ACAATATGCAATGGAATTGCCCCAGAAAAAGATGTAGATGGATTT |
| | CATATTATCAATATTGGAAGATTGTGCCTTGATCAGCATTCTCTC |
| | ATACCTGCCACTGCCAGTGCTGTTTGGGAAATAATAAAAAGAACA |
| | GGAATTCAAACATTTGGAAAAAATGTGGTTGTGGCTGGAAGATCC |
| | AAGAACGTAGGGATGCCTATTGCCATGCTTTTACACACTGATGGA |
| | GAGCATGAACGGCCAGGAGGTGATGCAACTGTGACAATAGCTCAC |
| | AGATACACCCCCAAAGAGCAACTGAAGATTCATACGCAGCTGGCA |
| | GATATTATCATAGTTGCTGCAGGTATTCCAAAGTTGATTACGTCT |
| | GATATGGTTAAAGAAGGTGCTGCTGTAATTGATGTGGGTATCAAC |
| | TATGTCCACGATCCAGTGACAGGAAAGACAAAATTAGTTGGAGAT |
| | GTGGACTTCGAAGCTGTTAAAAAGAAAGCTGGCTTTATCACTCCA |
| | GTTCCAGGAGGTGTGGGACCCATGACAGTGGCAATGCTTCTGAAG |
| | AACACCCTTCTGGCAGCTAAAAAAATCATTTACTAGATCACATGA |
| | AAGGATAAAGCAAACTGAAGTCATGCTATTTGTTTATTTGACAAA |
| | GGGTAAAACCTTTATATTTTACTACAAAGCTATTTATTTCTACAT |
| | GGTATTTATTTTTTCATGGGTGAAATCATTGTGAATCAATTGATT |
| | CACATAGTTTTATGCATTTCCTGCTAATTTATTTTGAGTTTTAAG |
| | AAAACAACCAAAACAATTCCAATGAAAATTTTAGTAACAATTGTT |
| | TATTTTGAGGGTATTTGTTCATAACATTAAAACAATAAAGGGCTC |
| | ATAATAAATAAATATATTTTTGACACAATTAAATATTACATAGAG |
| | TATGTTTACAACAAATATCCTGTCAGCCAAATGGTTACCCATATA |
| | AAATGTAATTTAGGTTTTGCTACTTGCATGCTAACATTTTTAATG |
| | TATTTTATGATCTATGTCATATATTAAAAAAGAGCTTGCTTACTA |
| | CAAGAAAAATATTGAAATATTGAAAATATTGAAAATATTATTATT |
| | GAAAATAAAATCTTGATCTCAACTATCCCCCAAATGCATCCTATA |
| | AGTCCATCCTAATGAGAAATGATGTTCTATTTAAGGAAAGGAAAA |
| | TATTCCGGGAAGGCAAAAAATGCAGTGCTGTTTGGAAGTGTAATG |
| | ATTTTATCACATGGTGAATGACTACTAAGAGTAATGATTATATCA |
| | CATTGTGAATGACTACTTGCCACAGTAAAAATACATGAAGAATGT |
| | GTTAGGTTTAAACGTCGTTTCTTTCTTCTAAAAAATATTTGGTTA |
| | GTACCTTCACTGAGCAATAGTGGAAAAATAAAAAAATAAGTAAAC |
| | AGAAAAAACTAAAGTTGTATTTTCCCACAAATATAGTATGAATGA |
| | GGTCATATTAAAGAACAGCAACTGTTAATGTTTGTTCACAAATTC |
| | AGAAATCTAATAGGAAAACATGATACTTTCAATGTGCCAAAACTA |
| | AACCTTAGTATACAACTAAAAATCTCCTGCCTTCTTGCCTACCTG |
| | TCTTCCCTCTTCTGTTACAGAATTTGTTCCTCAAAGTAGATGCAA |
| | TGTTTCTAACACAATTTAAATTAGGAAATATATATGAATGTCGTT |
| | GAAGTCTATTTTGAGACTGCTAAAGCTATTAATTGATACTGTGTT |
| | TTTATGCCCAAATCCCAGTATGTTTATGTACCAATAATGACTCTT |
| | ACCCAGCGCATGTCTTTATCAGTGTGTACTCGTGACGATTTGTGT |
| | GAAAATAGACTTGATGTTTATAATTAATACCATTACAACTGTATA |
| | ATAAAAGCAATTTGAAGAAAAAAAAAAAAAAA (SEQ ID |
| | NO: 20) |
| Human MTHFS | MAAAAVSSAKRSLRGELKQRLRAMSAEERLRQSRVLSQKVIAHSE |
| Protein Sequence | YQKSKRISIFLSMQDEIETEEIIKDIFQRGKICFIPRYRFQSNHM |
| 5- | DMVRIESPEEISLLPKTSWNIPQPGEGDVREEALSTGGLDLIFMP |
| formyltetrahydro- | GLGFDKHGNRLGRGKGYYDAYLKRCLQHQEVKPYTLALAFKEQIC |
| folate cyclo-ligase | LQVPVNENDMKVDEVLYEDSSTA (SEQ ID NO: 21) |
| isoform a | |
| (NCBI Reference | |
| Sequence: | |
| NP_006432.1) | |
| Human MTHFS | AGACCGAACCCGAGGGCGCCCAGGGCGCCGAGGGCGGGACTGGAC |
| mRNA Sequence | TCGGCTTGGGCGTGAGATGGCGGCGGCAGCGGTGAGCAGCGCCAA |
| 5,10- | GCGGAGCCTGCGGGGAGAGCTGAAGCAGCGTCTGCGGGCGATGAG |
| methenyltetrahydro- | TGCCGAGGAGCGGCTACGCCAGTCCCGCGTACTGAGCCAGAAGGT |
| folate synthetase | GATTGCCCACAGTGAGTATCAAAAGTCCAAAAGAATTTCCATCTT |
| (5- | TCTGAGCATGCAAGATGAAATTGAGACAGAAGAGATCATCAAGGA |
| formyltetrahydro- | CATTTTCCAACGAGGCAAAATCTGCTTCATCCCTCGGTACCGGTT |
| folate cyclo-ligase) | CCAGAGCAATCACATGGATATGGTGAGAATAGAATCACCAGAGGA |
| (NCBI Reference | AATTTCTTTACTTCCCAAAACATCCTGGAATATCCCTCAGCCTGG |
| Sequence: | TGAGGGTGATGTTCGGGAGGAGGCCTTGTCCACAGGGGGACTTGA |
| NM_006441.3) | TCTCATCTTCATGCCAGGCCTTGGGTTTGACAAACATGGCAACCG |
| | ACTGGGGAGGGGCAAGGGCTACTATGATGCCTATCTGAAGCGCTG |
| | TTTGCAGCATCAGGAAGTGAAGCCCTACACCCTGGCGTTGGCTTT |
| | CAAAGAACAGATTTGCCTCCAGGTCCCAGTGAATGAAAACGACAT |
| | GAAGGTAGATGAAGTCCTTTACGAAGACTCGTCAACAGCTTAAAT |
| | CTGGATTACTACAGCCAAATAATCAGTGTTTTATATGAGAGTAAA |
| | GCAAAGTATGTGTATTTTTCCCTTGTCAAAAATTAGTTGAAATTG |
| | TTCATTAATGTGAATACAGACTGCATTTTAAAATTGTAATTATGA |
| | AATACCTTATATAAAACCATCTTTAAAAACCAATAGAAGTGTGAA |
| | TAGTAGAATATTAATTAAAATGGAGGCTATCAGCCTGTGATTTTC |
| | AGCTTAACTTCCTGGTGTTAATGTGACAAGTTGATCTGTCTACTT |
| | TGCAATTTAAGTTAAATATTTATGAGGAACTGTGCTCCGACTGAG |
| | TGCGAGAGGAAGGTAAACTTGCGGGAGTGGGCACTGTATTTCATT |
| | ACGCCTTTCATGACCTGGTCTGTCCTGGCAGGCACATGGAGACTT |
| | GGGGACTATTAATTTATTTGTTGGTATTTGCTTTGGATACAGAAT |
| | TCCCTAAGAATATTATCTCACTTCATCATGACTTCCTTTACCCAC |
| | TCTAGAATTTTATGTTGGACACTTTGTAGCTTTTGGTGGTTAGTG |
| | GAGGGAAACCTTTTATGATTTAAATACTTTTACTCCACTGATTGG |
| | TTACCATCACTGCATGTATCAGCCCTTGATGAGTTTAAGATCTAG |
| | TATCTTATAAGTTAGAAATTATTTCTGTTTACTCATGGTTTCTGC |
| | TTTGGAAATGAAATTTGCTGTGAGTTGAAAGTTGGCAGATGGCAA |
| | CACAGCTAGGGAGCAATAATTTTGTTGTGGGGAGGATTTGGTCAT |
| | CTCCAGAAACCCGGGAGTCACGTGGCTCTCTTACTCACACTCCAT |
| | GTCCATCCTGTCACCAGGTCTTGTTGATTTTGCCTCTGAAATGCC |
| | TCTTAAATCTATTCTCTCCTTTCAATTTTCCTGTCACTCCTTTTG |
| | TCCAGCCTTTTAGCATTTCTAAGCTGGACTAGGCGAGAGTGTCAC |
| | ACCTGCTTCCCTTGGCTTCCATCTTGCCTCTTCCAGTTTATTTCT |
| | CAAGCTGCAGTCAAACTGATCTTAGAAAACACGAATCTAATCATG |
| | CAGCACCCCTGACTAAGGTCTTCCGGTGGCTGTTCAGAGCCTCTT |
| | GGGTAGCAAACAGATGGCTTCTGTTGTATACAAAGCCCCTTAAGA |
| | GAGGTCTCCTCATCTACTTTTTCTAGCCTCTTCTCTCCCAACTCT |
| | GTATTCTCCTGTAACACTGACTGAGCACTGCAGGCTTCTGCCCTT |
| | TGCACATAGTAAGCATGCATTTCTCTCTGTCTGAAATGCTCTTTC |
| | TGTTGTTCATCTAGAAGACTGTTTTCCCTTGAAGACTCAGCCCTA |
| | GCATCACCTCTTCTGTGAAGTCTTCTGCTACTTTCCCAAGCAGAG |
| | TGAGTGTTCCTTCCCTTGTCTGAGTGGCCTTGGCCATTGATGTGC |
| | TTATCATGTTGTCTTACGTATCAAATTATTTGTTGTCATATCTGT |
| | CCCCTTCACCATACTGTGAGCTCCAAAGAAAAGAAGTGATCTTTA |
| | TACTTCTTATGCTTAGTACACAACTAGACATATAGTGTGTGTGTG |
| | AGAGAGAGAATTTTTTAATGAAATAATTGAATACATTGGAAGTGT |
| | TTCATTCAAAATACTCATCCATTATTCTTTGGATAGTAGCATAAA |
| | TTTGATGTTTTATGTACAAAAGTAAAAACATTTGAAAAATAAAAA |
| | AAAAAA (SEQ ID NO: 22) |
| Human MTRR | MRRFLLLYATQQGQAKAIAEEICEQAVVHGFSADLHCISESDKYD |
| Protein Sequence | LKTETAPLVVVVSTTGTGDPPDTARKFVKEIQNQTLPVDFFAHLR |
| methionine synthase | YGLLGLGDSEYTYFCNGGKIIDKRLQELGARHFYDTGHADDCVGL |
| reductase isoform 1 | ELVVEPWIAGLWPALRKHFRSSRGQEEISGALPVASPASSRTDLV |
| (NCBI Reference | KSELLHIESQVELLRFDDSGRKDSEVLKQNAVNSNQSNVVIEDFE |
| Sequence: | SSLTRSVPPLSQASLNIPGLPPEYLQVHLQESLGQEESQVSVTSA |
| NP_002445.2) | DPVFQVPISKAVQLTTNDAIKTTLLVELDISNTDFSYQPGDAFSV |
| | ICPNSDSEVQSLLQRLQLEDKREHCVLLKIKADTKKKGATLPQHI |
| | PAGCSLQFIFTWCLEIRAIPKKAFLRALVDYTSDSAEKRRLQELC |
| | SKQGAADYSRFVRDACACLLDLLLAFPSCQPPLSLLLEHLPKLQP |
| | RPYSCASSSLFHPGKLHFVFNIVEFLSTATTEVLRKGVCTGWLAL |
| | LVASVLQPNIHASHEDSGKALAPKISISPRTTNSFHLPDDPSIPI |
| | IMVGPGTGIAPFIGFLQHREKLQEQHPDGNFGAMWLFFGCRHKDR |
| | DYLFRKELRHFLKHGILTHLKVSFSRDAPVGEEEAPAKYVQDNIQ |
| | LHGQQVARILLQENGHIYVCGDAKNMAKDVHDALVQIISKEVGVE |
| | KLEAMKTLATLKEEKRYLQDIWS (SEQ ID NO: 23) |
| Human MTRR | GGAGCTTTCTATTGGTCCTGGGTACCGAGCATGGGCGCTGCGTCA |
| mRNA Sequence | GTGCGCGCTGGCGCAAGGTTGGTGGAAGTCGCGTTGTGCAGGTTC |
| 5- | GTGCCCGGCTGGCGCGGCGTGGTTTCACTGTTACATGCCTTGAAG |
| methyltetrahydro- | TGATGAGGAGGTTTCTGTTACTATATGCTACACAGCAGGGACAGG |
| folate-homocysteine | CAAAGGCCATCGCAGAAGAAATATGTGAGCAAGCTGTGGTACATG |
| methyltransferase | GATTTTCTGCAGATCTTCACTGTATTAGTGAATCCGATAAGTATG |
| reductase (MTRR), | ACCTAAAAACCGAAACAGCTCCTCTTGTTGTTGTGGTTTCTACCA |
| transcript variant 1 | CGGGCACCGGAGACCCACCCGACACAGCCCGCAAGTTTGTTAAGG |
| (NCBI Reference | AAATACAGAACCAAACACTGCCGGTTGATTTCTTTGCTCACCTGC |
| Sequence: | GGTATGGGTTACTGGGTCTCGGTGATTCAGAATACACCTACTTTT |
| NM_002454.2) | GCAATGGGGGGAAGATAATTGATAAACGACTTCAAGAGCTTGGAG |
| | CCCGGCATTTCTATGACACTGGACATGCAGATGACTGTGTAGGTT |
| | TAGAACTTGTGGTTGAGCCGTGGATTGCTGGACTCTGGCCAGCCC |
| | TCAGAAAGCATTTTAGGTCAAGCAGAGGACAAGAGGAGATAAGTG |
| | GCGCACTCCCGGTGGCATCACCTGCATCCTCGAGGACAGACCTTG |
| | TGAAGTCAGAGCTGCTACACATTGAATCTCAAGTCGAGCTTCTGA |
| | GATTCGATGATTCAGGAAGAAAGGATTCTGAGGTTTTGAAGCAAA |
| | ATGCAGTGAACAGCAACCAATCCAATGTTGTAATTGAAGACTTTG |
| | AGTCCTCACTTACCCGTTCGGTACCCCCACTCTCACAAGCCTCTC |
| | TGAATATTCCTGGTTTACCCCCAGAATATTTACAGGTACATCTGC |
| | AGGAGTCTCTTGGCCAGGAGGAAAGCCAAGTATCTGTGACTTCAG |
| | CAGATCCAGTTTTTCAAGTGCCAATTTCAAAGGCAGTTCAACTTA |
| | CTACGAATGATGCCATAAAAACCACTCTGCTGGTAGAATTGGACA |
| | TTTCAAATACAGACTTTTCCTATCAGCCTGGAGATGCCTTCAGCG |
| | TGATCTGCCCTAACAGTGATTCTGAGGTACAAAGCCTACTCCAAA |
| | GACTGCAGCTTGAAGATAAAAGAGAGCACTGCGTCCTTTTGAAAA |
| | TAAAGGCAGACACAAAGAAGAAAGGAGCTACCTTACCCCAGCATA |
| | TACCTGCGGGATGTTCTCTCCAGTTCATTTTTACCTGGTGTCTTG |
| | AAATCCGAGCAATTCCTAAAAAGGCATTTTTGCGAGCCCTTGTGG |
| | ACTATACCAGTGACAGTGCTGAAAAGCGCAGGCTACAGGAGCTGT |
| | GCAGTAAACAAGGGGCAGCCGATTATAGCCGCTTTGTACGAGATG |
| | CCTGTGCCTGCTTGTTGGATCTCCTCCTCGCTTTCCCTTCTTGCC |
| | AGCCACCACTCAGTCTCCTGCTCGAACATCTTCCTAAACTTCAAC |
| | CCAGACCATATTCGTGTGCAAGCTCAAGTTTATTTCACCCAGGAA |
| | AGCTCCATTTTGTCTTCAACATTGTGGAATTTCTGTCTACTGCCA |
| | CAACAGAGGTTCTGCGGAAGGGAGTATGTACAGGCTGGCTGGCCT |
| | TGTTGGTTGCTTCAGTTCTTCAGCCAAACATACATGCATCCCATG |
| | AAGACAGCGGGAAAGCCCTGGCTCCTAAGATATCCATCTCTCCTC |
| | GAACAACAAATTCTTTCCACTTACCAGATGACCCCTCAATCCCCA |
| | TCATAATGGTGGGTCCAGGAACCGGCATAGCCCCGTTTATTGGGT |
| | TCCTACAACATAGAGAGAAACTCCAAGAACAACACCCAGATGGAA |
| | ATTTTGGAGCAATGTGGTTGTTTTTTGGCTGCAGGCATAAGGATA |
| | GGGATTATCTATTCAGAAAAGAGCTCAGACATTTCCTTAAGCATG |
| | GGATCTTAACTCATCTAAAGGTTTCCTTCTCAAGAGATGCTCCTG |
| | TTGGGGAGGAGGAAGCCCCAGCAAAGTATGTGCAAGACAACATCC |
| | AGCTTCATGGCCAGCAGGTGGCGAGAATCCTCCTCCAGGAGAACG |
| | GCCATATTTATGTGTGTGGAGATGCAAAGAATATGGCCAAGGATG |
| | TACATGATGCCCTTGTGCAAATAATAAGCAAAGAGGTTGGAGTTG |
| | AAAAACTAGAAGCAATGAAAACCCTGGCCACTTTAAAAGAAGAAA |
| | AACGCTACCTTCAGGATATTTGGTCATAAAACCAGAAATTAAAGA |
| | AAGAGGATTAAGCTTTTTTGACTGAAAGTACTAAAAGTCAGCTTT |
| | ACTAGTGCCAAACCTTTAAATTTTCAAAAGAAAATTTTCTTTCAA |
| | CATTTCTTGAAGGACATGGAGTGGAGATTGGATCATTTAACAATA |
| | TAACAAAACTTCCTGATTTGATTTTACGTATCTTCTATCTACGCC |
| | CTTCCTGTGCCTGTGACTCTCCCCAAATTGCCCTGTTGCCTTGAG |
| | CTCTTCTGAGCTAAGGCAGCCTTCAGTCCCTATCAGCGCCTCCTT |
| | TACTTCCCAGAGAACTTCACAGAGACTCTGTCCTTCCATGCAAAG |
| | GCTTCCTGAAATAGGGAGACTGACTGAGTAGCTCATTCTTGTGAC |
| | TTACAGTGCCAACATTTAAAAAAGTATGAAAATGATTTATTTTTA |
| | TATGATGTATACCCATAAAGAATGCTCATATTAATGTACTTAAAT |
| | TACACATGTAGAGCATATCTGTTATATGTTTATGTAACTATCAAA |
| | TGGTTATTTGTTACTAAAGCTATATTTCTGATAAAAAATATTTTA |
| | GGATAATTGCCTACAGAGGGATTTATTTTTATGATGCTGGAAATA |
| | TGAAATGTATTTTAAAATTTCACTCTGGCATATGATTTATCTATC |
| | ACCATTACTTTTTTTTAAGTCACAATTTCAGAATTTTGGGACATT |
| | TGCATTCAATTTACAGGTACCAGTACGTACATATTTTAATAGAAA |
| | GATACAACCTTTTTATTTTCACTCCTTTTATTTCTGCTGCTTGGC |
| | ACATTTTTGAGTTTTCCCACATTATTTGTCTCCATGATACCACTC |
| | AAGCAGTGTGCTGGACCTAAAATACTGACTTTAGTTAGTATCCTT |
| | GGATTTTTAGATTCCCAGTGTCTAATTCCCTGTTATAATTTGCAC |
| | AAACAAAACAAAATGTTATGATAATCTTTCTCCACTGTTCTAATA |
| | TATATTGTATTTTTATTTGATAGCTTGGGATTTAAAACATCTCTG |
| | TTGAAGGCTTTTGATCCTTTTGAGAAATAAAGATCTGAAAGAAAT |
| | GGCATAATCTTAAAAAAAAAAAAAAAAAAAAA (SEQ ID |
| | NO: 24) |
| Human SHMT1 | MTMPVNGAHKDADLWSSHDKMLAQPLKDSDVEVYNIIKKESNRQR |
| Protein Sequence | VGLELIASENFASRAVLEALGSCLNNKYSEGYPGQRYYGGTEFID |
| serine | ELETLCQKRALQAYKLDPQCWGVNVQPYSGSPANFAVYTALVEPH |
| hydroxymethyl- | GRIMGLDLPDGGHLTHGFMTDKKKISATSIFFESMPYKVNPDTGY |
| transferase, | INYDQLEENARLFHPKLIIAGTSCYSRNLEYARLRKIADENGAYL |
| cytosolic isoform 1 | MADMAHISGLVAAGVVPSPFEHCHVVTTTTHKTLRGCRAGMIFYR |
| (NCBI Reference | KGVKSVDPKTGKEILYNLESLINSAVFPGLQGGPHNHAIAGVAVA |
| Sequence: | LKQAMTLEFKVYQHQVVANCRALSEALTELGYKIVTGGSDNHLIL |
| NP_004160.3) | VDLRSKGTDGGRAEKVLEACSIACNKNTCPGDRSALRPSGLRLGT |
| | PALTSRGLLEKDFQKVAHFIHRGIELTLQIQSDTGVRATLKEFKE |
| | RLAGDKYQAAVQALREEVESFASLFPLPGLPDF (SEQ ID |
| | NO: 25) |
| Human SHMT1 | GCCTGGCGCGCAGAGTGCACCTTCCTGAGCTCGAGCGGTCCAGCG |
| mRNA Sequence | CCAAGTTCGGGGTTTGGGGTTGGAGCGGCTGGTCACGTGGCTGGC |
| serine | CCGCGGCGGTGCGCGGGGCGTTGGGTCAGCGGGTCTGGGACTGGT |
| hydroxymethyltrans- | GGCACCGGCGGCGGCGTAGGACGGAGGCGTCGCTAGGCAGCTTCG |
| ferase 1 (soluble) | AACCAGTGCAATGACGATGCCAGTCAACGGGGCCCACAAGGATGC |
| (SHMT1), nuclear | TGACCTGTGGTCCTCACATGACAAGATGCTGGCACAACCCCTCAA |
| gene encoding | AGACAGTGATGTTGAGGTTTACAACATCATTAAGAAGGAGAGTAA |
| mitochondrial | CCGGCAGAGGGTTGGATTGGAGCTGATTGCCTCGGAGAATTTCGC |
| protein, transcript | CAGCCGAGCAGTTTTGGAGGCCCTAGGCTCTTGCTTAAATAACAA |
| variant 1 | ATACTCTGAGGGGTACCCGGGCCAGAGATACTATGGCGGGACTGA |
| (NCBI Reference | GTTTATTGATGAACTGGAGACCCTCTGTCAGAAGCGAGCCCTGCA |
| Sequence: | GGCCTATAAGCTGGACCCACAGTGCTGGGGGGTCAACGTCCAGCC |
| NM_004169.3) | CTACTCAGGCTCCCCTGCAAACTTTGCTGTGTACACTGCCCTGGT |
| | GGAACCCCATGGGCGCATCATGGGCCTGGACCTTCCGGATGGGGG |
| | CCACCTGACCCATGGGTTCATGACAGACAAGAAGAAAATCTCTGC |
| | CACGTCCATCTTCTTTGAATCTATGCCCTACAAGGTGAACCCAGA |
| | TACTGGCTACATCAACTATGACCAGCTGGAGGAGAACGCACGCCT |
| | CTTCCACCCGAAGCTGATCATCGCAGGAACCAGCTGCTACTCCCG |
| | AAACCTGGAATATGCCCGGCTACGGAAGATTGCAGATGAGAACGG |
| | GGCGTATCTCATGGCGGACATGGCTCACATCAGCGGGCTGGTGGC |
| | GGCTGGCGTGGTGCCCTCCCCATTTGAACACTGCCATGTGGTGAC |
| | CACCACCACTCACAAGACCCTGCGAGGCTGCCGAGCTGGCATGAT |
| | CTTCTACAGGAAAGGAGTGAAAAGTGTGGATCCCAAGACTGGCAA |
| | AGAGATTCTGTACAACCTGGAGTCTCTTATCAATTCTGCTGTGTT |
| | CCCTGGCCTGCAGGGAGGTCCCCACAACCACGCCATTGCTGGGGT |
| | TGCTGTGGCACTGAAGCAAGCTATGACTCTGGAATTTAAAGTTTA |
| | TCAACACCAGGTGGTGGCCAACTGCAGGGCTCTGTCTGAGGCCCT |
| | GACGGAGCTGGGCTACAAAATAGTCACAGGTGGTTCTGACAACCA |
| | TTTGATCCTTGTGGATCTCCGTTCCAAAGGCACAGATGGTGGAAG |
| | GGCTGAGAAGGTGCTAGAAGCCTGTTCTATTGCCTGCAACAAGAA |
| | CACCTGTCCAGGTGACAGAAGCGCTCTGCGGCCCAGTGGACTGCG |
| | GCTGGGGACCCCAGCACTGACGTCCCGTGGACTTTTGGAAAAAGA |
| | CTTCCAAAAAGTAGCCCACTTTATTCACAGAGGGATAGAGCTGAC |
| | CCTGCAGATCCAGAGCGACACTGGTGTCAGAGCCACCCTGAAAGA |
| | GTTCAAGGAGAGACTGGCAGGGGATAAGTACCAGGCGGCCGTGCA |
| | GGCTCTCCGGGAGGAGGTTGAGAGCTTCGCCTCTCTCTTCCCTCT |
| | GCCTGGCCTGCCTGACTTCTAAAGGAGCGGGCCCACTCTGGACCC |
| | ACCTGGCGCCACAGAGGAAGCTGCCTGCCGGAGGACCCCCACCTG |
| | AGAGATGGATGAGCTGCTCCAAAGGGGAACTGTTGACACTCGGGC |
| | CCTTTGAGGGGGTTTCTTTTGGACTTTTTTCATGTTTTCTTCACA |
| | AATCAAAATTTGTTTAAGTCTCATTGTTAGTAATTCTGGGACAGG |
| | TTATTAAAGGATTTAAATTTGAACCTGGCTTTCTCACAGCTGGAC |
| | ATAATTCTAGGAAAATAAAATACTATGTCGCCACTTGGTCATAAT |
| | CATTTAGATGGTGGTGTAGGGCAAAGCTGTTAGAAAGATTGTAGC |
| | GTTTTACTCTCCCTGGGCTTTCCTCCGCCTTGCTGCAACAGAGAG |
| | GAAATGCCCATGTCCACAGCTTGTACACACTGCCCCCTCACTATC |
| | TTGTTATCCAGTGGCATGCCAAAGGAGAACTGAATTAGCTTCTGA |
| | GGCTTCTGCTGTAAATCAGAAGTGTATGTTAGTCAAGAGTAAACA |
| | AGATGCACCCAGTATGGTGGGAGGGTTTTGCTGTCAGTAGCTCAA |
| | AGTATGGTGTAGAAATGGCCTCCTCCCTCCATCCTGGGAAGTCCC |
| | AGTCCCATCCTGGTGTGAGAATCAACCAGGCTTTCCTGCTCCACC |
| | TGAGATAACCAACTCCCTCCCGTAATCAGGAAGCCAAATGTCACC |
| | TTCCCAAAGAAATTTTATTTTCACGTAGCTGAAGTGCAAAACATA |
| | GATGACCATTTTTAATAAGCACAATCAAATTTTTAACCACAGAAT |
| | GTCTACAAGAATTATAGCTTTAAAAAATACAACCAATTTTTATAT |
| | TTCAAAAATATTTGAACTCAAATAAATTAATTTCTTAAAAAGTAA |
| | AAAAAAAAAAAAAAAAAAAA (SEQ ID NO: 26) |
| Human SHMT2 | MLYFSLFWAARPLQRCGQLVRMAIRAQHSNAAQTQTGEANRGWTG |
| Protein Sequence | QESLSDSDPEMWELLQREKDRQCRGLELIASENFCSRAALEALGS |
| serine | CLNNKYSEGYPGKRYYGGAEVVDEIELLCQRRALEAFDLDPAQWG |
| hydroxymethyltrans- | VNVQPYSGSPANLAVYTALLQPHDRIMGLDLPDGGHLTHGYMSDV |
| ferase, | KRISATSIFFESMPYKLNPKTGLIDYNQLALTARLFRPRLIIAGT |
| mitochondrial | SAYARLIDYARMREVCDEVKAHLLADMAHISGLVAAKVIPSPFKH |
| isoform 1 precursor | ADIVTTTTHKTLRGARSGLIFYRKGVKAVDPKTGREIPYTFEDRI |
| (NCBI Reference | NFAVFPSLQGGPHNHAIAAVAVALKQACTPMFREYSLQVLKNARA |
| Sequence: | MADALLERGYSLVSGGTDNHLVLVDLRPKGLDGARAERVLELVSI |
| NP_005403.2) | TANKNTCPGDRSAITPGGLRLGAPALTSRQFREDDFRRVVDFIDE |
| | GVNIGLEVKSKTAKLQDFKSFLLKDSETSQRLANLRQRVEQFARA |
| | FPMPGFDEH (SEQ ID NO: 27) |
| Human SHMT2 | ATAAAGAAAAAAGCGGTGAGTGGGCGAACTACAATTCCCAAAAGG |
| mRNA Sequence | CCACAAAGGGGCCACCACTACGCATGCGTAGATCCCTCCCGTTAG |
| serine | CTTTGGCGCCTCAGCGAGCTCTTCTCGCGCATGCGTTCTCCGAAC |
| hydroxymethyltrans- | GGTCTTCTTCCGACAGCTTGCTGCCCTAGACCAGAGTTGGTGGCT |
| ferase 2 | GGACCTCCTGCGACTTCCGAGTTGCGATGCTGTACTTCTCTTTGT |
| (mitochondrial) | TTTGGGCGGCTCGGCCTCTGCAGAGATGTGGGCAGCTGGTCAGGA |
| (SHMT2), nuclear | TGGCCATTCGGGCTCAGCACAGCAACGCAGCCCAGACTCAGACTG |
| gene encoding | GGGAAGCAAACAGGGGCTGGACAGGCCAGGAGAGCCTGTCGGACA |
| mitochondrial | GTGATCCTGAGATGTGGGAGTTGCTGCAGAGGGAGAAGGACAGGC |
| protein, transcript | AGTGTCGTGGCCTGGAGCTCATTGCCTCAGAGAACTTCTGCAGCC |
| variant 1 | GAGCTGCGCTGGAGGCCCTGGGGTCCTGTCTGAACAACAAGTACT |
| (NCBI Reference | CGGAGGGTTATCCTGGCAAGAGATACTATGGGGGAGCAGAGGTGG |
| Sequence: | TGGATGAAATTGAGCTGCTGTGCCAGCGCCGGGCCTTGGAAGCCT |
| NM_005412.5) | TTGACCTGGATCCTGCACAGTGGGGAGTCAATGTCCAGCCCTACT |
| | CCGGGTCCCCAGCCAACCTGGCCGTCTACACAGCCCTTCTGCAAC |
| | CTCACGACCGGATCATGGGGCTGGACCTGCCCGATGGGGGCCATC |
| | TCACCCACGGCTACATGTCTGACGTCAAGCGGATATCAGCCACGT |
| | CCATCTTCTTCGAGTCTATGCCCTATAAGCTCAACCCCAAAACTG |
| | GCCTCATTGACTACAACCAGCTGGCACTGACTGCTCGACTTTTCC |
| | GGCCACGGCTCATCATAGCTGGCACCAGCGCCTATGCTCGCCTCA |
| | TTGACTACGCCCGCATGAGAGAGGTGTGTGATGAAGTCAAAGCAC |
| | ACCTGCTGGCAGACATGGCCCACATCAGTGGCCTGGTGGCTGCCA |
| | AGGTGATTCCCTCGCCTTTCAAGCACGCGGACATCGTCACCACCA |
| | CTACTCACAAGACTCTTCGAGGGGCCAGGTCAGGGCTCATCTTCT |
| | ACCGGAAAGGGGTGAAGGCTGTGGACCCCAAGACTGGCCGGGAGA |
| | TCCCTTACACATTTGAGGACCGAATCAACTTTGCCGTGTTCCCAT |
| | CCCTGCAGGGGGGCCCCCACAATCATGCCATTGCTGCAGTAGCTG |
| | TGGCCCTAAAGCAGGCCTGCACCCCCATGTTCCGGGAGTACTCCC |
| | TGCAGGTTCTGAAGAATGCTCGGGCCATGGCAGATGCCCTGCTAG |
| | AGCGAGGCTACTCACTGGTATCAGGTGGTACTGACAACCACCTGG |
| | TGCTGGTGGACCTGCGGCCCAAGGGCCTGGATGGAGCTCGGGCTG |
| | AGCGGGTGCTAGAGCTTGTATCCATCACTGCCAACAAGAACACCT |
| | GTCCTGGAGACCGAAGTGCCATCACACCGGGCGGCCTGCGGCTTG |
| | GGGCCCCAGCCTTAACTTCTCGACAGTTCCGTGAGGATGACTTCC |
| | GGAGAGTTGTGGACTTTATAGATGAAGGGGTCAACATTGGCTTAG |
| | AGGTGAAGAGCAAGACTGCCAAGCTCCAGGATTTCAAATCCTTCC |
| | TGCTTAAGGACTCAGAAACAAGTCAGCGTCTGGCCAACCTCAGGC |
| | AACGGGTGGAGCAGTTTGCCAGGGCCTTCCCCATGCCTGGTTTTG |
| | ATGAGCATTGAAGGCACCTGGGAAATGAGGCCCACAGACTCAAAG |
| | TTACTCTCCTTCCCCCTACCTGGGCCAGTGAAATAGAAAGCCTTT |
| | CTATTTTTTGGTGCGGGAGGGAAGACCTCTCACTTAGGGCAAGAG |
| | CCAGGTATAGTCTCCCTTCCCAGAATTTGTAACTGAGAAGATCTT |
| | TTCTTTTTCCTTTTTTTGGTAACAAGACTTAGAAGGAGGGCCCAG |
| | GCACTTTCTGTTTGAACCCCTGTCATGATCACAGTGTCAGAGACG |
| | CGTCCTCTTTCTTGGGGAAGTTGAGGAGTGCCCTTCAGAGCCAGT |
| | AGCAGGCAGGGGTGGGTAGGCACCCTCCTTCCTGTTTTTATCTAA |
| | TAAAATGCTAACCTGCCCTGAGTTTCCATTACTGTGGGTGGGGTT |
| | CCCCTGGGCCAAACAGTGATTTGTCTCCCTCAATGTGTACACCGC |
| | TCCGCTCCCACCACCGCTACCACAAGGACCCCCGGGGCTGCAGCC |
| | TCCTCTTTCTGTCTCTGATCAGAGCCGACACCAGACGTGATTAGC |
| | AGGCGCAGCAAATTCAATTTGTTAAATGAAATTGTATTTTGCCCA |
| | (SEQ ID NO: 28) |
| Human SLC25A32 | MTGQGQSASGSSAWSTVFRHVRYENLIAGVSGGVLSNLALHPLDL |
| Protein Sequence | VKIRFAVSDGLELRPKYNGILHCLTTIWKLDGLRGLYQGVTPNIW |
| mitochondrial folate | GAGLSWGLYFFFYNAIKSYKTEGRAERLEATEYLVSAAEAGAMTL |
| transporter/carrier | CITNPLWVTKTRLMLQYDAVVNSPHRQYKGMFDTLVKIYKYEGVR |
| (NCBI Reference | GLYKGFVPGLFGTSHGALQFMAYELLKLKYNQHINRLPEAQLSTV |
| Sequence: | EYISVAALSKIFAVAATYPYQVVRARLQDQHMFYSGVIDVITKTW |
| NP_110407.2) | RKEGVGGFYKGIAPNLIRVTPACCITFVVYENVSHFLLDLREKRK |
| | (SEQ ID NO: 29) |
| Human SLC25432 | ACTCGCGGAGCGGCGGCCTGCTGGCTCAACTGGATCCTGCGCCGC |
| mRNA Sequence | TCCGTAGTTTTGCCGGCAAACGTTAGCAAGGGGCGGTTCTTTAGC |
| solute carrier | TGTGCAGTCGCTTCCGCGTCCGTGGGCTGGAGCATTTGTGGGCGA |
| family 25 | GGCAGGGCGGAGACTCGGGAGAGGCTGGGACCTCCCCTCCATCGC |
| (mitochondrial | GCTTTCCGCCGGCGTGACGTAGTGTCTGTGCCCCGTTCTTGCCCC |
| folate carrier), | CTCAGTACTAGAGTCTCCGGCTTCGCTCACGCGCCTTGGGCATAA |
| member 32 | GAGTCCTCTCGTTGGTCCCGGAGGTGGGGTTGCGCTCACAAGGGG |
| (SLC25A32), nuclear | CGACCGTCGCCACGGTGGCGGCCACTGCATCGCGTCCCACCTCCG |
| gene encoding | CGGCCCTGGGCGCCGTGGTGTCGACGGGCCCCGAGCCTATGACGG |
| mitochondrial | GCCAGGGCCAGTCGGCGTCCGGGTCGTCGGCGTGGAGCACGGTAT |
| protein, transcript | TCCGCCACGTCCGGTATGAGAACCTGATAGCGGGCGTGAGCGGCG |
| variant 1 | GCGTCTTATCCAACCTTGCGCTGCATCCGCTCGACCTCGTGAAGA |
| (NCBI Reference | TCCGCTTCGCCGTGAGTGATGGATTGGAACTGAGACCGAAATATA |
| Sequence: | ATGGAATTTTACATTGCTTGACTACCATTTGGAAACTTGATGGAC |
| NM_030780.4) | TACGGGGACTTTATCAAGGAGTAACCCCAAATATATGGGGTGCAG |
| | GTTTATCCTGGGGACTCTACTTTTTCTTTTACAATGCCATCAAGT |
| | CATATAAAACAGAAGGAAGAGCTGAACGTTTAGAGGCAACAGAAT |
| | ACCTTGTCTCAGCTGCTGAAGCTGGAGCCATGACCCTCTGCATTA |
| | CAAACCCATTATGGGTAACAAAAACTCGCCTTATGTTACAGTATG |
| | ATGCTGTTGTTAACTCCCCACACCGACAATATAAAGGAATGTTTG |
| | ATACACTTGTGAAAATATATAAGTATGAAGGTGTGCGTGGATTAT |
| | ATAAGGGATTTGTTCCTGGGCTGTTTGGAACATCGCATGGTGCCC |
| | TTCAGTTTATGGCATATGAATTGCTGAAGTTGAAGTACAACCAGC |
| | ATATCAATAGATTACCAGAAGCCCAGTTGAGCACAGTAGAATATA |
| | TATCTGTTGCAGCACTATCCAAAATATTTGCTGTCGCAGCAACAT |
| | ACCCATATCAAGTCGTAAGAGCTCGTCTTCAGGATCAACACATGT |
| | TTTACAGTGGTGTAATAGATGTAATCACAAAGACATGGAGGAAAG |
| | AAGGCGTCGGTGGATTTTACAAGGGAATTGCTCCTAATTTGATTA |
| | GAGTGACTCCAGCCTGCTGTATTACCTTTGTGGTATATGAAAACG |
| | TCTCACATTTTTTACTTGACCTTAGAGAAAAGAGAAAGTAAGCTC |
| | AAAGAGGACAATTCCAGTATATCTGCCCAAGGCAGCAACAAGCTC |
| | TTTTGTGTTTAAGGCATAAAAGAAGAATTCTGCATAGAAACATGG |
| | CTCATATTCGAAATTGCTCTATAGTCATTAGAAGCCAGAGAACTG |
| | CTAAGTCTCCTGCAATGTTTTTCTTGCTTTTTGCCTTCCCCATAT |
| | ATATGGAACTTGGCTACCTCTGCCTGAAATGGCTGCCATCAACAC |
| | AATGTTAAAACTGACACGAAGGATAGAGTTTCACAGATTTCTACG |
| | TTTTATTGGTGGAAGCTGATTTGCAACATTTGCTAAATGGATTAG |
| | ATGAATGTACTTCTTTTTGTGAGCTTACTTGCCTGGATTGCTTTA |
| | AAATTAACCTTTGTGCAATACCAAGAAAATAGCTCTTTAAAAGAA |
| | TGTCTTTGTATGTCTCAAGGTAAATTAAGGATTTACTGAATAAGG |
| | TGTTGACCAAATCCAGACCATTTTATTTTATTTTTTTATTTATTT |
| | ATTTTTTGAGATGGAGTCTTGCTTTGTCGCCCAGGCTGGAGTGCA |
| | GTGGCGTGATCTCAGCTCACTGCAACCTCCACCTCCCGGGTTCAC |
| | GCCATTCTCCTGCCTCAGCCTCCTGAGTAGCTGGGACTACAGGCA |
| | CCTGCCACCACGCCTGGCTAACTTTTTTTTATATTTTGAGTAGAA |
| | ATGGGGTTTCACCATGTTAGCCAGGATGGTCTCAATCTCCTGACC |
| | TTGTGATCCGCCTGCCTTGGCCTCCCAAAGTGCTGGGATTACAGG |
| | CGTGAGCCACTGCGCCTGGCCAGACCATTTTAGAATTGGGAAATT |
| | TTAGTGAGAAAAAATGCACTGTAAATATGCTTTAGTTTTAATTCA |
| | GTTGGGATGCACTACCTAGCGAAAATTGAGAAACTATATACTTCT |
| | CAGAGAAATATCTGACATCTATTGTCATTCCATTGCTATTTTTTT |
| | TCCCCAGAGACTTCCATAATTTAAAATAAAATCCTAGATCCAGTT |
| | CTTGTTTTTTGGCATAAATACTTAATCTATTTTAAATTTATAAAA |
| | TCTGAGCTTCTAGGATCCAGCTGTGTCAACCTTTATTTAGCATAT |
| | ATAACTATAAATCACTTATTACAGATGCTAAATAGATCACCTTTT |
| | ACAGATGCTGAAATGTTTGGGATATGTTTGTTGACAAGGTAAATG |
| | GAAATGAGAAACTTTATACTTCAGTTTTCAGATATATGGATCTAG |
| | ATCCCAAATAAATGATTAATCTTCATTGGTTTCTCAAATTCAGGT |
| | TGAAATACAAATTAATAGCCTTTATTGATTTTACTTTTATGAGTC |
| | ATTGTAGACATCTATAAATATAAAAGGGCCTGTACCCAAAGGATG |
| | CCAGAATACTAGTATTTTTATTTATCGTAAACATCCACGAGTGCT |
| | GTTGCACTACCATCTATTTGTTGTAAATAAAAGTGTTGTTTTCAA |
| | AGCCATCTTTAAATAGTTCTTTAAAAATAGGTCTTTTTTTTATAT |
| | TTTGGAAAAGGCATTGTTTTTAAAGTAAAGATAAAATGGTAAGTA |
| | CCTAATTGTATTTACTGTAATATCTTATAACATGCAGATGAATGC |
| | TTTATAAGTTAAATATGATGTATTTTTTCATACTTCTGGATTATA |
| | CTATAATTCATATGAAATCTTGATATTAGTCCCCACACGGAAAAA |
| | GTGAACTGCAGTTGATATTTGGTGTTTAAGATAGCACCATTGTTT |
| | AAATACCGCCTATGTACTCCCAAATGAATAAAACATAATTCTTGT |
| | CCTCTGAGAGCATAAAAAAAAAAAAAAA (SEQ ID NO: |
| | 30) |

Reduced Folate Metabolism and Neurological Dysfunction or Disorders

The present invention encompasses the recognition that folate pathway loss-of-function mutations (e.g., reduced folate metabolism) are associated with a risk or susceptibility to a neurological dysfunction or disorder. In some embodiments, a neurological dysfunction or disorder is any dysfunction or disorder that result in impairment of neuronal mediated functions and includes disorders of the central nervous system (e.g., the brain, spinal cord) as well as the peripheral nervous system. In some embodiments, a neurological dysfunction or disorder comprises autism. In some embodiments, a neurological dysfunction or disorder comprises Pediatric Acute-onset Neuropsychiatric Syndrome (PANS). In some embodiments, a neurological dysfunction or disorder comprises abnormal autonomic activity. In some embodiments, a neurological dysfunction or disorder comprises functional gastrointestinal disorders (e.g., GI dysmotility, gastroesophageal reflux disease (i.e., GERD), small bowel disease, large bowel disease, irritable bowel syndrome, constipation, cyclic vomiting syndrome, etc.). In some embodiments, a neurological dysfunction or disorder comprises chronic pain disorders (e.g., migraine, abdominal pain, myalgia, etc.). In some embodiments, a neurological dysfunction or disorder comprises chronic fatigue disorders. In some embodiments, a neurological dysfunction or disorder comprises autistic spectrum disorders. In some embodiments, a neurological dysfunction or disorder comprises psychiatric disorders. In some embodiments, a neurological dysfunction or disorder comprises cognitive dysfunction and/or decline. In some embodiments, a neurological dysfunction or disorder comprises episodic encephalopathy. In some embodiments, a neurological dysfunction or disorder comprises episodic dementia/psychosis.

In some embodiments, a risk of a neurological dysfunction or disorder comprises a risk from 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference. In some embodiments, a reference comprises an average occurrence of a neurological dysfunction or disorder in a population. In some embodiments, a reference comprises a statistical occurrence of a neurological dysfunction or disorder deemed to be acceptable or unavoidable in a population by medical professionals.

Reduced Folate Metabolism and Mitochondrial Dysfunction or Disorders

The present invention encompasses the recognition that folate pathway loss-of-function mutations (e.g., reduced folate metabolism) are associated with a risk or susceptibility to a mitochondrial dysfunction or disorder. As used herein, the term “mitochondrial diseases or disorders” refers to a complex variety of symptoms. In some embodiments, a mitochondrial dysfunction or disorder is any dysfunction or disorder that affects the mitochondria, the organelles that generate energy for the cell. In some embodiments, a mitochondrial dysfunction or disorder includes, but is not limited to muscle weakness, muscle cramps, seizures, food reflux, learning disabilities, deafness, short stature, paralysis of eye muscles, diabetes, cardiac problems and stroke-like episodes. The symptoms can range in severity from life-threatening to almost unnoticeable, sometimes taking both extremes in members of the same family. Because some people have specific subsets of these symptoms, clinical researchers have grouped those that occur together into “syndromes,” producing a bewildering array of descriptive acronyms such as MELAS (mitochondrial encephalomyopathy with lactic acidosis and stroke-like episodes) or MERFF (myoclonus epilepsy with ragged red fibers). This term also includes disorders such as Kearns-Sayre syndrome (KSS), Leigh's syndrome, maternally inherited Leigh's syndrome (MILS), Myogastrointestinal encephalomyopathy (MNGIE), Neuropathy, ataxia and retinitis pigmentosa (NARP), Friedreich's ataxia (FRDA), amyotrophic lateral sclerosis (ALS) and other motor neuron diseases, Huntington's disease, macular degeneration, epilepsy, Alzheimer's, Leber's hereditary optic neuropathy (LHON), Progressive external ophthalmoplegia (PEO), and Pearson syndrome.

In some embodiments, a mitochondrial dysfunction or disorder may affect the central or peripheral nervous system. In some embodiments, a mitochondrial dysfunction or disorder comprises abnormal autonomic activity. In some embodiments, a mitochondrial dysfunction or disorder comprises functional gastrointestinal disorders (e.g., GI dysmotility, gastroesophageal reflux disease (i.e., GERD), small bowel disease, large bowel disease, irritable bowel syndrome, constipation, cyclic vomiting syndrome, etc.). In some embodiments, a mitochondrial dysfunction or disorder comprises chronic pain disorders (e.g., migraines, abdominal pain, myalgia, etc.). In some embodiments, a mitochondrial dysfunction or disorder comprises chronic fatigue disorders. In some embodiments, a mitochondrial dysfunction or disorder comprises chronic fatigue disorders. In some embodiments, a mitochondrial dysfunction or disorder comprises autistic spectrum disorders. In some embodiments, a mitochondrial dysfunction or disorder comprises psychiatric disorders. In some embodiments, a mitochondrial dysfunction or disorder comprises cognitive dysfunction and/or decline. In some embodiments, a mitochondrial dysfunction or disorder comprises episodic encephalopathy. In some embodiments, a mitochondrial dysfunction or disorder comprises episodic dementia/psychosis.

In some embodiments, a risk of a mitochondrial dysfunction or disorder comprises a risk from 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference. In some embodiments, a reference comprises an average occurrence of a mitochondrial dysfunction or disorder in a population. In some embodiments, a reference comprises a statistical occurrence of a mitochondrial dysfunction or disorder deemed to be acceptable or unavoidable in a population by medical professionals.

Folate Pathway Loss-of-Function Mutations

The present invention encompasses the recognition that a loss-of-function mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 can be associated with an altered risk of or suffering from a mitochondrial dysfunction or disorder, autism, and/or Pediatric Acute-onset Neuropsychiatric Syndrome (PANS).

In some embodiments, a loss-of-function mutation is in the regulatory sequence of the ALDH1L1 gene. In some embodiments, the loss-of-function mutation is in the coding sequence of the ALDH1L1 gene. In some embodiments, the loss-of-function mutation comprises a mutation of amino acid residues 23, 64-107, 117, 333, 448, 524, 666, 760, 771 and/or 876 of ALDH1L1 (SEQ ID NO: 1). In some embodiments, the loss-of-function mutation is or comprises a mutation selected from the group consisting of 23G>D, 117S>L, 333R>Q, 4485>N, 524G>S, 666N>K, 760E>K771T>A, 876K>R, frame shift p.Ala107Profs64X, and combinations thereof.

In some embodiments, the loss-of-function mutation in nuclear DNA that encodes ALDH1L1 causes reduced expression of a ALDH1L1 gene product. In some embodiments, reduced expression of a ALDH1L1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type ALDH1L1 gene.

In some embodiments, a loss-of-function mutation is in the regulatory sequence of the ALDH1L2 gene. In some embodiments, the loss-of-function mutation is in the coding sequence of the ALDH1L2 gene. In some embodiments, the loss-of-function mutation comprises a mutation of amino acid residues 204, 603, 748, 796, 833 and/or 918 of ALDH1L2 (SEQ ID NO: 3). In some embodiments, the loss-of-function mutation is or comprises a mutation selected from the group consisting of 204L>F, 603W>X, 748V>A, 796G>R, 833T>I, 918T>M, and combinations thereof.

In some embodiments, the loss-of-function mutation in nuclear DNA that encodes ALDH1L2 causes reduced expression of a ALDH1L2 gene product. In some embodiments, reduced expression of a ALDH1L2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type ALDH1L2 gene.

In some embodiments, a loss-of-function mutation is in the regulatory sequence of the FOLR1 gene. In some embodiments, the loss-of-function mutation is in the coding sequence of the FOLR1 gene. In some embodiments, the loss-of-function mutation comprises a mutation of amino acid residue 98 of FOLR1 (SEQ ID NO: 5). In some embodiments, the loss-of-function mutation is or comprises a mutation consisting of 98R>W.

In some embodiments, the loss-of-function mutation in nuclear DNA that encodes FOLR1 causes reduced expression of a FOLR1 gene product. In some embodiments, reduced expression of a FOLR1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type FOLR1 gene.

In some embodiments, a loss-of-function mutation is in the regulatory sequence of the FPGS gene. In some embodiments, the loss-of-function mutation is in the coding sequence of the FPGS gene. In some embodiments, the loss-of-function mutation comprises a mutation of amino acid residues 50, 85, 162 and/or 466 of FPGS (SEQ ID NO: 7). In some embodiments, the loss-of-function mutation is or comprises a mutation selected from the group consisting of 50R>C, 85R>W, 162R>Q, 466R>C, and combinations thereof

In some embodiments, the loss-of-function mutation in nuclear DNA that encodes FPGS causes reduced expression of a FPGS gene product. In some embodiments, reduced expression of a FPGS gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type FPGS gene.

In some embodiments, a loss-of-function mutation is in the regulatory sequence of the GCSH gene. In some embodiments, the loss-of-function mutation is in the coding sequence of the GCSH gene. In some embodiments, the loss-of-function mutation comprises a mutation of amino acid residue 84 of GCSH (SEQ ID NO: 9). In some embodiments, the loss-of-function mutation is or comprises a mutation consisting of 84Y>H.

In some embodiments, the loss-of-function mutation in nuclear DNA that encodes GCSH causes reduced expression of a GCSH gene product. In some embodiments, reduced expression of a GCSH gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type GCSH gene.

In some embodiments, a loss-of-function mutation is in the regulatory sequence of the GLDC gene. In some embodiments, the loss-of-function mutation is in the coding sequence of the GLDC gene. In some embodiments, the loss-of-function mutation comprises a mutation of amino acid residues 18, 147, 503, 675, 705, 716, 895, 937 and/or 966 of GLDC (SEQ ID NO: 11). In some embodiments, the loss-of-function mutation is or comprises a mutation selected from the group consisting of 18G>C, 147I>M, 503E>A, 675N>K, 705V>M, 716L>H, 895M>V, 937R>L, 966Q>H, and combinations thereof.

In some embodiments, the loss-of-function mutation in nuclear DNA that encodes GLDC causes reduced expression of a GLDC gene product. In some embodiments, reduced expression of a GLDC gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type GLDC gene.

In some embodiments, a loss-of-function mutation is in the regulatory sequence of the MTHFD1 gene. In some embodiments, the loss-of-function mutation is in the coding sequence of the MTHFD1 gene. In some embodiments, the loss-of-function mutation comprises a mutation of amino acid residue 830 of MTHFD1 (SEQ ID NO: 13). In some embodiments, the loss-of-function mutation is or comprises a mutation consisting of 830A>V.

In some embodiments, the loss-of-function mutation in nuclear DNA that encodes MTHFD I causes reduced expression of a MTHFD1 gene product. In some embodiments, reduced expression of a MTHFD 1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD1 gene.

In some embodiments, a loss-of-function mutation is in the regulatory sequence of the MTHFD1L gene. In some embodiments, the loss-of-function mutation is in the coding sequence of the MTHFD1L gene. In some embodiments, the loss-of-function mutation comprises a mutation of amino acid residues 31, 520, 564 and/or 949 of MTHFD1L (SEQ ID NO: 15). In some embodiments, the loss-of-function mutation is or comprises a mutation selected from the group consisting of 31A>G, 520Y>C, 564R>H, 949G>R, and combinations thereof.

In some embodiments, the loss-of-function mutation in nuclear DNA that encodes MTHFD1L causes reduced expression of a MTHFD1L gene product. In some embodiments, reduced expression of a MTHFD1L gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD1L gene.

In some embodiments, a loss-of-function mutation is in the regulatory sequence of the MTHFD2 gene. In some embodiments, the loss-of-function mutation is in the coding sequence of the MTHFD2 gene. In some embodiments, the loss-of-function mutation comprises a mutation of amino acid residue 263 of MTHFD2 (SEQ ID NO: 17). In some embodiments, the loss-of-function mutation is or comprises a mutation consisting of 263D>G.

In some embodiments, the loss-of-function mutation in nuclear DNA that encodes MTHFD2 causes reduced expression of a MTHFD2 gene product. In some embodiments, reduced expression of a MTHFD2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD2 gene.

In some embodiments, a loss-of-function mutation is in the regulatory sequence of the MTHFD2L gene. In some embodiments, the loss-of-function mutation is in the coding sequence of the MTHFD2L gene. In some embodiments, the loss-of-function mutation comprises a mutation of amino acid residues 161 and/or 210 of MTHFD2L (SEQ ID NO: 19). In some embodiments, the loss-of-function mutation is or comprises a mutation selected from the group consisting of 161G>E, 210V>L, and combinations thereof.

In some embodiments, the loss-of-function mutation in nuclear DNA that encodes MTHFD21, causes reduced expression of a MTHFD2L gene product. In some embodiments, reduced expression of a MTHFD2L gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD2L gene.

In some embodiments, a loss-of-function mutation is in the regulatory sequence of the MTHFS gene. In some embodiments, the loss-of-function mutation is in the coding sequence of the MTHFS gene. In some embodiments, the loss-of-function mutation comprises a mutation of amino acid residues 133 and/or 174 of MTHFS (SEQ ID NO: 21). In some embodiments, the loss-of-function mutation is or comprises a mutation selected from the group consisting of 133L>Q, 174E>K, and combinations thereof.

In some embodiments, the loss-of-function mutation in nuclear DNA that encodes MTHFS causes reduced expression of a MTHFS gene product. In some embodiments, reduced expression of a MTHFS gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFS gene.

In some embodiments, a loss-of-function mutation is in the regulatory sequence of the MTRR gene. In some embodiments, the loss-of-function mutation is in the coding sequence of the MTRR gene. In some embodiments, the loss-of-function mutation comprises a mutation of amino acid residues 317 and/or 517 of MTRR (SEQ ID NO: 23). In some embodiments, the loss-of-function mutation is or comprises a mutation selected from the group consisting of 317I>T, 517T>A, and combinations thereof.

In some embodiments, the loss-of-function mutation in nuclear DNA that encodes MTRR causes reduced expression of a MTRR gene product. In some embodiments, reduced expression of a MTRR gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTRR gene.

In some embodiments, a loss-of-function mutation is in the regulatory sequence of the SHMT1 gene. In some embodiments, the loss-of-function mutation is in the coding sequence of the SHMT1 gene. In some embodiments, the loss-of-function mutation comprises a mutation of amino acid residues 1, 191 and/or 344 of SHMT 1 (SEQ ID NO: 25). In some embodiments, the loss-of-function mutation is or comprises a mutation selected from the group consisting of 1M>R, 1M>K, 191R>C, 344E>Q, and combinations thereof.

In some embodiments, the loss-of-function mutation in nuclear DNA that encodes SHMT1 causes reduced expression of a SHMT1 gene product. In some embodiments, reduced expression of a SHMT1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SHMT1 gene.

In some embodiments, a loss-of-function mutation is in the regulatory sequence of the SHMT1 gene. In some embodiments, the loss-of-function mutation is in the coding sequence of the SHMT2 gene. In some embodiments, the loss-of-function mutation comprises a mutation of amino acid residues 193 and/or 327 of SHMT2 (SEQ ID NO: 27). In some embodiments, the loss-of-function mutation is or comprises a mutation selected from the group consisting of 193R>Q, 327R>Q, and combinations thereof.

In some embodiments, the loss-of-function mutation in nuclear DNA that encodes SHMT2 causes reduced expression of a SHMT2 gene product. In some embodiments, reduced expression of a SHMT2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SHMT2 gene.

In some embodiments, a loss-of-function mutation is in the regulatory sequence of the SLC25A32 gene. In some embodiments, the loss-of-function mutation is in the coding sequence of the SLC25A32 gene. In some embodiments, the loss-of-function mutation comprises a mutation of amino acid residues 163 and/or 300 of SLC25A32 (SEQ ID NO: 29). In some embodiments, the loss-of-function mutation is or comprises a mutation selected from the group consisting of 163Y>C, 300Y>C, and combinations thereof.

In some embodiments, the loss-of-function mutation in nuclear DNA that encodes SLC25A32 causes reduced expression of a SLC25A32 gene product. In some embodiments, reduced expression of a SLC25A32 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SLC25A32 gene.

Methods of quantifying levels of RNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (1998. Current Protocols in Molecular Biology. Wiley: New York).

In some embodiments, the loss-of-function mutation causes reduced activity of a ALDH1L1 gene product. In some embodiments, reduced activity of a ALDH1L1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type ALDH1L1 gene.

In some embodiments, the loss-of-function mutation causes reduced activity of a ALDH 1 L2 gene product. In some embodiments, reduced activity of a ALDH1 L2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type ALDH1L2 gene.

In some embodiments, the loss-of-function mutation causes reduced activity of a FOLR1 gene product. In some embodiments, reduced activity of a FOLR1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type FOLR1 gene.

In some embodiments, the loss-of-function mutation causes reduced activity of a FPGS gene product. In some embodiments, reduced activity of a FPGS gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type FPGS gene.

In some embodiments, the loss-of-function mutation causes reduced activity of a GCSH gene product. In some embodiments, reduced activity of a GCSH gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type GCSH gene.

In some embodiments, the loss-of-function mutation causes reduced activity of a GLDC gene product. In some embodiments, reduced activity of a GLDC gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type GLDC gene.

In some embodiments, the loss-of-function mutation causes reduced activity of a MTHFD I gene product. In some embodiments, reduced activity of a MTHFD1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD1 gene.

In some embodiments, the loss-of-function mutation causes reduced activity of a MTHFD1L gene product. In some embodiments, reduced activity of a MTHFD1L gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD1L gene.

In some embodiments, the loss-of-function mutation causes reduced activity of a MTHFD2 gene product. In some embodiments, reduced activity of a MTHFD2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD2 gene.

In some embodiments, the loss-of-function mutation causes reduced activity of a MTHFD2L gene product. In some embodiments, reduced activity of a MTHFD2L gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFD2L gene.

In some embodiments, the loss-of-function mutation causes reduced activity of a MTHFS gene product. In some embodiments, reduced activity of a MTHFS gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTHFS gene.

In some embodiments, the loss-of-function mutation causes reduced activity of a MTRR gene product. In some embodiments, reduced activity of a MTRR gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type MTRR gene.

In some embodiments, the loss-of-function mutation causes reduced activity of a SHMT1 gene product. In some embodiments, reduced activity of a SHMT1 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SHMT1 gene.

In some embodiments, the loss-of-function mutation causes reduced activity of a SHMT2 gene product. In some embodiments, reduced activity of a SHMT2 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SHMT2 gene.

In some embodiments, the loss-of-function mutation causes reduced activity of a SLC25A32 gene product. In some embodiments, reduced activity of a SLC25A32 gene product comprises a reduction of 1, 2, 3, 4, 5, 6, 7, 8, 9, 10, 15, 20, 25, 30, 35, 40, 45, 50, 55, 60, 65, 70, 75, 80, 85, 90, 95, 100% or more relative to a reference. In some embodiments, a reference is a sample from an individual without a mitochondrial dysfunction or disorder, autism, and/or Pediatric Acute-onset Neuropsychiatric Syndrome (PANS). In some embodiments, a reference is a sample from an individual known to have a wild type SLC25A32 gene.

Diagnosis of Neurological and Mitochondrial Dysfunctions or Disorders

In some embodiments, the present invention provides methods of classifying an individual at risk of or suffering from a mitochondrial dysfunction or disorder, autism, and/or Pediatric Acute-onset Neuropsychiatric Syndrome (PANS). In general, such methods comprise obtaining a sample of nuclear DNA from the individual; processing the sample to determine whether the individual possesses a mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32; and classifying the individual as one that does or does not possess a mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32.

In some embodiments, an individual at risk of or suffering from a mitochondrial dysfunction or disorder, autism, and/or Pediatric Acute-onset Neuropsychiatric Syndrome (PANS) is a non-human animal. In some embodiments, a non-human animal is a mouse. In some embodiments, a non-human animal is a rat. In some embodiments, a non-human animal is a dog. In some embodiments, a non-human animal is a non-human primate. In some embodiments, an individual is a human. In some embodiments, a sample is obtained from an individual harboring an ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, or SLC25A32 mutation, and/or combinations therein. In some embodiments, a sample is obtained from an individual harboring a loss-of-function mutation in nuclear DNA that encodes ALDH1L1, ALDH1L2, FOLR1FPGS, GCSH, GLDC, MTHFD1, MTHFD1L, MTHFD2, MTHFD2L, MTHFS, MTRR, SHMT1, SHMT2, and/or SLC25A32 described herein.

In some embodiments, an individual at risk of or suffering from a neurological dysfunction or disorder suffers from a mitochondrial dysfunction or disorder. Many neurological dysfunctions and disorders are mitochondria driven and share common genomic malfunctions with mitochondrial dysfunctions and disorders. Mitochondrial dysfunction or disorders are degenerative diseases due to various mechanisms such as abnormality of mitochondrial DNA (deletion, point mutation, and duplication), abnormality of cellular DNA encoding mitochondrial enzymes or complex polymeric mitochondrial components, or can be induced by toxic substances or pharmaceutical products. When mitochondria-associated genes are damaged because of these reasons, various biochemical abnormalities occur.

In some embodiments, an individual possessing a mutation in their nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1 SHMT2, and/or SLC25A32 does not possesses heteroplasmic mitochondrial DNA variants. In some embodiments, an individual possessing a mutation in their nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 also possesses one or more homoplasmic mitochondrial DNA variants. Methods for sequencing mitochondrial DNA are well known in the art.

In some embodiments, a sample is any sample comprising ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 nuclear DNA. In some embodiments, a sample comprises cells from which nuclear DNA (e.g., not mitochondrial DNA) is or can be obtained. In some embodiments, a sample comprises cells from which mitochondrial DNA is or can be obtained. In some embodiments, a sample comprises isolated nucleic acids. In some embodiments, a sample comprises genomic DNA. In some embodiments, a sample comprises human genomic DNA.

In some embodiments, processing comprises processing a sample to detect a sequence of nuclear DNA that encodes ALDH1L1, ALDH1L2, FOLR1, FPGS, GOSH, GLDC, MTHFD1, MTHFD1L, MTHFD2, MTHFD2L, MTHFS, MTRR, SHMT1, SHMT2, and/or SLC25A32. In some embodiments, processing a sample comprises amplifying a target nucleic acid region of human genomic DNA encompassing a region that encodes the ALDH1L1 polypeptide, ALDH1L2 polypeptide, FOLR1 polypeptide, FPGS polypeptide, GCSH polypeptide, GLDC polypeptide, MTHFD1 polypeptide, MTHFD1L, polypeptide, MTHFD2 polypeptide, MTHFD2L, polypeptide, MTHFS polypeptide, MTRR polypeptide, SHMT1 polypeptide, SHMT2 polypeptide, and/or SLC25A32 polypeptide wherein said region includes one or more sites of loss-of-function mutations that are associated with a mitochondrial dysfunction or disorder, autism, and/or Pediatric Acute-onset Neuropsychiatric Syndrome (PANS). In some embodiments, amplifying comprises contacting the human genomic DNA with a 5′ primer under conditions such that hybridization and extension of the target nucleic acid region occur in a forward direction. In some embodiments, amplifying further comprises contacting the human genomic DNA with a 3′ primer under conditions such that hybridization and extension of the target nucleic acid region occur in a reverse direction.

Nucleic acid amplification methods are well known in the art and include, but are not limited to, the Polymerase Chain Reaction (or PCR, described, for example, in U.S. Pat. Nos. 4,683,195, 4,683,202 and 4,889,818, each of which is incorporated herein by reference in its entirety). In its simplest form, PCR is an in vitro method for the enzymatic synthesis of specific DNA sequences, using two primers that hybridize to opposite strands and flank the region of interest in the target DNA. A plurality of reaction cycles, each cycle comprising: a denaturation step, an annealing step, and a polymerization step, results in the exponential accumulation of a specific DNA fragment. The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq) which are available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs.

In some embodiments, the one or more sites of loss-of-function mutations correspond to amino acids 23, 64-107, 117, 333, 448, 524, 666, 760, 771 and/or 876 of an ALDH1L1 gene product. In some embodiments, the loss-of-function mutations are selected from the group consisting of 23G>D, 1175>L, 333R>Q, 4485>N, 524G>S, 666N>K, 760E>K771T>A, 876K>R, frame shift p.Ala107Profs64X, and combinations thereof.

In some embodiments, the one or more sites of loss-of-function mutations correspond to amino acids 204, 603, 748, 796, 833 and/or 918 of an ALDH1L2 gene product. In some embodiments, the loss-of-function mutations are selected from the group consisting of 204L>F, 603W>X, 748V>A, 796G>R, 833T>I, 918T>M, and combinations thereof.

In some embodiments, the one or more sites of loss-of-function mutations correspond to amino acid 98 of a FOLR1 gene product. In some embodiments, the loss-of-function mutations comprise a mutation consisting of 98R>W.

In some embodiments, the one or more sites of loss-of-function mutations correspond to amino acids 50, 85, 162 and/or 466 of a FPGS gene product. In some embodiments, the loss-of-function mutations are selected from the group consisting of 50R>C, 85R>W, 162R>Q, 466R>C, and combinations thereof.

In some embodiments, the one or more sites of loss-of-function mutations correspond to amino acid 84 of an GCSH gene product. In some embodiments, the loss-of-function mutations comprise a mutation consisting of 84Y>H.

In some embodiments, the one or more sites of loss-of-function mutations correspond to amino acids 18, 147, 503, 675, 705, 716, 895, 937 and/or 966 of a GLDC gene product. In some embodiments, the loss-of-function mutations are selected from the group consisting of 18G>C, 147I>M, 503E>A, 675N>K, 705V>M, 716L>H, 895M>V, 937R>L, 966Q>H, and combinations thereof.

In some embodiments, the one or more sites of loss-of-function mutations correspond to amino acid 830 of a MTHFD I gene product. In some embodiments, the loss-of-function mutations comprise a mutation consisting of 830A>V.

In some embodiments, the one or more sites of loss-of-function mutations correspond to amino acids 31, 520, 564 and/or 949 of a MTHFD1L gene product. In some embodiments, the loss-of-function mutations are selected from the group consisting of 31A>G, 520Y>C, 564R>H, 949G>R, and combinations thereof.

In some embodiments, the one or more sites of loss-of-function mutations correspond to amino acid 263 of a MTHFD2 gene product. In some embodiments, the loss-of-function mutations comprises a mutation consisting of 263D>G.

In some embodiments, the one or more sites of loss-of-function mutations correspond to amino acids 161 and/or 210 of a MTHFD2 L gene product. In some embodiments, the loss-of-function mutations are selected from the group consisting of 161G>E, 210V>L, and combinations thereof.

In some embodiments, the one or more sites of loss-of-function mutations correspond to amino acids 133 and/and 174 of a MTHFS gene product. In some embodiments, the loss-of-function mutations are selected from the group consisting of 133L>Q, 174E>K, and combinations thereof.

In some embodiments, the one or more sites of loss-of-function mutations correspond to amino acids 317 and/or 517 of a MTRR gene product. In some embodiments, the loss-of-function mutations are selected from the group consisting of 317I>T, 517T>A, and combinations thereof.

In some embodiments, the one or more sites of loss-of-function mutations correspond to amino acids 1, 191 and/or 344 of a SHMT1 gene product. In some embodiments, the loss-of-function mutations are selected from the group consisting of 1M>R, 1M>K, 191R>C, 344E>Q, and combinations thereof.

In some embodiments, the one or more sites of loss-of-function mutations correspond to amino acids 193 and/or 327 of a SHMT2 gene product. In some embodiments, the loss-of-function mutations are selected from the group consisting of 193R>Q, 327R>Q, and combinations thereof.

In some embodiments, the one or more sites of loss-of-function mutations correspond to amino acids 163 and/or 300 of a SLC25A32 gene product. In some embodiments, the loss-of-function mutations are selected from the group consisting of 163Y>C, 300Y>C, and combinations thereof.

In some embodiments, a first amplification step amplifies a region of a target gene. In some embodiments the amplification product is less than about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 250, 225, 200, 175 or 150 nucleotides long.

In some embodiments, processing a sample comprises genotyping a nucleic acid (e.g., an amplified nucleic acid) using techniques described herein. In some embodiments, an individual is classified as at risk of or suffering from a mitochondrial dysfunction or disorder, autism, and/or Pediatric Acute-onset Neuropsychiatric Syndrome (PANS) if they are determined by genotyping to have one or more mutant alleles. In some embodiments, mutant alleles encode an ALDH1L1, ALDH1L2, FOR1, FPGS, GCSH, GLDC, MTHFD1, MTHFD1L, MTHFD2, MTHFD2L, MTHFS, MTRR, SHMT1, SHMT2, and/or SLC25A32 mutation described herein whose presence correlates with incidence and/or risk of a mitochondrial dysfunction or disorder, autism, and/or Pediatric Acute-onset Neuropsychiatric Syndrome (PANS).

Common genotyping methods are known in the art and include, but are not limited to, sequencing, quantitative PCR, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

In some embodiments genotyping nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 comprises sequencing the amplified DNA. In some embodiments, any of a variety of sequencing reactions known in the art can be used to directly sequence at least a portion of amplified DNA. Exemplary sequencing reactions include those based on techniques developed by Maxam and Gilbert, Proc. Natl. Acad. Sci USA, 74:560 (1977) or Sanger, Proc. Nat. Acad. Sci 74:5463 (1977). It is also contemplated that any of a variety of automated sequencing procedures may be utilized when performing the subject assays, e.g., see Venter et al., Science, 291:1304-1351 (2001); Lander et al., Nature, 409:860-921 (2001), including sequencing by mass spectrometry, e.g., see U.S. Pat. No. 5,547,835 and PCT Patent Publication No. WO 94/16101 and WO 94/21822; U.S. Pat. No. 5,605,798 and PCT Patent Application No. PCT/US96/03651; Cohen et al., Adv. Chromatogr. 36:127-162 (1996); and Griffin et al., Appl. Biochem. Biotechnol. 38:147-159 (1993). It will be evident to one skilled in the art that, for some embodiments, the occurrence of only one, two or three of the nucleic acid bases need be determined in the sequencing reaction. Yet other sequencing methods are disclosed, e.g., in U.S. Pat. Nos. 5,580,732; 5,571,676; 4,863,849; 5,302,509; PCT Patent Application Nos. WO 91/06678 and WO 93/21340; Canard et al., Gene 148:1-6 (1994); Metzker et al., Nucleic Acids Research 22:4259-4267 (1994) and U.S. Pat. Nos. 5,740,341 and 6,306,597. In some embodiments, sequencing reactions comprise deep sequencing.

In some embodiments, genotyping nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25 A32 comprises hybridizing a nucleic acid detection probe to the amplified DNA, wherein the nucleic acid detection probe comprises sequence that is complimentary to the sequence of the at least one mutation. In some embodiments, hybridization of the nucleic acid detection probe to the amplified human genomic DNA is detected by quantitative PCR. “Quantitative” PCR which are also referred to as “real-time PCR” and “real-time RT-PCR,” respectively, involves detecting PCR products via a probe that provides a signal (typically a fluorescent signal) that is related to the amount of amplified product in the sample. Examples of commonly used probes used in quantitative include the following probes: TAQMAN® probes, Molecular Beacons probes, SCORPION® probes, and SYBR® Green probes. Briefly, TAQMAN® probes, Molecular Beacons, and SCORPION® probes each have a fluorescent reporter dye (also called a “fluor”) attached on or around the 5′ end of the probes and a quencher moiety attached on or around the 3′ end of the probes. In the unhybridized state, the proximity of the fluor and the quench molecules prevents the detection of fluorescent signal from the probe. During PCR, when the polymerase replicates a template on which a probe is bound, the 5′-nuclease activity of the polymerase cleaves the probe at a site between the fluor and quencher thus, increasing fluorescence with each replication cycle. SYBR® Green probes bind double-stranded DNA and upon excitation emit light; thus as PCR product accumulates, fluorescence increases.

In some embodiments, the nucleic acid detection probe detect nucleic acids that encode a 23G>D mutation of ALDH1L1. In some embodiments, the nucleic acid detection probe detect nucleic acids that encode a 1175>L mutation of ALDH1L1. In some embodiments, the nucleic acid detection probe detect nucleic acids that encode a 333R>Q mutation of ALDH1L1. In some embodiments, the nucleic acid detection probe detect nucleic acids that encode a 4485>N mutation of ALDH1L1. In some embodiments, the nucleic acid detection probe detect nucleic acids that encode a 524G>S mutation of ALDH1L1. In some embodiments, the nucleic acid detection probe detect nucleic acids that encode a 666N>K mutation of ALDH1L1. In some embodiments, the nucleic acid detection probe detect nucleic acids that encode a 760E>K mutation of ALDH1L1. In some embodiments, the nucleic acid detection probe detect nucleic acids that encode a 771T>A mutation of ALDH1L1. In some embodiments, the nucleic acid detection probe detect nucleic acids that encode a 876K>R mutation of ALDH1L1. In some embodiments, the nucleic acid detection probe detect nucleic acids that encode a frame shift p.Ala107Profs64X mutation of ALDH1L1.

In some embodiments, the nucleic acid detection probe detect nucleic acids that encode a 204L>F mutation of ALDH1L2. In some embodiments, the nucleic acid detection probe detect nucleic acids that encode a 603W>X mutation of ALDH1L2. In some embodiments, the nucleic acid detection probe detect nucleic acids that encode a 748V>A mutation of ALDH1L2. In some embodiments, the nucleic acid detection probe detect nucleic acids that encode a 796G>R mutation of ALDH1L2. In some embodiments, the nucleic acid detection probe detect nucleic acids that encode a 833T>I mutation of ALDH1L2. In some embodiments, the nucleic acid detection probe detect nucleic acids that encode a 918T>M mutation of ALDH1L2.

In some embodiments, the nucleic acid detection probe detect nucleic acids that encode a 98R>W mutation of FOLR1.

In some embodiments, the nucleic acid detection probe detect nucleic acids that encode a 50R>C mutation of FPGS. In some embodiments, the nucleic acid detection probe detect nucleic acids that encode a 85R>W mutation of FPGS. In some embodiments, the nucleic acid detection probe detect nucleic acids that encode a 162R>Q mutation of FPGS. In some embodiments, the nucleic acid detection probe detect nucleic acids that encode a 466R>C mutation of FPGS.

In some embodiments, the nucleic acid detection probe detect nucleic acids that encode a 84Y>H mutation of GCSH.

In some embodiments, the nucleic acid detection probe detect nucleic acids that encode a 18G>C mutation of GLDC. In some embodiments, the nucleic acid detection probe detect nucleic acids that encode a 147I>M mutation of GLDC. In some embodiments, the nucleic acid detection probe detect nucleic acids that encode a 503E>A mutation of GLDC. In some embodiments, the nucleic acid detection probe detect nucleic acids that encode a 675N>K mutation of GLDC. In some embodiments, the nucleic acid detection probe detect nucleic acids that encode a 705V>M mutation of GLDC. In some embodiments, the nucleic acid detection probe detect nucleic acids that encode a 716L>H mutation of GLDC. In some embodiments, the nucleic acid detection probe detect nucleic acids that encode a 895M>V mutation of GLDC. In some embodiments, the nucleic acid detection probe detect nucleic acids that encode a 937R>L mutation of GLDC. In some embodiments, the nucleic acid detection probe detect nucleic acids that encode a 966Q>H mutation of GLDC.

In some embodiments, the nucleic acid detection probe detect nucleic acids that encode a 830A>V mutation of MTHFD1.

In some embodiments, the nucleic acid detection probe detect nucleic acids that encode a 31A>G mutation of MTHFD1L. In some embodiments, the nucleic acid detection probe detect nucleic acids that encode a 520Y>C mutation of MTHFD1L. In some embodiments, the nucleic acid detection probe detect nucleic acids that encode a 564R>H mutation of MTHFD1L. In some embodiments, the nucleic acid detection probe detect nucleic acids that encode a 949G>R mutation of MTHFD1L.

In some embodiments, the nucleic acid detection probe detect nucleic acids that encode a 263D>G mutation of MTHFD2.

In some embodiments, the nucleic acid detection probe detect nucleic acids that encode a 161G>E mutation of MTHFD2L. In some embodiments, the nucleic acid detection probe detect nucleic acids that encode a 210V>L mutation of MTHFD2L.

In some embodiments, the nucleic acid detection probe detect nucleic acids that encode a 133L>Q mutation of MTHFS. In some embodiments, the nucleic acid detection probe detect nucleic acids that encode a 174E>K mutation of MTHFS.

In some embodiments, the nucleic acid detection probe detect nucleic acids that encode a 317I>T mutation of MTRR. In some embodiments, the nucleic acid detection probe detect nucleic acids that encode a 517T>A mutation of MTRR.

In some embodiments, the nucleic acid detection probe detect nucleic acids that encode a 1M>R mutation of SHMT1. In some embodiments, the nucleic acid detection probe detect nucleic acids that encode a 1M>K mutation of SHMT1. In some embodiments, the nucleic acid detection probe detect nucleic acids that encode a 191R>C mutation of SHMT1. In some embodiments, the nucleic acid detection probe detect nucleic acids that encode a 344E>Q mutation of SHMT1.

In some embodiments, the nucleic acid detection probe detect nucleic acids that encode a 193R>Q mutation of SHMT2. In some embodiments, the nucleic acid detection probe detect nucleic acids that encode a 327R>Q mutation of SHMT2.

In some embodiments, the nucleic acid detection probe detect nucleic acids that encode a 163Y>C mutation of SLC25A32. In some embodiments, the nucleic acid detection probe detect nucleic acids that encode a 300Y>C mutation of SLC25A32.

In some embodiments genotyping nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 comprises a primer extension reaction. Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. In some embodiments a primer extension reaction comprises contacting the amplified nucleic acid with one or more primers which specifically hybridize to a region of the isolated nucleic acid containing a mutation, and amplifying the hybridized amplified nucleic acid to detect the nucleotide present at the position of interest. In some embodiments detecting the presence or absence of an amplification product (assays can be designed so that hybridization and/or amplification will only occur if a particular mutation is present or absent).

Therapy

The present invention encompasses the recognition that administration of folinic acid, glycine or a pharmaceutically acceptable salt thereof, represents an effective therapy for autism, mitochondrial dysfunctions or disorders and/or Pediatric Acute-onset Neuropsychiatric Syndrome (PANS), wherein nuclear DNA of the individual that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 includes a loss-of function mutation. The present invention proposes that administration of folinic acid, glycine or a pharmaceutically acceptable salt thereof to a subject whose ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 includes a loss-of-function mutation restores folate balance, and is an effective therapy a mitochondrial dysfunction or disorder, autism, and/or Pediatric Acute-onset Neuropsychiatric Syndrome (PANS).

In some embodiments, the current invention provides methods of treating or reducing risk for a mitochondrial dysfunction or disorder, autism, and/or Pediatric Acute-onset Neuropsychiatric Syndrome (PANS) comprising administering to a subject folinic acid, glycine or a pharmaceutically acceptable salt thereof. In certain embodiments, the methods comprise administering to the individual a therapeutically effective amount of folinic acid, glycine or a pharmaceutically acceptable salt thereof, wherein nuclear DNA of the individual that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 includes a loss-of function mutation.

In some embodiments, classifying the individual as one that does or does not possess a mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 according to the methods described herein further comprises providing the individual or a physician treating the individual with information regarding the mutation. In some embodiments, the information references a correlation between the mutation and the potential benefits of therapy with folinic acid, glycine, or a pharmaceutically acceptable salt thereof.

In some embodiments, the invention described herein comprises methods of aiding in the selection of a therapy for an individual at risk of or suffering from a mitochondrial dysfunction or disorder, autism, and/or Pediatric Acute-onset Neuropsychiatric Syndrome (PANS), the method comprising obtaining a sample of nuclear DNA from the individual, processing the sample to determine whether the individual possesses a loss-of-function mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32, and classifying the individual as one that could benefit from therapy with folinic acid, glycine or a pharmaceutically acceptable salt thereof if the step of processing determines that the individual possesses a loss-of-function mutation in nuclear DNA that encodes ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 using techniques described herein.

In accordance with the methods of the invention, folinic acid, glycine or a pharmaceutically acceptable salt thereof can be administered to a subject alone, or as a component of a composition or medicament (e.g., in the manufacture of a medicament for the prevention or treatment of a mitochondrial dysfunction or disorder, autism, and/or Pediatric Acute-onset Neuropsychiatric Syndrome (PANS)), as described herein. The compositions can be formulated with a physiologically acceptable carrier or excipient to prepare a pharmaceutical composition. The carrier and composition can be sterile. The formulation should suit the mode of administration. Methods of formulating compositions are known in the art (see, e.g., Remington's Pharmaceuticals Sciences, 17^{th} Edition, Mack Publishing Co., (Alfonso R. Gennaro, editor) (1989)). Suitable pharmaceutically acceptable carriers are known in the art.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulations can include standard carriers such as pharmaceutical grades of mannitol, lactose, starch, magnesium stearate, polyvinyl pyrollidone, sodium saccharine, cellulose, magnesium carbonate, etc.

Folinic acid, glycine or a pharmaceutically acceptable salt thereof described herein (or a composition or medicament containing an agent described herein) is administered by any appropriate route. In some embodiments, folinic acid, glycine or a pharmaceutically acceptable salt thereof is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, folinic acid, glycine or a pharmaceutically acceptable salt thereof is administered intravenously. In some embodiments, folinic acid, glycine or a pharmaceutically acceptable salt thereof is administered orally. In other embodiments, folinic acid, glycine or a pharmaceutically acceptable salt thereof is administered by direct administration to a target tissue, such as heart or muscle (e.g., intramuscular), tumor (intratumorallly), nervous system (e.g., direct injection into the brain; intraventricularly; intrathecally). Alternatively, folinic acid, glycine or a pharmaceutically acceptable salt thereof (or a composition or medicament containing an agent) can be administered by inhalation, parenterally, intradermally, transdermally, or transmucosally (e.g., orally or nasally). More than one route can be used concurrently, if desired.

In various embodiments, folinic acid, glycine or a pharmaceutically acceptable salt thereof is administered at a therapeutically effective amount. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating the underlying disease or condition). In some particular embodiments, appropriate doses or amounts to be administered may be extrapolated from dose-response curves derived from in vitro or animal model test systems.

In some embodiments, a composition is administered in a therapeutically effective amount and/or according to a dosing regimen that is correlated with a particular desired outcome (e.g., with treating or reducing risk for a mitochondrial dysfunction or disorder, autism, and/or Pediatric Acute-onset Neuropsychiatric Syndrome (PANS)).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, or combinations thereof).

In some embodiments, a provided composition is provided as a pharmaceutical formulation. In some embodiments, a pharmaceutical formulation is or comprises a unit dose amount for administration in accordance with a dosing regimen correlated with achievement of the reduced incidence or risk of a mitochondrial dysfunction or disorder, autism, and/or Pediatric Acute-onset Neuropsychiatric Syndrome (PANS).

In some embodiments, a formulation comprising folinic acid, glycine or a pharmaceutically acceptable salt thereof described herein is administered as a single dose. In some embodiments, a formulation comprising folinic acid, glycine or a pharmaceutically acceptable salt thereof described herein is administered at regular intervals. Administration at an “interval,” as used herein, indicates that the therapeutically effective amount is administered periodically (as distinguished from a one-time dose).

In some embodiments, a formulation comprising folinic acid, glycine or a pharmaceutically acceptable salt thereof described herein is administered at regular intervals indefinitely. In some embodiments, a formulation comprising folinic acid, glycine or a pharmaceutically acceptable salt thereof described herein is administered at regular intervals for a defined period.

Kits

In some embodiments, the present invention provides kits comprising materials useful for the amplification and detection or sequencing of the nuclear DNA that encompasses part or all of the ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 gene product according to methods described herein. The inventive kits may be used by diagnostic laboratories, experimental laboratories, or practitioners. In some embodiments, the present disclosure provides kits further comprising materials useful for treating a mitochondrial dysfunction or disorder, autism, and/or Pediatric Acute-onset Neuropsychiatric Syndrome (PANS). In some embodiments, the materials useful for treating the mitochondrial dysfunction or disorder, autism, and/or Pediatric Acute-onset Neuropsychiatric Syndrome (PANS) are folinic acid, glycine or a pharmaceutically acceptable salt thereof.

Materials and reagents useful for the detection or sequencing of the nuclear DNA that encompasses part or all of the ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 gene products according to the present disclosure may be assembled together in a kit. In some embodiments, an inventive kit comprises at least one inventive primer set, and optionally, amplification reaction reagents. In some embodiments, a kit comprises reagents which render the procedure specific. In some embodiments, the kit comprises nucleic detection probes. Thus, a kit intended to be used for the detection of a particular loss-of-function mutation preferably comprises primer sets and/or probes described herein that can be used to amplify and/or detect a particular ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 target sequence of interest. A kit intended to be used for the multiplex detection of a plurality of ALDH1L1, ALDH1L2, FOLR1, FPGS, GLDC, MTHFD1, MTHFD1L, MTHFD2, MTHFD2L, MTHFS, MTRR, SHMT1, SHMT2, and/or SLC25A32 target preferably comprises a plurality of primer sets and/or probes (optionally in separate containers) described herein that can be used to amplify and/or detect ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 target sequences described herein.

Suitable amplification reaction reagents that can be included in an inventive kit include, for example, one or more of: buffers; enzymes having polymerase activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and deoxythymidine triphosphate, biotinylated dNTPs, suitable for carrying out the amplification reactions.

Depending on the procedure, the kit may further comprise one or more of: wash buffers and/or reagents, hybridization buffers and/or reagents, labeling buffers and/or reagents, and detection means. The buffers and/or reagents included in a kit are preferably optimized for the particular amplification/detection technique for which the kit is intended. Protocols for using these buffers and reagents for performing different steps of the procedure may also be included in the kit.

Furthermore, the kits may be provided with an internal control as a check on the amplification procedure and to prevent occurrence of false negative test results due to failures in the amplification procedure. An optimal control sequence is selected in such a way that it will not compete with the target nucleic acid sequence in the amplification reaction (as described above).

Kits may also contain reagents for the isolation of nucleic acids from biological specimen prior to amplification.

The reagents may be supplied in a solid (e.g., lyophilized) or liquid form. The kits of the present disclosure optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents, primer sets, primer/probe sets and/or folinic acid, glycine or a pharmaceutically acceptable salt thereof according to the present disclosure. Instructions for using the kit according to one or more methods of the present disclosure may comprise instructions for processing the biological sample, extracting nucleic acid molecules, and/or performing the test; instructions for interpreting the results as well as a notice in the form prescribed by a governmental agency (e.g., FDA) regulating the manufacture, use or sale of pharmaceuticals or biological products.

Computer Systems

Methods described herein can be implemented in a computer system having a processor that executes specific instructions in a computer program. The computer system may be arranged to output a medication profile based on receiving an individual's genotype (e.g., AALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 polymorphism(s)). Particularly, the computer program may include instructions for the system to select the most appropriate medication (e.g., folinic acid, glycine) for an individual.

In some embodiments, the computer program may be configured such that the computer system can identify the genotype based on received data and provide a preliminary identification of the universe of possible medications. The system may be able to rank-order the identified medications based on specific co-factors in the algorithmic equation. The system may be able to adjust the rank ordering based on the genotypic polymorphism(s) carried by the individual. The system may be able to adjust the rank ordering based on clinical responses, such as by family members of the individual.

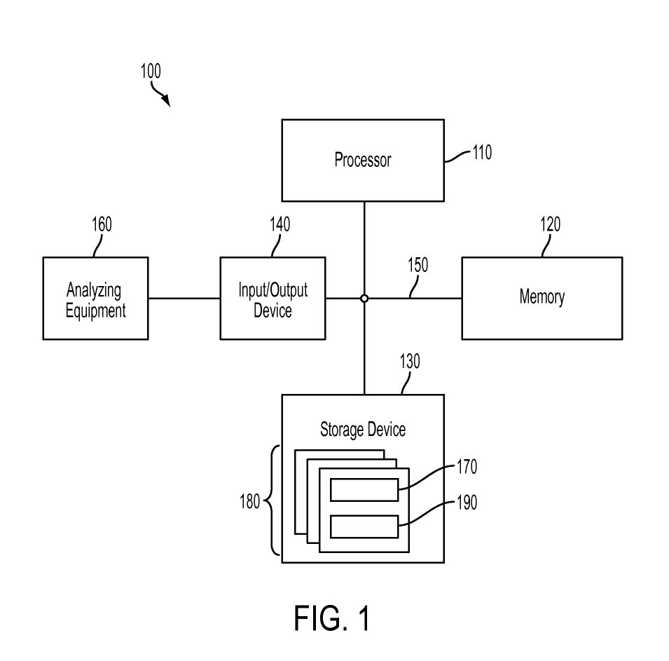

FIG. 1 is a block diagram of a computer system 100 that can be used in the operations described above, according to one embodiment. The system 100 includes a processor 110, a memory 120, a storage device 130 and an input/output device 140. Each of the components 110, 120, 130 and 140 are interconnected using a system bus 150. The system may include analyzing equipment 160 for determining the individual's genotype.

The processor 110 is capable of processing instructions for execution within the system 100. In one embodiment, the processor 110 is a single-threaded processor. In another embodiment, the processor 110 is a multi-threaded processor. The processor 110 is capable of processing instructions stored in the memory 120 or on the storage device 130, including for receiving or sending information through the input/output device 140.

The memory 120 stores information within the system 100. In one embodiment, the memory 120 is a computer-readable medium. In one embodiment, the memory 120 is a volatile memory unit. In another embodiment, the memory 120 is a non-volatile memory unit.

The storage device 130 is capable of providing mass storage for the system 100. In one embodiment, the storage device 130 is a computer-readable medium. In various different embodiments, the storage device 130 may be a floppy disk device, a hard disk device, an optical disk device, or a tape device.

The input/output device 140 provides input/output operations for the system 100. In one embodiment, the input/output device 140 includes a keyboard and/or pointing device. In one embodiment, the input/output device 140 includes a display unit for displaying graphical user interfaces.

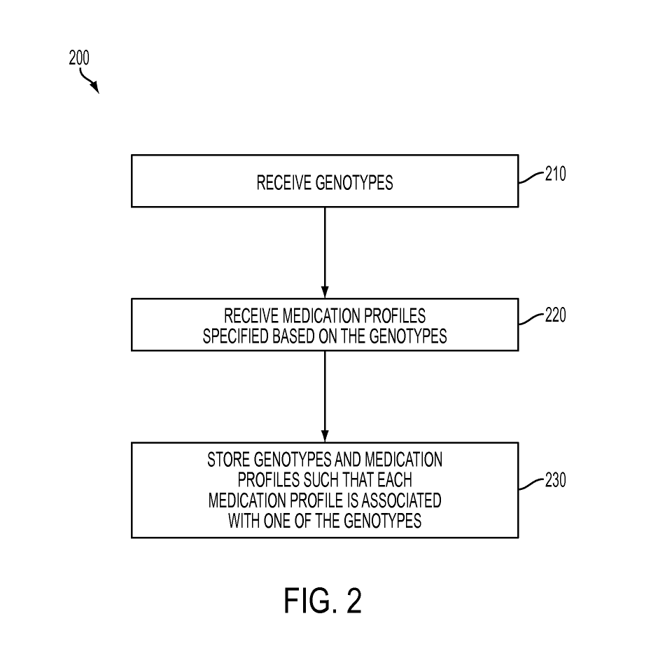

The system 100 can be used to build a database. FIG. 2 shows a flow chart of a method 200 for building a database for use in selecting a medication for an individual. Preferably, the method 200 is performed in the system 100. For example, a computer program product can include instructions that cause the processor 110 to perform the steps of the method 200. The method 200 includes the following steps.

Receiving, in step 210, a plurality of genotypes 170 for ALD1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32. A computer program in the system 100 may include instructions for presenting a suitable graphical user interface on input/output device 140, and the graphical user interface may prompt the user to enter the genotypes 170 using the input/output device 140, such as a keyboard.

Receiving, in step 220, a plurality of medication profiles 180. The medication profiles 180 are specified based on the genotypes 170. The user may enter the medication profiles 180 using the input/output device 140, such as a keyboard. For example, the medication profile 180 may include information 190 regarding at least one medication.

Storing, in step 230, the received genotypes 170 and the medication profiles 180 such that each medication profile 180 is associated with one of the genotypes 170. The system 100 may store the medication profiles 180 and the genotypes 170 in the storage device 130. For example, when the storing is complete, the system 100 can identity a particular one of the medication profiles 180 that is associated with a specific genotype 170. Having identified the medication profile 180, the system 100 can access the information 190 contained within the identified medication profile 180, as will be described in the following example.

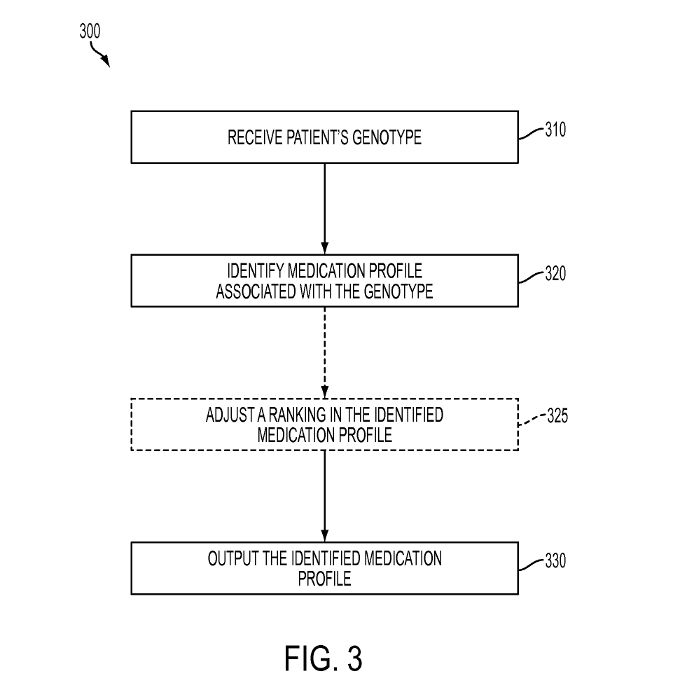

The system 100 may be used for selecting a medication. FIG. 3 shows a flow chart of a method 300 of selecting a medication for an individual. Preferably, the method 300 is performed in the system 100. For example, a computer program product can include instructions that cause the processor 110 to perform the steps of the method 300. The method 300 includes the following steps.

Receiving, in step 310, an individual's genotype for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32. The genotype may be entered by a user via input/output device 140. For example, the user may obtain the individual's genotype for ALDH1L1, ALDH1L2, FOLR1, FPGS, GCSH, GLDC, MTHFD1, MTHFD1L, MTHFD2, MTHFD2L, MTHFS, MTRR, SHMT1, SHMT2, and/or SLC25A32 using the analyzing equipment 160 (which may or may not be connected to the system 100). The user may type the individual's genotype on input/output device 140, such as a keyboard, for receipt by the system 100.

The genotype may be received directly from the analyzing equipment 160. For example, analyzing equipment 160 may include a processor and suitable software such that it can communicate over a network. The system 100 may be connected to the analyzing equipment 160 through input/output device 140, such as a network adapter, and directly receive the individual's genotype.

Identifying, in step 320, one of the medication profiles 180 that is associated with the individual's genotype. For example, the system 100 may perform a database search in the storage device 130. Particularly, the system 100 may access the genotype 170 for individual medication profiles 180 until a match is found. Optional step 325 will be described below.

Outputting, in step 330, the identified medication profile 180 in response to receiving the individual's genotype. The system may output the identified medication profile 180 through input/output device 140. For example, the identified medication profile may be printed or displayed in a suitable graphical user interface on a display device. As another example, the system 100 may transmit the identified medication profile over a network, such as a local area network or the Internet, to which the input/output device 140 is connected.

The medication profiles 180 can be created such that there is flexibility in how the system 100 outputs them. For example, the information 190 in one or more of the medication profiles 180 may include a ranking of several medications. The program may include instructions for applying rules to the received individual's genotype and adjust the ranking accordingly. In such implementations, the method 300 may include optional step 325 of adjusting the ranking before outputting the identified medication profile. For example, the system 100 may receive a genotypic polymorphism carried by the individual (optionally in the same way the individual's genotype was received) and adjust the ranking accordingly in step 325. As another example, step 325 may involve adjusting the ranking based on a clinical response. The clinical response may be received by the system 100 in the same way as the individual's genotype. For example, the ranking can be adjusted based on a clinical response by a member of the individual's family.

The medication profiles 180 may be updated as necessary. For example, the introduction of a new medication on the market may prompt a revision of one or more existing medication profiles. A new medication may also be the basis for creating a new medication profile. The adjustment or creation of medication profiles may be done substantially as described above.

The medication profiles 180 may be used for medication selection in the same system where they were created, or in a different system. That is, the system 100 may first be used for building a database of the medication profiles 180, and the system 100 may thereafter be used to select a medication profile for the genotype of a specific individual. As another example, one or more medication profiles 180 may be transmitted within a computer readable medium such as a global computer network for remote processing according to the invention.

### Exemplification

### EXAMPLE 1

### Novel Disease Associations and Novel Disease-Associated Genes

With the advent of NextGen DNA sequencing in the diagnosis of mitochondrial disease, has come the realization that many patients do not have a clear diagnosis. Perhaps the most likely explanation is that many cases are due to polygenic/multifactorial pathogenesis, as is the case in most fields of medicine. To elucidate novel associations, post-testing data analysis is key. Comprehensive sequencing of numerous nuclear genes was performed in unrelated patients with a clinical suspicion of possible mitochondrial disease. To limit type II errors due to multiple comparisons, candidates were first assigned based on an increased prevalence of deleterious-predicted variants among patients in comparison to prevalence rates from a dataset of genomes and/or in-house negative controls. Second, the phenotype of those carrying the variant(s) were compared to the phenotypes in a “referral group” of randomly-selected patients. Some of the identified genes were not previously associated with disease.

### EXAMPLE 2

### Clinical Manifestations of Folate Pathway Variants

Comprehensive sequencing of numerous nuclear genes was performed in unrelated patients with a clinical suspicion of possible mitochondrial disease and identified candidate genes with variants in the Folate Pathway.

Results are shown in Table 2 and Table 3. Table 4 shows an evolutionary assessment of each folate pathway variant, indicating the number of alignments out of those tested that matched, and how far back in the evolutionary tree the variant was found. Also indicated in Table 4 are the prevalence of the variant in the population, and an assessment of protein function with the indicated mutation.

TABLE 2
Clinical Symptoms of Folate Pathway Variants
ALDH1L1 (SEQ ID NO: 1)
Patient

| ID | Variant | Phenotype |
|---|---|---|
| 10330 | G23D | Leighs |
| 10476 | N666K | 22-year-old male with progressive seizures, sleep disorder, |
| | | autism, depression/bipolar, OCD, migraine, anxiety, |
| | | chronic fatigue and severe immunodeficiency. |
| 10551 | T771A | no clinical information |
| 10952 | T771A | severe irritability, hypersensitivity, growth issue, |
| | | twin also affected, severe PANS, immunodeficiency |
| 11150 | R333Q | autism, macrocephaly, (and) PANS |
| 11464 | p.A1a107Profs64X | Tics, OCD |
| 11551 | S448 | Ndevelopmental delay, anxiety/panic, migraines, and |
| | | congenital nystagmus |
| 11573 | G23D | severe primordial growth retardation, in-utero stroke, and |
| | | functional disease |
| 11731 | G23D | seizures, hypotonia, large bowel dysmotility and optic |
| | | neuropathy |
| 11785 | G524S | mixed seizure disorder |
| 11857 | S117L | cyclic vomiting |
| 11859 | K876R | tic disorder and transient OCD |
| 12206 | E760K | autistic spectrum disorder and a history of the arrest of |
| | | speech development |

(ALDH1L1 Negative control)

| | | |
|---|---|---|
| 10214 | G23D | Negative control (no clinical phenotype) |
| 11269 | R333Q | Negative control (no clinical phenotype) |

ALDHIL2 (SEQ ID NO: 3)

| Patient | | |
|---|---|---|
| ID | Variant | Phenotype |
| 10512 | T918M | encephalopathy (seizure disorder, mental retardation, and |
| | | cerebral palsy), optic atrophy, hearing loss, GI dysmotility |
| | | and dysautonomia |
| 10952T | 918M | severe irritability, hypersensitivity, growth issue, twin |
| | | also affected, severe PANS, |
| 11426W | 603X | Chronic severe migraine Easy fatiguability Chronic variable |
| | | immunodeficiency Hypersomnia Fibromyalgia Restless leg |
| | | syndrome |
| 11573G | 796R | severe primordial growth retardation, in-utero stroke, and |
| | | functional disease |
| 11653V | 748A | ncephalopathy that might be progressive, including nystagia, |
| | | hypotonia, abnormal movements, optic neuropathy, |
| | | strabismus, skeletal muscle weakness, and developmental |
| | | delay. |
| 11727T | 918M | apraxia and language processing disorder. He has had |
| | | episodes of regression, slurring of speech for periods of |
| | | time, recurrent illnesses and one or two seizures |
| 11833L | 204F | encephalopathy (global delay, hyptonia, anxiety disorder), |
| | | muscle fatigue/poor endurance, and GERD/chronic |
| | | respiratory problems |
| 11853W | 603X | obsessive compulsive disorder |
| 11904T | 833I | obsessive compulsive disorder and tic disorder |

(ALDH1L2 Negative control)

| | | |
|---|---|---|
| 10207V | 486A | Negative control (no clinical phenotype) |
| 11270F | 893L | Negative control (no clinical phenotype) |

FOLR1 (SEQ ID NO: 5)

| Patient | | |
|---|---|---|
| ID | Variant | Phenotype |
| 11864 | R98W | tic disorder, OCD |

FPGS (SEQ ID NO: 7)

| Patient | | |
|---|---|---|
| ID | Variant | Phenotype |
| 10171 | R466C | 7-year-old male with an acute episode of liver failure |
| | | associated with viral illness and vaccination |
| 10525 | R466C | sudden-onset OCD, progressive anxiety to the point of no |
| | | longer being able to speak or walk, conversion disorder |
| 10641 | R50C | multiple functional symptoms including migraine, chronic |
| | | fatigue, depression, postural hypotension, frequent |
| | | diarrhea, and inappropriate sweating |
| 10884 | R466C | Multiple fainting incidences, cataplexy vocal tics, lyme |
| | | disease, chronic fatigue, endocrine disorder, hyperthyroid |
| 10977 | R466C | autism, PANS, arachnoid cyst, cardiomyopathy |
| 11172 | R85W | myopathy with muscle biopsy suggestive of mitochondrial |
| | | myopathy |
| 11432 | R466C | Autism spectrum disorder, developmental delay, Lyme |
| | | disease, cerebral folate deficiency, severe gastrointestinal |
| | | distress |
| 11464 | R466C | Tics, OCD |
| 11573 | R162Q | severe primordial growth retardation, in-utero stroke, and |
| | | functional disease |
| 11573 | R466C | |
| 11609 | R466C | tic disorder and transient OCD |
| 11781 | R466C | seizures, developmental delay, colonic dysmotility, and |
| | | elevated transaminases |
| 11821 | R85W | tethered cord, who also had skeletal muscle weakness that led |
| | | to biopsy showing RRF and mitochondrial proliferation |

(FPGS Negative control)

| | | |
|---|---|---|
| 10580 | R85W | Negative control (no clinical phenotype) |

GCSH (SEQ ID NO: 9)

| Patient | | |
|---|---|---|
| ID | Variant | Phenotype |
| 10647 | Y84H | cyclic vomiting syndrome |

GLDC (SEQ ID NO: 11)

| Patient | | |
|---|---|---|
| ID | Variant | Phenotype |
| 10197 | N675K | |
| 10482 | G18C | developmental delay, hypotonia, and skeletal muscle |
| | | weakness |
| 10507 | I147M | functional/dysautonomic/neurological disease, including |
| | | peripheral neuropathy, migraine, POTS/syncope, myalgia, |
| | | chronic fatigue, and anxiety/panic |
| 10512 | V705M | encephalopathy (seizure disorder, mental retardation, and |
| | | cerebral palsy), optic atrophy, hearing loss, GI dysmotility |
| | | and dysautonomia |
| 10570 | V705M | autism |
| 11150 | V705M | autism, macrocephaly, (and) PANS |
| 11156 | R937L | episodes of catamenial cyclic vomiting syndrome |
| 11712 | Q966H | seizures, hypotonia, ataxia, spasticity, skeletal muscle |
| | | weakness, large bowel dysmotility, and developmental delay |
| 11765 | M895V | multiple functional/dysautonomic symptomatology, including |
| | | chronic pain and post-prandial nausea |
| 11791 | M895V | OCD and Streptococcus group A |
| 11855 | Q966H | obsessive compulsive disorder and a transient tic disorder |
| 11887 | L716H | OCD, a tic disorder |
| 11904 | V705 | Mobsessive compulsive disorder and tic disorder |
| 12049 | E503 | tachycardia, pancreatitis, growth retardation, large bowel |
| | | Adisease, chronic fatigue, developmental delay, and skeletal |
| | | muscle weakness |
| 12120 | G18C | cyclic vomiting since age 1 year, migraine and constipation |

(GLDC Negative control)

| | | |
|---|---|---|
| 12149 | E503A | Negative control (no clinical phenotype) |

MTHFD1 (SEQ ID NO: 13)

| Patient | | |
|---|---|---|
| ID | Variant | Phenotype |
| 11968 | A830V | encephalopathy, paresthesia, OCD, anxiety/panic |

(MTHFD1 Negative control)

| | | |
|---|---|---|
| 10623 | G734A | Negative control (no clinical phenotype) |

MTHFD1L (SEQ ID NO: 15)

| Patient | | |
|---|---|---|
| ID | Variant | Phenotype |
| 10345 | A31G | autistic spectrum disorder, developmental delay, growth |
| | | retardation, decreased muscle mass and constipation |
| 10651 | G949R | |
| 10937 | A31G | intractable seizures, movement disorder and severe |
| | | developmental delays who is G-tube dependent and has |
| | | constipation |
| 11245 | R564H | psychosis (currently hospitalized for schizophrenia) |
| | | depression, anxiety/panic, OCD, and Marfanoid habitus |
| 11247 | R564H | immunodeficiency, an extremely-high IgE, an autistic |
| | | spectrum disorder and PANS |
| 11434 | Y520C | Down syndrome, severe OCD, progressive dementia, autism, |
| | | sleep disorder, and migraines |
| 11571 | R564H | Encephalopathy, including cognitive impairment, chorea, |
| | | dystonia (improved on a ketogenic diet), and seizures |
| | | (frontal lobe and deep |
| 11658 | R564H | |
| 11662 | R564H | to 3 months of behavioral deterioration with auditory/visual |
| | | hallucinations, past history language disorder, ADD, and a |
| | | strong paternal family history severe psychiatric disorders |
| 11731 | R564H | seizures, hypotonia, large bowel dysmotility and optic |
| | | neuropathy |
| 11857 | R564H | cyclic vomiting syndrome in which the predominate symptom |
| | | is dizziness, |
| 12009 | R564H | tic disorder, streptococcus group A, mycoplasma, and |
| | | obsessive compulsive disorder |

(MTHFD1L Negative control)

| | | |
|---|---|---|
| 11259 | R564H | Negative control (no clinical phenotype) |
| 11267 | R564H | Negative control (no clinical phenotype) |

MTHFD2 (SEQ ID NO: 17)

| Patient | | |
|---|---|---|
| ID | Variant | Phenotype |
| 10599 | D263G | sudden-onset OCD, motor tics, and IgA deficiency |

MTHFD2L (SEQ ID NO: 19)

| Patient | | |
|---|---|---|
| ID | Variant | Phenotype |
| 11172 | G161E | myopathy with muscle biopsy suggestive of mitochondrial |
| | | myopathy |
| 11312 | V210L | autistic spectrum and obsessive compulsive disorders |
| 11347 | G161E | autism |

MTHFS (SEQ ID NO: 21)

| Patient | | |
|---|---|---|
| ID | Variant | Phenotype |
| 10163 | L133Q | |
| 10342 | L133Q | |
| 10343 | L133Q | |
| 11904 | E174K | obsessive compulsive disorder and tic disorder |

MTRR (SEQ ID NO: 23)

| Patient | | |
|---|---|---|
| ID | Variant | Phenotype |
| 10482 | 1317T | developmental delay, hypotonia, and skeletal muscle |
| | | weakness |
| 10599 | T517A | Sudden-onset OCD, motor tics, and IgA deficiency |

(MTRR Negative control)

| | | |
|---|---|---|
| 11949 | V634I | Negative control (no clinical phenotype) |

SHMT1 (SEQ ID NO: 25)

| Patient | | |
|---|---|---|
| ID | Variant | Phenotype |
| 10570 | R191C | autism |
| 11101 | E344Q (homo) | loss of milestones |
| 11344 | M1R | autism, developmental delay, speech apraxia, and seizures |
| 11917 | M1K | autistic-like behaviors, hypotonia, apraxia, visual |
| | | processing disorder and learning disabilities |
| 12009 | M1K | tic disorder, streptococcus group A, mycoplasma, and |
| | | obsessive compulsive disorder |

SHMT2 (SEQ ID NO: 27)

| Patient | | |
|---|---|---|
| ID | Variant | Phenotype |
| 10482 | R193Q | developmental delay, hypotonia, and skeletal muscle |
| | | weakness |
| 11772 | R327Q | partial seizures, intention tremor, daytime sleepiness, |
| | | muscle fatigue, headache, intermittent aphasia, acute visual |
| | | decline, progressive clumsiness, and precocious puberty |

(SHMT2 Negative control)

| | | |
|---|---|---|
| 10621 | R100C | Negative control (no clinical phenotype) |

SLC25A32 (SEQ ID NO: 29)

| Patient | Variant | Phenotype |
|---|---|---|
| 11765 | Y300C | functional/dysautonomic symptomatology, including chronic |
| | | pain and post-prandial nausea |
| 11816 | Y163C | OCD |

TABLE 3
Individuals with multiple folate pathway variants

| Patient | | | | | | |
|---|---|---|---|---|---|---|
| ID | Gene and Variant | | Gene and Variant | | Gene and Variant | |
| 11573 | ALDH1L1 | G23D | ALDH1L2 | G796R | FPGS | R162Q |
| | | | | | (x2) | and |
| | | | | | | R466C |
| 10952 | ALDH1L1 | T771A | ALDH1L2 | T918M | | |
| 11464 | ALDH1L1 | p.Ala107Profs64X | FPGS | R466C | | |
| | | (frame shift) | | | | |
| 11150 | ALDH1L1 | R333Q | GLDC | V705M | | |
| 11731 | ALDH1L1 | G23D | MTHFD1L | R564H | | |
| 11857 | ALDH1L1 | S117L | MTHFD1L | R564H | | |
| 11904 | ALDH1L2 | T833I | GLDC | V705M | MTHFS | E174K |
| 10512 | ALDH1L2 | T918M | GLDC | V705M | | |
| 11172 | FPGS | R85W | MTHFD2L | G161E | | |
| 10482 | GLDC | G18C | MTRR | I317T | SHMT2 | R193Q |
| 10570 | GLDC | V705M | SHMT1 | R191C | | |
| 11765 | GLDC | M895V | SLC25A32 | Y300C | | |
| 12009 | MTHFD1L | R564H | SHMT1 | M1K | | |
| 10599 | MTHFD2 | D263G | MTRR | T517A | | |

TABLE 4
Prevalence and evolutionary assessment of Folate Pathway variants
ALDH1L1 (SEQ ID NO: 1)

| Patient | | | Protein | |
|---|---|---|---|---|
| ID | Variant | Evolutionary conserved | function | Prevalence |
| 10330 | G23D | 36/36-Lamprey | O/O/Y/O | 0% |
| 10476 | N666K | 31/31-Lamprey | O/O/Y/O | 0% |
| 10551 | T771A | 26/30-Lamprey | Y/G/Y/O | 0% |
| 10952 | T771A | 26/30-Lamprey | Y/G/Y/O | 0% |
| 11150 | R333Q | 28/29 vertebrates through | Y/Y/G/Y | 0.61% |
| | | Xenopus | | |
| 11464 | p.A1a107Profs64X | Frame shift | | |
| 11551 | S448N | 24/33-Lamprey | G/G/G/Y | 0.32% |
| 11573 | G23D | 36/36-Lamprey | O/O/Y/O | 0% |
| 11731 | G23D | 36/36-Lamprey | O/O/Y/O | 0% |
| 11785 | G524S | 31/35-Lamprey | O/O/G/O | 0% |
| 11857 | S117L | 35/35-Lamprey | O/O/Y | 0% |
| 11859 | K876R | 34/34-Lamprey | O/O/Y/O | 0% |
| 12206 | E760K | 30/30-Lamprey | O/Y/G/O | 0% |

(ALDH1L1 Negative control)

| | | | | |
|---|---|---|---|---|
| 10214 | G23D | 36/36-Lamprey | O/O/Y/O | 0% |
| 11269 | R333Q | 28/29 vertebrates through | Y/Y/G/Y | 0.61% |
| | | Xenopus | | |

ALDH1L2 (SEQ ID NO: 3)

| Patient | | | Protein | |
|---|---|---|---|---|
| ID | Variant | Evolutionary conserved | function | Prevalence |
| 10512 | T918M | T or A in 41/41-zFish | G/O/Y/O | 0% |
| 10952 | T918M | T or A in 41/41-zFish | G/O/Y/O | 0% |
| 11426 | W603X | | | 0% |
| 11573 | G796R | 43/43-Lamprey | O/O/O/O | 0% |
| 11653 | V748A | 40/41-Lamprey | O/O/G/Y | 0% |
| 11727 | T918M | T or A in 41/41-zFish | G/O/Y/O | 0% |
| 11833 | L204F | 42/43-Z fish | O/G/Y/O | 0% |
| 11853 | W603X | 0% | | |
| 11904 | T833I | 39/39-Lamprey | O/O/Y/O | 0% |

(ALDH1L2 Negative control)

| | | | | |
|---|---|---|---|---|
| 10207 | V486A | 44/44-Lamprey | O/O/Y/O | 0% |
| 11270 | F893L | 40/40-Lamprey | O/O/Y/O | 0% |

FOLR1 (SEQ ID NO: 5)

| Patient | | | Protein | |
|---|---|---|---|---|
| ID | Variant | Evolutionary conserved | function | Prevalence |
| 11864 | R98W | R or K 31/38-Lamprey | G/O/Y/Y | 0.42% |

FPGS (SEQ ID NO: 7)

| Patient | | | Protein | |
|---|---|---|---|---|
| ID | Variant | Evolutionary conserved | function | Prevalence |
| 10171 | R466C | 35/35-Zfish | O/O/Y/O | 0.52% |
| 10525 | R466C | 35/35-Zfish | O/O/Y/O | 0.52% |
| 10641 | R50C | 30/30-Xenopus; C in fish | O/G/G/Y | 0% |
| 10884 | R466C | 35/35-Zfish | O/O/Y/O | 0.52% |
| 10977 | R466C | 35/35-Zfish | O/O/Y/O | 0.52% |
| 11172 | R85W | 33/34-Zfish | O/O/O/O | 0.72% |
| 11432 | R466C | 35/35-Zfish | O/O/Y/O | 0.52% |
| 11464 | R466C | 35/35-Zfish | O/O/Y/O | 0.52% |
| 11573 | R162Q | 34/38-Lamprey | Y/G/G/Y | 0% |
| 11573 | R466C | 35/35-Zfish | O/O/Y/O | 0.52% |
| 11609 | R466C | 35/35-Zfish | O/O/Y/O | 0.52% |
| 11781 | R466C | 35/35-Zfish | O/O/Y/O | 0.52% |
| 11821 | R85W | 33/34-Zfish | O/O/O/O | 0.72% |

(FPGS Negative control)

| | | | | |
|---|---|---|---|---|
| 10580 | R85W | 33/34-Zfish | O/O/O/O | 0.72% |

GCSH (SEQ ID NO: 9)

| Patient | | | Protein | |
|---|---|---|---|---|
| ID | Variant | Evolutionary conserved | function | Prevalence |
| 10647 | Y84H | 40/40-Lamprey | O/O/Y/O | 0% |

GLDC (SEQ ID NO: 11)

| Patient | | | Protein | |
|---|---|---|---|---|
| ID | Variant | Evolutionary conserved | function | Prevalence |
| 10197 | N675K | N or S-43/43-Zfish | O/G/Y/O | 0% |
| 10482 | G18C | 30/33-Medaka | G/Y/G/Y | 0.36% |
| 10507 | I147M | 42/43-Lamprey | O/O/O/O | 0% |
| 10512 | V705M | 40/41-Zfish | O/G/Y/O | 0.82% |
| 10570 | V705M | 40/41-Zfish | O/G/Y/O | 0.82% |
| 11150 | V705M | 40/41-Zfish | O/G/Y/O | 0.82% |
| 11156 | R937L | 40/40-Zfish | O/O/Y/Y | 0% |
| 11712 | Q966H | 40/40-Zfish | O/O/Y/O | 0% |
| 11765 | M895V | 39/39-Zfish | O/G/G/Y | 0% |
| 11791 | M895V | 39/39-Zfish | O/G/G/Y | 0% |
| 11855 | Q966H | 40/40-Zfish | O/O/Y/O | 0% |
| 11887 | L716H | 41/41-Zfish | O/O/Y/O | 0% |
| 11904 | V705M | 40/41-Zfish | O/G/Y/O | 0.82% |
| 12049 | E503A | 37/38-Zfish | O/G/G/G | 0% |
| 12120 | G18C | 30/33-Medaka | G/Y/G/Y | 0.36% |

(GLDC Negative control)

| | | | | |
|---|---|---|---|---|
| 12149 | E503A | 37/38-Zfish | O/G/G/G | 0% |

MTHFD1 (SEQ ID NO: 13)

| Patient | | | Protein | |
|---|---|---|---|---|
| ID | Variant | Evolutionary conserved | function | Prevalence |
| 11968 | A830V | 38/38-lamprey | O/O/Y/G | 0% |

(MTHFD1 Negative control)

| | | | | |
|---|---|---|---|---|
| 10623 | G734A | 43/43-Lamprey | O/O/O/Y | 0% |

MTHFD1L (SEQ ID NO: 15)

| Patient | | | Protein | |
|---|---|---|---|---|
| ID | Variant | Evolutionary conserved | function | Prevalence |
| 10345 | A31G | 12/16-Lamprey | G/G/G/O | 0% |
| 10651 | G949R | 42/42-Lamprey | O/O/O/O | 0% |
| 10937 | A31G | 12/16-Lamprey | G/G/G/O | 0% |
| 11245 | R564H | 40/40-Zfish | Y/O/O/O | 1.05% |
| 11247 | R564H | 40/40-Zfish | Y/O/O/O | 1.05% |
| 11434 | Y520C | 44/44-Lamprey | O/O/O/O | 0% |
| 11571 | R564H | 40/40-Zfish | Y/O/O/O | 1.05% |
| 11658 | R564H | 40/40-Zfish | Y/O/o/0 | 1.05% |
| 11662 | R564H | 40/40-Zfish | Y/O/O/O | 1.05% |
| 11731 | R564H | 40/40-Zfish | Y/O/O/O | 1.05% |
| 11857 | R564H | 40/40-Zfish | Y/O/O/O | 1.05% |
| 12009 | R564H | 40/40-Zfish | Y/O/o/O | 1.05% |

(MTHFD1L Negative control)

| | | | | |
|---|---|---|---|---|
| 11259 | R564H | 40/40-Zfish | Y/O/O/O | 1.05% |
| 11267 | R564H | 40/40-Zfish | Y/O/O/O | 1.05% |

MTHFD2 (SEQ ID NO: 17)

| Patient | | | Protein | |
|---|---|---|---|---|
| ID | Variant | Evolutionary conserved | function | Prevalence |
| 10599 | D263G | 37/37-Zfish | O/O/Y/O | 0% |

MTHFD2L (SEO ID NO: 19)

| Patient | | | Protein | |
|---|---|---|---|---|
| ID | Variant | Evolutionary conserved | function | Prevalence |
| 11172 | G161E | G or A in 38/38-Zfish | G | 0% |
| 11312 | V210L | 39/39-Zfish | Y | 0% |
| 11347 | G161E | G or A in 38/38-Zfish | G | 0% |

MTHFS (SEQ ID NO: 21)

| Patient | | | Protein | |
|---|---|---|---|---|
| ID | Variant | Evolutionary conserved | function | Prevalence |
| 10163 | L133Q | 42/43-Zfish | O/G/O/O | 0% |
| 10342 | L133Q | 42/43-Zfish | O/G/O/O | 0% |
| 10343 | L133Q | 42/43-Zfish | O/G/O/O | 0% |
| 11904 | E174K | 38/42-Zfish, D in shrew | O/G/Y/Y | 0% |
| | | and lizard, A in Xenopus, | | |
| | | and V in squirrel | | |

MTRR (SEQ ID NO: 23)

| Patient | | | Protein | |
|---|---|---|---|---|
| ID | Variant | Evolutionary conserved | function | Prevalence |
| 10482 | 1317T | I or V in 41/42-Lamprey | G/G/G/G | 0% |
| 10599 | T517A | 36/36-Lamprey | O/O/Y/O | 0.56% |

(MTRR Negative control)

| | | | | |
|---|---|---|---|---|
| 11949 | V634I | 38/40-Lamprey | O/Y/Y/O | 0.56% |

SHMT1 (SEQ ID NO: 25)

| Patient | | | Protein | |
|---|---|---|---|---|
| ID | Variant | Evolutionary conserved | function | Prevalence |
| 10570 | R191C | 39/43-Lamprey | O/O/Y/O | 0% |
| 11101 | E344Q (homo) | 32/43 Vertebrates, Dolphin | O/G/G/Y | 0% |
| | | & Horse (A), Cow, Lizard | | |
| | | & Medaka (G), 4 Fish (D) | | |
| | | and Lamprey (H) | | |
| 11344 | M1R | 33/33-Z fish | G/O/O | 0% |
| 11917 | M1K | 33/33-Z fish | G/O/O | 0.15% |
| 12009 | M1K | 33/33-Z fish | G/O/O | 0.15% |

SHMT2 (SEQ ID NO: 27)

| Patient | | | Protein | |
|---|---|---|---|---|
| ID | Variant | Evolutionary conserved | function | Prevalence |
| 10482 | R193Q | R or K in 39/39-Lamprey | O/Y/G/G | 0% |
| 11772 | R327Q | R or K in 34/34-Zfish | O/Y/G/O | 0% |

(SHMT2 Negative control)

| | | | | |
|---|---|---|---|---|
| 10621 | R100C | R or K in 35/38-Lamprey | O/Y/G | 0% |

SLC25A32 (SEQ ID NO: 29)

| Patient | | | Protein | |
|---|---|---|---|---|
| ID | Variant | Evolutionary conserved | function | Prevalence |
| 11765 | Y300C | 39/40-Zfish | O/O/O/O | 1.20% |
| 11816 | Y163C | 41/42-Zfish | O/O/Y/O | 0.09% |

Note: Severity of damaging mutations was measured by Mutation Taster (www.softgenetics.com/mutationSurveyor.html), PolyPhen (genetics.bwh.harvard.edu/pph2/), Mutation Survey (mutationassessor.org) and SIFT (sift.jcvi.org). Protein function data in column three are annotated in the same order (i.e., Mutation Taster/PolyPhen/Mutation Surveyor/SIFT). Protein function symbols are Orange, Yellow, Green. O/O/O/O is most damaging; G/G/G/G is least damaging.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of treating an individual at risk of or suffering from a mitochondrial dysfunction or disorder, autism, and/or Pediatric Acute-onset Neuropsychiatric Syndrome (PANS), the method comprising administering to the individual a therapeutically effective amount of folinic acid, glycine or a pharmaceutically acceptable salt thereof, wherein DNA of the individual encoding one or more proteins selected from the group consisting of aldehyde dehydrogenase 1 family, member L1 (ALDH1L1), aldehyde dehydrogenase 1 family, member L2 (ALDH1L2), folate receptor 1 (FOLR1), folylpolyglutamate synthase (FPGS), glycine cleavage system H protein (GCSH), glycine cleavage system P protein (GLDC), C-1-tetrahydrofolate synthase (cytoplasmic) (MTHFD1), monofunctional C1-tetrahydrofolate synthase, mitochondrial (MTHFD1L), bifunctional methylenetetrahydrofolate dehydrogenase/cyclohydrolase (MTHFD2), methylenetetrahydrofolate dehydrogenase (NADP+ dependent) 2-like (MTHFD2L), 5,10-methenyltetrahydrofolate synthetase (MTHFS), methionine synthase reductase (MTRR), serine hydroxymethyltransferase 1 (SHMT1), serine hydroxymethyltransferase 2 (SHMT2) and solute carrier family 25 (mitochondrial folate carrier) (SLC25A32) includes a loss-of-function mutation.

2. The method of claim 1, wherein, prior to administration, the individual has been determined to possess a loss-of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.

3. The method of claim 1, further comprising determining that the individual possesses a loss-of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32 and administering to the individual a therapeutically effective amount of folinic acid.

4. The method of claim 2, wherein the mitochondrial dysfunction or disorder is selected from the group consisting of functional gastrointestinal disorders, chronic pain disorders, chronic fatigue syndrome, intermittent encephalopathy, dementia and combinations thereof.

5. The method of claim 2, wherein the loss-of-function mutation causes a reduction in levels of folate.

6. (canceled)

7. The method of claim 2, wherein the loss-of-function mutation in ALDH1L1 is or comprises a mutation selected from the group consisting of 23G>D, 117S>L, 333R>Q, 448S>N, 524G>S, 666N>K, 760E>K, 771T>A, 876K>R, frame shift p.Ala107Profs64X, and combinations thereof.

8. The method of claim 2, wherein the loss-of-function mutation in ALDH1L2 is or comprises a mutation selected from the group consisting of 204L>F, 603W>X, 748V>A, 796G>R, 833T>I, 918T>M, and combinations thereof.

9. The method of claim 2, wherein the loss-of-function mutation in FOLR1 is or comprises a mutation consisting of 98R>W.

10. The method of claim 2, wherein the loss-of-function mutation in FPGS is or comprises a mutation selected from the group consisting of 50R>C, 85R>W, 162R>Q, 466R>C, and combinations thereof.

11. The method of claim 2, wherein the loss-of-function mutation in GCSH is or comprises a mutation consisting of 84Y>H.

12. The method of claim 2, wherein the loss-of-function mutation in GLDC is or comprises a mutation selected from the group consisting of 18G>C, 1471>M, 503E>A, 675N>K, 705V>M, 716L>H, 895M>V, 937R>L, 966Q>H, and combinations thereof.

13. The method of claim 2, wherein the loss-of-function mutation in MTHFD1 is or comprises a mutation consisting of 830A>V.

14. The method of claim 2, wherein the loss-of-function mutation in MTHFD1L is or comprises a mutation selected from the group consisting of 31A>G, 520Y>C, 564R>H, 949G>R, and combinations thereof.

15. The method of claim 2, wherein the loss-of-function mutation in MTHFD2 is or comprises a mutation consisting of 263D>G.

16. The method of claim 2, wherein the loss-of-function mutation in MTHFD2L is or comprises a mutation selected from the group consisting of 161G>E, 210V>L, and combinations thereof.

17. The method of claim 2, wherein the loss-of-function mutation in MTHFS is or comprises a mutation selected from the group consisting of 133L>Q, 174E>K, and combinations thereof.

18. The method of claim 2, wherein the loss-of-function mutation in MTRR is or comprises a mutation selected from the group consisting of 3171>T, 517T>A, and combinations thereof.

19. The method of claim 2, wherein the loss-of-function mutation in SHMT1 is or comprises a mutation selected from the group consisting of 1M>R, 1M>K, 191R>C, 344E>Q, and combinations thereof.

20. The method of claim 2, wherein the loss-of-function mutation in SHMT2 is or comprises a mutation selected from the group consisting of 193R>Q, 327R>Q, and combinations thereof.

21. The method of claim 2, wherein the loss-of-function mutation in SLC25A32 is or comprises a mutation selected from the group consisting of 163Y>C, 300Y>C, and combinations thereof.

22.-23. (canceled)

24. The method of claim 2, wherein the method comprises administering to the individual a therapeutically effective amount of folinic acid or a pharmaceutically acceptable salt thereof.

25. The method of claim 2, wherein the method comprises administering to the individual a therapeutically effective amount of glycine or a pharmaceutically acceptable salt thereof.

26. A method of aiding in the selection of a therapy for an individual at risk of or suffering from a mitochondrial dysfunction or disorder, autism, and/or Pediatric Acute-onset Neuropsychiatric Syndrome (PANS), the method comprising:
obtaining a sample of DNA from the individual;
processing the sample to determine whether the individual possesses a loss-of-function mutation in DNA encoding one or more proteins selected from the group consisting of ALDH1L1, ALDH1L2, FOLR1, FPGS, GCSH, GLDC, MTHFD, MTHFD1L, MTHFD2, MTHFD2L, MTHFS, MTRR, SHMT1, SHMT2 and SLC25A32; and
classifying the individual as one that could benefit from therapy with folinic acid, glycine or a pharmaceutically acceptable salt thereof if the step of processing determines that the individual possesses a loss-of-function mutation in DNA encoding one or more proteins selected from the group consisting of ALDH1L1, ALDH1L2, FOLR1, FPGS, GCSH, GLDC, MTHFD, MTHFD1L, MTHFD2, MTHFD2L, MTHFS, MTRR, SHMT1, SHMT2 and SLC25A32.

27.-64. (canceled)

65. A method of classifying an individual at risk of or suffering from a mitochondrial dysfunction or disorder, autism, and/or Pediatric Acute-onset Neuropsychiatric Syndrome (PANS), the method comprising:
obtaining a sample of DNA from the individual;
processing the sample to determine whether the individual possesses a mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32; and
classifying the individual as one that does or does not possess a mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.

66.-105. (canceled)

106. A kit for classifying an individual at risk of or suffering from a mitochondrial dysfunction or disorder, autism, and/or Pediatric Acute-onset Neuropsychiatric Syndrome (PANS), the kit comprising primers for amplifying a target region of DNA that encompasses part or all of the codon for amino acids selected from the group consisting of:
(a) residues 23, 64-107, 117, 333, 448, 524, 666, 760, 771 and 876 of an ALDH1L1 gene product;
(b) residues 204, 603, 748, 796, 833 and 918 of an ALDH1L2 gene product;
(c) residue 98 of a FOLR1 gene product;
(d) residues 50, 85, 162 and 466 of a FPGS gene product;
(e) residue 84 of a GCSH gene product;
(f) residues 18, 147, 503, 675, 705, 716, 895, 937 and 966 of a GLDC gene product;
(g) residue 830 of a MTHFD1 gene product;
(h) residues 31, 520, 564 and 949 of a MTHFD1L gene product;
(i) residue 263 of a MTHFD2 gene product;
(j) residues 161 and 210 of a MTHFD2L gene product;
(k) residues 133 and 174 of a MTHFS gene product;
(l) residues 317 and 517 of a MTRR gene product;
(m) residues 1, 191 and 344 of a SHMT1 gene product;
(n) residues 193 and 327 of a SHMT2 gene product;
(o) residues 163 and 300 of an SLC25A32 gene product; and combinations thereof.

107.-121. (canceled)

122. A kit for classifying an individual at risk of or suffering from a mitochondrial dysfunction or disorder, autism, and/or Pediatric Acute-onset Neuropsychiatric Syndrome (PANS), the kit comprising primers for amplifying a target region of DNA encompassing a region selected from the group consisting of:
(a) residues 23, 64-107, 117, 333, 448, 524, 666, 760, 771 and 876 of an ALDH1L1 gene product;
(b) residues 204, 603, 748, 796, 833 and 918 of an ALDH1L2 gene product;
(c) residue 98 of a FOLR1 gene product;
(d) residues 50, 85, 162 and 466 of a FPGS gene product;
(e) residue 84 of a GCSH gene product;
(f) residues 18, 147, 503, 675, 705, 716, 895, 937 and 966 of a GLDC gene product;
(g) residue 830 of a MTHFD1 gene product;
(h) residues 31, 520, 564 and 949 of a MTHFD1L gene product;
(i) residue 263 of a MTHFD2 gene product;
(j) residues 161 and 210 of a MTHFD2L gene product;
(k) residues 133 and 174 of a MTHFS gene product;
(l) residues 317 and 517 of a MTRR gene product;
(m) residues 1, 191 and 344 of a SHMT1 gene product;
(n) residues 193 and 327 of a SHMT2 gene product;
(o) residues 163 and 300 of an SLC25A32 gene product; and combinations thereof wherein said region includes one or more sites of loss-of-function mutations that are associated with a mitochondrial dysfunction or disorder, autism, and/or Pediatric Acute-onset Neuropsychiatric Syndrome (PANS).

123.-159. (canceled)

160. The method of claim 3, wherein determining that the individual possesses a loss- of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32 comprises requesting sequencing of at least a portion of nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.

161. The method of claim 3, wherein determining that the individual possesses a loss- of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32 comprises sequencing of at least a portion of nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.

162. The method of claim 3, wherein determining that the individual possesses a loss- of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32 comprises requesting genotyping of at least a portion of nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.

163. The method of claim 3, wherein determining that the individual possesses a loss- of-function mutation in DNA encoding one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32 comprises genotyping of at least a portion of nuclear DNA that encodes one or more proteins selected from the group consisting of ALDH1L1, ALDH1L2, FOLR1, FPGS, GCSH, GLDC, MTHFD1, MTHFD1L, MTHFD2, MTHFD2L, MTHFS, MTRR, SHMT1, SHMT2 and SLC25A32.
